(19) United States
(12) Patent Application Publication
(10) Pub. No.: US 2025/0263494 A1
(43) Pub. Date: Aug. 21, 2025

(54) ANTI-TL1A ANTIBODY FORMULATIONS

(71) Applicant: Cephalon LLC, West Chester, PA (US)

(72) Inventor: Shyam Bhaskerbhai Mehta, Malvern, PA (US)

(73) Assignee: Cephalon LLC, West Chester, PA (US)

(21) Appl. No.: 19/035,583

(22) Filed: Jan. 23, 2025

Related U.S. Application Data
(63) Continuation of application No. PCT/US23/71088, filed on Jul. 27, 2023.
(60) Provisional application No. 63/369,638, filed on Jul. 27, 2022.

(51) Int. Cl. C07K 16/28; A61K 47/18; A61K 47/22; A61K 47/26

(52) U.S. Cl. CPC C07K 16/2875; A61K 47/183; A61K 47/22; A61K 47/26; C07K 2317/14; C07K 2317/52; C07K 2317/565; C07K 2317/94

(57) ABSTRACT

Pharmaceutical formulations comprising an antibody or antigen-binding fragment thereof that specifically binds to TL1A are provided. The formulations have advantageous properties including, for example, stability.

### CROSS-REFERENCE TO RELATED APPLICATION

This application is a continuation of International Application No. PCT/US2023/071088, filed Jul. 27, 2023, which claims the benefit of priority to U.S. Provisional Application No. 63/369,638, filed Jul. 27, 2022, the entire contents of which are incorporated herein by reference for all purposes.

### REFERENCE TO SEQUENCE LISTING SUBMITTED ELECTRONICALLY

This patent application is filed with a sequence listing in electronic format. The Sequence Listing is provided as a file entitled “2025-01-23_01183-0324-00US_SequenceListing_St26.xml,” which was created on Jul. 20, 2023, and which is 67,054 bytes in size. The information in the electronic format of the sequence listing is incorporated herein by reference in its entirety.

### BACKGROUND

TNF-like ligand 1A (TL1A, syn. TNF superfamily member 15 (TNFSF15); TL1 and VEGI) is a member of the tumor necrosis factor superfamily, which is expressed by antigen presenting cells (including dendritic cells, B cells and macrophages), CD4+ and CD8+ T cells and endothelial cells. TL1A can be expressed on the cell surface or secreted as a soluble cytokine. The receptor for TL1A, Death Receptor 3 (DR3) is expressed by a variety of cells, including CD4+ and CD8+ T cells, NK cells, NKT cells and FOXP3+ regulatory T (Treg) cells and type-2 and type-3 innate lymphoid cells (ILC2 and ILC3).

TL1A can also bind a decoy receptor (DcR3), which is a competitive inhibitor of DR3. DcR3 also acts as a decoy receptor for Fas-ligand (Fas-L) and lymphotoxin-like inducible protein that competes with glycoprotein D for binding herpesvirus entry mediator on T-cells (LIGHT). Accordingly, DcR3 is an important regulator of several signal transduction pathways.

The TL1A/DR3 signalling pathway has been implicated in several biological systems, which are associated with human diseases. For example, TL1A has been shown to play a role in immunity, angiogenesis, and homeostasis of barrier tissues. Inhibiting TL1A interaction with DR3 also has been shown to promote a therapeutic benefit in several immune-mediated conditions, such as experimental autoimmune encephalomyelitis (EAE; a model of multiple sclerosis), colitis, ulcerative colitis, Crohn's disease, inflammatory bowel disease, skin disease, asthma and arthritis. Thus, antibodies that bind to TL1A have been proposed as treatments for these disease.

However, in order to use such antibodies and provide treatment for these diseases, stable formulations for the antibodies are needed.

### BRIEF SUMMARY

Provided herein is a pharmaceutical formulation, comprising: (a) about 100 mg/mL to about 250 mg/mL of an antibody or antigen-binding fragment thereof that specifically binds to TNF-like ligand 1A (TL1A); wherein the antibody or antigen-binding fragment thereof comprises: a heavy chain variable region CDR1 comprising the amino acid sequence of SEQ ID NO: 1, a heavy chain variable region CDR2 comprising the amino acid sequence of SEQ ID NO: 2, a heavy chain variable region CDR3 comprising the amino acid sequence of SEQ ID NO: 3, a light chain variable region CDR1 comprising the amino acid sequence of SEQ ID NO: 4, a light chain variable region CDR2 comprising the amino acid sequence of SEQ ID NO: 5, and a light chain variable region CDR3 comprising the amino acid sequence of SEQ ID NO: 6; (b) about 5 mM to about 15 mM Histidine; (c) about 50 mM to about 150 mM Arginine-Hydrochloride (Arg-HCl); (d) about 2.5% (w/v) to about 7.5% (w/v) Sucrose; and (e) about 0.01% (w/v) to about 0.03% (w/v) Polysorbate-80. In some aspects, the antibody or antigen-binding fragment thereof comprises a heavy chain variable region comprising the amino acid sequence of SEQ ID NO: 7 and a light chain variable region comprising the amino acid sequence of SEQ ID NO: 8. In some aspects, the antibody or antigen-binding fragment comprises an IgG1 constant region. In some aspects, the antibody or antigen-binding fragment thereof comprises a heavy chain comprising the amino acid sequence of SEQ ID NO: 9 and a light chain comprising the amino acid sequence of SEQ ID NO: 10.

In some aspects, the pharmaceutical formulation disclosed herein comprises about 100, about 150, about 200, about 225, or about 250 mg/mL of the antibody or antigen-binding fragment thereof. In some aspects, the pharmaceutical formulation disclosed herein comprises about 5 mM, about 10 mM, or about 15 mM Histidine. In some aspects, the pharmaceutical formulation disclosed herein comprises about 50 mM, about 100 mM, or about 150 mM Arginine-Hydrochloride (Arg-HCl). In some aspects, the pharmaceutical formulation disclosed herein comprises about 2.5%, about 5%, or about 7.5% (w/v) Sucrose. In some aspects, the pharmaceutical formulation disclosed herein comprises about 0.01%, about 0.02%, or about 0.03% (w/v) Polysorbate-80.

In some aspects, the pharmaceutical formulation disclosed herein comprises about 250 mg/mL of the antibody or antigen binding fragment thereof, about 10 mM Histidine, about 100 mM Arginine-Hydrochloride (Arg-HCl), about 5% (w/v) Sucrose, and about 0.02% (w/v) Polysorbate-80.

In some aspects, the pharmaceutical formulation disclosed herein comprises about 200 mg/mL of the antibody or antigen binding fragment thereof, about 10 mM Histidine, about 100 mM Arginine-Hydrochloride (Arg-HCl), about 5% (w/v) Sucrose, and about 0.02% (w/v) Polysorbate-80.

In some aspects, the pharmaceutical formulation disclosed herein comprises about 150 mg/mL of the antibody or antigen binding fragment thereof, about 10 mM Histidine, about 100 mM Arginine-Hydrochloride (Arg-HCl), about 5% (w/v) Sucrose, and about 0.02% (w/v) Polysorbate-80.

In some aspects, the pharmaceutical formulation is lyophilized. In some aspects, the pharmaceutical formulation is liquid. In some aspects, the pharmaceutical formulation has a pH of 6.0±0.5 after storage at room temperature for 24 hours or at 2-8° C. for 24 hours, 72 hours, or 10 days.

In some aspects, the pharmaceutical formulation has an osmolality of from 200 mOsm/kg to 500 mOsm/kg after storage at room temperature for 24 hours or at 2-8° C. for 24 hours, 72 hours, or 10 days. In some aspects, the pharmaceutical formulation has at least 99% antibody monomer content after storage at 2-8° C. for 24 hours, 72 hours, or 10 days. In some aspects, the pharmaceutical formulation has no significant change in charge heterogeneity profile after storage at 2-8° C. for 24 hours, 72 hours, or 10 days. In some aspects, the pharmaceutical formulation has no significant change in purity after storage at room temperature for 24 hours or at 2-8° C. for 24 hours, 72 hours, or 10 days. In some aspects, the pharmaceutical formulation has at least 90% purity after storage at room temperature for 24 hours or at 2-8° C. for 24 hours, 72 hours, or 10 days. In some aspects, the pharmaceutical formulation has no significant change in particle concentration after storage at room temperature for 24 hours or at 2-8° C. for 24 hours, 72 hours, or 10 days. In some aspects, the pharmaceutical formulation has no significant difference in visual appearance after storage at 2-8° C. for up to 36 months. In some aspects, the pharmaceutical formulation disclosed herein has no significant difference in protein concentration, osmolality or viscosity after storage at 2-8° C., 25° C. or 40° C. for up to 36 months. In some aspects, the pharmaceutical formulation disclosed herein has ≥(more than or equal to) 95% monomer content, ≤(less than or equal to) 5.0% dimer content, or no significant difference in low molecular weight species content after storage at 2-8° C. for up to 36 months. In some aspects, the pharmaceutical formulation disclosed herein has ≥(more than or equal to) 90% purity after storage at 2-8° C. for up to 36 months. In some aspects, the pharmaceutical formulation disclosed herein has from 50% to 90% main species content, from 10% to 40% acidic species content, and/or from 0% to 20% basic species content after storage at 2-8° C. for up to 36 months. In some aspects, the pharmaceutical formulation disclosed herein has no significant difference in sub-visible particle content after storage at 2-8° C., 25° C., or 40° C. for up to 36 months. In some aspects, the pharmaceutical formulation disclosed herein has no significant difference in oxidation of methionine 81 and/or methionine 254 of TEV-48574, and/or deamidation of asparagine 317 of TEV-48574 after storage at 2-8° C., 25° C., or 40° C. for up to 36 months. In some aspects, the pharmaceutical formulation disclosed herein has from 70% to 135% relative potency measured by enzyme-linked immunosorbent assay (ELISA) after storage at 2-8° C. for up to 36 months. In some aspects, the pharmaceutical formulation disclosed herein has no significant difference in thermal stability after storage at 2-8° C., 25° C., or 40° C. for up to 6 months. In some aspects, the pharmaceutical formulation disclosed herein has no significant difference in thermal stability after storage at 2-8° C. for up to 36 months. In some aspects, the pharmaceutical formulation disclosed herein has no significant difference in secondary and/or tertiary protein structure after storage at 2-8° C., 25° C., or 40° C. for up to 3 months. In some aspects, the pharmaceutical formulation disclosed herein has no significant difference in secondary protein structure after storage at 2-8° C. for up to 36 months. In some aspects, the pharmaceutical formulation disclosed herein has no significant difference in concentration of Polysorbate-80 after storage at 2-8° C. for up to 24 months.

Also provided herein is a container comprising the pharmaceutical formulation disclosed herein. In some aspects, the container is a glass vial. In some aspects, the container is a glass vial having a fill volume of 3 mL. In some aspects, the container is a glass vial having a fill volume of 3 mL or less. In some aspects, the container is a syringe, e.g., pre-filled syringe. In some aspects, the pre-filled syringe is in a volume of 2 mL or less.

In some aspects, the antibody or antigen-binding fragment thereof is present in the pharmaceutical formulation or container disclosed herein. In some aspects, the antibody or antigen-binding fragment thereof is formulated in a volume of 3 mL or less. In some aspects, the antibody or antigen-binding fragment thereof is formulated in a volume of 2 mL or less.

In some aspects, provided herein is a method of treating a disease in a subject in need thereof, the method comprising administering to the subject any of the pharmaceutical formulations provided herein, including a pharmaceutical formulation in any of the containers provided herein. In some aspects, the disease is a respiratory tract disease, a gastrointestinal disease, a skin disease, or an arthritis.

In some aspects, the respiratory tract disease is an asthma, a chronic obstructive pulmonary disease (COPD), a pulmonary fibrosis, a pulmonary sarcoidosis, an allergic rhinitis, or a cystic fibrosis.

In some aspects, the gastrointestinal is an inflammatory bowel disease, a Crohn's disease, a colitis, an ulcerative colitis, an eosinophilic esophagitis, or an irritable bowel syndrome.

In some aspects, the arthritis is a rheumatoid arthritis.

In some aspects, the skin disease is an atopic dermatitis, an eczema, or a scleroderma.

In some aspects, the pharmaceutical formulation is administered intravenously. In some aspects, the pharmaceutical formulation is administered subcutaneously.

Also provided herein is a composition for use in accordance with the methods disclosed herein.

Provided herein is a pharmaceutical formulation, comprising: (a) 150 mg/mL of an antibody or antigen-binding fragment thereof that specifically binds to TNF-like ligand 1A (TL1A); wherein the antibody or antigen-binding fragment thereof comprises: (i) a heavy chain variable region comprising the amino acid sequence of SEQ ID NO: 7 and a light chain variable region comprising the amino acid sequence of SEQ ID NO: 8; (ii) a heavy chain variable region CDR1 comprising the amino acid sequence of SEQ ID NO: 1, a heavy chain variable region CDR2 comprising the amino acid sequence of SEQ ID NO: 2, a heavy chain variable region CDR3 comprising the amino acid sequence of SEQ ID NO: 3, a light chain variable region CDR1 comprising the amino acid sequence of SEQ ID NO: 4, a light chain variable region CDR2 comprising the amino acid sequence of SEQ ID NO: 5, and a light chain variable region CDR3 comprising the amino acid sequence of SEQ ID NO: 6; or (iii) a heavy chain comprising the amino acid sequence of SEQ ID NO: 9 and a light chain comprising the amino acid sequence of SEQ ID NO: 10; (b) 10 mM Histidine; (c) 100 mM Arginine-Hydrochloride (Arg-HCl); (d) 5% (w/v) Sucrose; and (e) 0.02% (w/v) Polysorbate-80. In some aspects, the antibody or antigen-binding fragment of (i) or (ii) comprises an IgG1 constant region.

In some aspects, the pharmaceutical formulation is lyophilized. In some aspects, the pharmaceutical formulation is liquid. In some aspects, the pharmaceutical formulation has a pH of 6.0±0.5 after storage at room temperature for 24 hours or at 2-8° C. for 24 hours, 72 hours, or 10 days.

In some aspects, the pharmaceutical formulation has an osmolality of from 200 mOsm/kg to 500 mOsm/kg after storage at room temperature for 24 hours or at 2-8° C. for 24 hours, 72 hours, or 10 days. In some aspects, the pharmaceutical formulation has at least 99% antibody monomer content after storage at 2-8° C. for 24 hours, 72 hours, or 10 days. In some aspects, the pharmaceutical formulation has no significant change in charge heterogeneity profile after storage at 2-8° C. for 24 hours, 72 hours, or 10 days. In some aspects, the pharmaceutical formulation has no significant change in purity after storage at room temperature for 24 hours or at 2-8° C. for 24 hours, 72 hours, or 10 days. In some aspects, the pharmaceutical formulation has at least 90% purity after storage at room temperature for 24 hours or at 2-8° C. for 24 hours, 72 hours, or 10 days. In some aspects, the pharmaceutical formulation has no significant change in particle concentration after storage at room temperature for 24 hours or at 2-8° C. for 24 hours, 72 hours, or 10 days. In some aspects, the pharmaceutical formulation has no significant difference in visual appearance after storage at 2-8° C. for up to 36 months. In some aspects, the pharmaceutical formulation disclosed herein has no significant difference in protein concentration, osmolality or viscosity after storage at 2-8° C., 25° C. or 40° C. for up to 36 months. In some aspects, the pharmaceutical formulation disclosed herein has ≥(more than or equal to) 95% monomer content, ≤(less than or equal to) 5.0% dimer content, or no significant difference in low molecular weight species content after storage at 2-8° C. for up to 36 months. In some aspects, the pharmaceutical formulation disclosed herein has ≥(more than or equal to) 90% purity after storage at 2-8° C. for up to 36 months. In some aspects, the pharmaceutical formulation disclosed herein has from 50% to 90% main species content, from 10% to 40% acidic species content, and/or from 0% to 20% basic species content after storage at 2-8° C. for up to 36 months. In some aspects, the pharmaceutical formulation disclosed herein has no significant difference in sub-visible particle content after storage at 2-8° C., 25° C., or 40° C. for up to 36 months. In some aspects, the pharmaceutical formulation disclosed herein has no significant difference in oxidation of methionine 81 and/or methionine 254 of TEV-48574, and/or deamidation of asparagine 317 of TEV-48574 after storage at 2-8° C., 25° C., or 40° C. for up to 36 months. In some aspects, the pharmaceutical formulation disclosed herein has from 70% to 135% relative potency measured by enzyme-linked immunosorbent assay (ELISA) after storage at 2-8° C. for up to 36 months. In some aspects, the pharmaceutical formulation disclosed herein has no significant difference in thermal stability after storage at 2-8° C., 25° C., or 40° C. for up to 6 months. In some aspects, the pharmaceutical formulation disclosed herein has no significant difference in thermal stability after storage at 2-8° C. for up to 36 months. In some aspects, the pharmaceutical formulation disclosed herein has no significant difference in secondary and/or tertiary protein structure after storage at 2-8° C., 25° C., or 40° C. for up to 3 months. In some aspects, the pharmaceutical formulation disclosed herein has no significant difference in secondary protein structure after storage at 2-8° C. for up to 36 months. In some aspects, the pharmaceutical formulation disclosed herein has no significant difference in concentration of Polysorbate-80 after storage at 2-8° C. for up to 24 months.

Also provided herein is a container comprising the pharmaceutical formulation disclosed herein. In some aspects, the container is a glass vial. In some aspects, the container is a glass vial having a fill volume of 3 mL.

In some aspects, the antibody or antigen-binding fragment thereof is present in the pharmaceutical formulation or container disclosed herein.

In some aspects, provided herein is a method of treating a disease in a subject in need thereof, the method comprising administering to the subject any of the pharmaceutical formulations provided herein, including a pharmaceutical formulation in any of the containers provided herein. In some aspects, the disease is a respiratory tract disease, a gastrointestinal disease, a skin disease, or an arthritis.

In some aspects, the respiratory tract disease is an asthma, a chronic obstructive pulmonary disease (COPD), a pulmonary fibrosis, a pulmonary sarcoidosis, an allergic rhinitis, or a cystic fibrosis.

In some aspects, the gastrointestinal is an inflammatory bowel disease, a Crohn's disease, a colitis, an ulcerative colitis, an eosinophilic esophagitis, or an irritable bowel syndrome.

In some aspects, the arthritis is a rheumatoid arthritis.

In some aspects, the skin disease is an atopic dermatitis, an eczema, or a scleroderma.

Also provided herein is a composition for use in accordance with the methods disclosed herein.

### BRIEF DESCRIPTION OF THE FIGURES

FIGS. 1A-1C show the percent (%) monomer composition of three TEV-48574 formulations (F1-F3) after storage at 2-8° C. (FIG. 1A), 25° C. (FIG. 1), and 40° C. (FIG. 1C).

FIGS. 2A-2C show the percent (%) dimer composition of three TEV-48574 formulations (F1-F3) after storage at 2-8° C. (FIG. 2A), 25° C. (FIG. 2B), and 40° C. (FIG. 2C).

FIGS. 3A-3C shows the percent (%) low molecular weight (LWM) protein fragment composition of three TEV-48574 formulations (F1-F3) after storage at 2-8° C. (FIG. 3A), 25° C. (FIG. 3B), and 40° C. (FIG. 3C).

FIGS. 4A-4C show the percent (%) purity of three TEV-48574 formulations (F1-F3) after storage at 2-8° C. (FIG. 4A), 25° C. (FIG. 4B), and 40° C. (FIG. 4C), determined using CGE-Non-reducing conditions (% IgG+125 kDa peak).

FIGS. 5A-5C show the percent (%) purity of three TEV-48574 formulations (F1-F3) after storage at 2-8° C. (FIG. 5A), 25° C. (FIG. 5B), and 40° C. (FIG. 5C), determined using CGE-Reducing conditions (% Heavy chain+light chain).

FIGS. 6A-6C show the charge heterogeneity profile for Formulations 1-3 (F1-F3) after storage at 2-8° C. (FIG. 6A), 25° C. (FIG. 6B), and 40° C. (FIG. 6C), as percent (%) main species (main peak).

FIGS. 7A-7C show the charge heterogeneity profile for Formulations 1-3 (F1-F3) after storage at 2-8° C. (FIG. 7A), 25° C. (FIG. 7B), and 40° C. (FIG. 7C), represented as percent (%) acidic species (acidic peak).

FIGS. 8A-8C show the charge heterogeneity profile for Formulations 1-3 (F1-F3) after storage at 2-8° C. (FIG. 8A), 25° C. (FIG. 8B) and 40° C. (FIG. 8C), represented as percent (%) basic species (basic peak).

FIG. 9A shows the thermal stability of Formulations 1-3 (F1-F3) at time zero (T0) and after storage at 2-8° C., 25° C. and 40° C. for 3 months.

FIG. 9B shows the thermal stability of Formulations 1-3 (F1-F3) at time zero (T0) and after storage at 2-8° C., 25° C. and 40° C. for 6 months.

FIG. 9C shows the thermal stability of Formulations 1-3 (F1-F3) at time zero (T0) and after storage at 2-8° C. for 36 months.

FIG. 10A shows the secondary structure of Formulations 1-3 at time zero (T0) and after 3 months of storage at 2-8° C., 25° C., and 40° C. using far ultraviolet (UV) circular dichroism (CD).

FIG. 10B (shows the secondary structure of Formulations 1-3 at time zero (T0) and after 24 months of storage at 2-8° C. using far UV CD.

FIG. 10C shows the secondary structure of Formulations 1-3 at time zero (T0) and after 36 months of storage at 2-8° C. using far UV CD.

FIG. 10D shows the tertiary structure of Formulations 1-3 at time zero (T0) and after 6 months of storage at 2-8° C., 25° C., and 40° C. using near UV CD.

FIG. 10E shows the tertiary structure of Formulations 1-3 at time zero (T0) and after 24 months of storage at 2-8° C. using near UV CD.

FIG. 10F shows the tertiary structure of Formulations 1-3 at time zero (T0) and after 36 months of storage at 2-8° C. using near UV CD.

FIG. 11A shows the secondary structure of Formulation 3 stored in Nipro prefilled syringes (PFS) at time zero (T0) and after 3 months (3M), 6 months (6M), and 24 months (24M) of storage at 2-8° C., and after 3 months (3M) and 6 months (6M) of storage at 25° C. and 40° C. using far UV CD.

FIG. 11B shows the tertiaty structure of Formulation 3 stored in Nipro prefilled syringes (PFS) at time zero (T0) and after 3 months (3M), 6 months (6M), and 24 months (24M) of storage at 2-8° C., and after 3 months (3M) and 6 months (6M) of storage at 25° C. and 40° C. using near UV CD.

FIG. 12A shows the percent (%) monomer composition of a lyophilized TEV-48574 formulation at 100 mg/mL (F4) and a liquid TEV-48574 formulation at 200 mg/mL (F5) after storage at 2-8° C. for up to 24 months.

FIG. 12B shows the percent (%) dimer composition of a lyophilized TEV-48574 formulation at 100 mg/mL (F4) and a liquid TEV-48574 formulation at 200 mg/mL (F5) after storage at 2-8° C. for up to 24 months.

FIG. 12C shows the percent (%) low molecular weight species of a lyophilized TEV-48574 formulation at 100 mg/mL (F4) and a liquid TEV-48574 formulation at 200 mg/mL (F5) after storage at 2-8° C. for up to 24 months.

FIG. 13A shows the percent (%) purity of two TEV-48574 formulations (F4 and F5) after storage at 2-8° C. for up to 24 months, determined using CGE-Non-reducing conditions (% IgG+125 kDa peak).

FIG. 13B shows the percent (%) purity of two TEV-48574 formulations (F4 and F5) after storage at 2-8° C. for up to 24 months, determined using CGE-Reducing conditions (% Heavy chain (HC)+light chain (LC)).

FIG. 14A shows the charge heterogeneity profile for Formulations 4 and 5 (F4 and F5) after storage at 2-8° C. for up to 24 months as percent (%) main species (main peak).

FIG. 14B shows the charge heterogeneity profile for Formulations 4 and 5 (F4 and F5) after storage at 2-8° C. for up to 24 months, represented as percent (%) acidic species (acidic peak).

FIG. 14C shows the charge heterogeneity profile for Formulations 4 and 5 (F4 and F5) after storage at 2-8° C. for up to 24 months, represented as percent (%) basic species (basic peak).

FIG. 15A shows the secondary structure of Formulation 4 at time zero (T0) and after 3 months (3M), 6 months (6M) and 12 months (12M) of storage at 25° C., and of Formulation 5 at T0, and after 3M and 12M of storage at 25° C. using far UV CD.

FIG. 15B shows the tertiary structure of Formulation 4 at time zero (T0) and after 3 months (3M) and 12 months (12M) of storage at 25° C., and of Formulation 5 at T0, 3M, and after 6 months (6M) and 12M of storage at 25° C. using near UV CD.

FIG. 16 shows the viscosity at 20° C. for liquid formulations with increasing concentrations of TEV-48574 (0 to 150 mg/mL), with either 100 mM arginine-HCl added as an excipient (1A) or without arginine-HCl (1B) added.

FIG. 17A shows the percent (%) monomer composition of two liquid TEV-48574 formulations at 150 mg/mL (1A) and 100 mg/mL (2A) with 100 mM arginine-HCl added, and two liquid TEV-48574 formulations at 150 mg/mL (1B) and 100 mg/mL (2B) without arginine-HCl after storage at 40° C. for up to 8 weeks.

FIG. 17B shows the percent (%) dimer composition of two liquid TEV-48574 formulations at 150 mg/mL (1A) and 100 mg/mL (2A) with 100 mM arginine-HCl added, and two liquid TEV-48574 formulations at 150 mg/mL (1B) and 100 mg/mL (2B) without arginine-HCl after storage at 40° C. for up to 8 weeks.

FIG. 17C shows the percent (%) fragments of two liquid TEV-48574 formulations at 150 mg/mL (1A) and 100 mg/mL (2A) with 100 mM arginine-HCl added, and two liquid TEV-48574 formulations at 150 mg/mL (1B) and 100 mg/mL (2B) without arginine-HCl after storage at 40° C. for up to 8 weeks.

FIG. 18A shows the percent (%) purity of two liquid TEV-48574 formulations at 150 mg/mL (1A) and 100 mg/mL (2A) with 100 mM arginine-HCl added, and two liquid TEV-48574 formulations at 150 mg/mL (1B) and 100 mg/mL (2B) without arginine-HCl after storage at 40° C. for up to 8 weeks, determined using CGE-Non-reducing conditions (% IgG+125 kDa peak).

FIG. 18B shows the percent (%) purity of two liquid TEV-48574 formulations at 150 mg/mL (1A) and 100 mg/mL (2A) with 100 mM arginine-HCl added, and two liquid TEV-48574 formulations at 150 mg/mL (1B) and 100 mg/mL (2B) without arginine-HCl after storage at 40° C. for up to 8 weeks, determined using CGE-Reducing conditions (% Heavy chain (HC)+light chain (LC)).

FIG. 19A shows the charge heterogeneity profile of two liquid TEV-48574 formulations at 150 mg/mL (1A) and 100 mg/mL (2A) with 100 mM arginine-HCl added, and two liquid TEV-48574 formulations at 150 mg/mL (1B) and 100 mg/mL (2B) without arginine-HCl after storage at 40° C. for up to 8 weeks as percent (%) main species (main peak).

FIG. 19B shows the charge heterogeneity profile of two liquid TEV-48574 formulations at 150 mg/mL (1A) and 100 mg/mL (2A) with 100 mM arginine-HCl added, and two liquid TEV-48574 formulations at 150 mg/mL (1B) and 100 mg/mL (2B) without arginine-HCl after storage at 40° C. for up to 8 weeks, represented as percent (%) acidic species (acidic peak).

FIG. 19C shows the charge heterogeneity profile of two liquid TEV-48574 formulations at 150 mg/mL (1A) and 100 mg/mL (2A) with 100 mM arginine-HCl added, and two liquid TEV-48574 formulations at 150 mg/mL (1B) and 100 mg/mL (2B) without arginine-HCl after storage at 40° C. for up to 8 weeks, represented as percent (%) basic species (basic peak).

FIG. 20 shows the thermal stability of two liquid TEV-48574 formulations at 150 mg/mL (1A) and 100 mg/mL (2A) with 100 mM arginine-HCl added, and two liquid TEV-48574 formulations at 150 mg/mL (1B) and 100 mg/mL (2B) without arginine-HCl at time zero (T0).

### DETAILED DESCRIPTION

In order that the present disclosure can be more readily understood, certain terms are first defined. As used in this application, except as otherwise expressly provided herein, each of the following terms shall have the meaning set forth below. Additional definitions are set forth throughout the application.

### Definitions

Various terms relating to aspects of the disclosure are used throughout the specification and claims. Such terms are to be given their ordinary meaning in the art, unless otherwise indicated. Other specifically defined terms are to be construed in a manner consistent with the definition provided herein.

The term “antibody” means an immunoglobulin molecule that recognizes and specifically binds to a target, such as a protein, polypeptide, peptide, carbohydrate, polynucleotide, lipid, or combinations of the foregoing through at least one antigen recognition site within the variable region of the immunoglobulin molecule. As used herein, the term “antibody” encompasses intact polyclonal antibodies, intact monoclonal antibodies, chimeric antibodies, humanized antibodies, human antibodies, fusion proteins comprising an antibody, and any other modified immunoglobulin molecule so long as the antibodies exhibit the desired biological activity. An antibody can be of any the five major classes of immunoglobulins: IgA, IgD, IgE, IgG, and IgM, or subclasses (isotypes) thereof (e.g. IgG1, IgG2, IgG3, IgG4, IgA1 and IgA2), based on the identity of their heavy-chain constant domains referred to as alpha, delta, epsilon, gamma, and mu, respectively. The different classes of immunoglobulins have different and well known subunit structures and three-dimensional configurations. Antibodies can be naked or conjugated to other molecules such as radioisotopes.

Where not expressly stated, and unless the context indicates otherwise, the term “antibody” includes monospecific, bispecific, or multi-specific antibodies. In some aspects, the antibody is a bispecific antibody. The term “bispecific antibodies” refers to antibodies that bind to two different epitopes.

The term “antibody fragment” refers to a portion of an intact antibody. An “antigen-binding fragment,” “antigen-binding domain,” or “antigen-binding region,” refers to a portion of an intact antibody that binds to an antigen. In the context of a bispecific antibody, an “antigen-binding fragment” binds two antigens. An antigen-binding fragment can contain an antigen recognition site of an intact antibody (e.g., complementarity determining regions (CDRs) sufficient to specifically bind antigen). Examples of antigen-binding fragments of antibodies include, but are not limited to Fab, Fab′, F(ab′)2, and Fv fragments, linear antibodies, and single chain antibodies. An antigen-binding fragment of an antibody can be derived from any animal species, such as rodents (e.g., mouse, rat, or hamster) and humans or can be artificially produced.

A “monoclonal” antibody or antigen-binding fragment thereof refers to a homogeneous antibody or antigen-binding fragment population involved in the highly specific binding of a single antigenic determinant, or epitope. This is in contrast to polyclonal antibodies that typically include different antibodies directed against different antigenic determinants. The term “monoclonal” antibody or antigen-binding fragment thereof encompasses both intact and full-length monoclonal antibodies as well as antibody fragments (such as Fab, Fab′, F(ab′)2, Fv), single chain (scFv) mutants, fusion proteins comprising an antibody portion, and any other modified immunoglobulin molecule comprising an antigen recognition site. Furthermore, “monoclonal” antibody or antigen-binding fragment thereof refers to such antibodies and antigen-binding fragments thereof made in any number of manners including but not limited to by hybridoma, phage selection, recombinant expression, and transgenic animals.

As used herein, the terms “variable region” or “variable domain” are used interchangeably and are common in the art. The variable region typically refers to a portion of an antibody, generally, a portion of a light or heavy chain, typically about the amino-terminal 110 to 120 amino acids or 110 to 125 amino acids in the mature heavy chain and about 90 to 115 amino acids in the mature light chain, which differ in sequence among antibodies and are used in the binding and specificity of a particular antibody for its particular antigen. The variability in sequence is concentrated in those regions called complementarity determining regions (CDRs) while the more highly conserved regions in the variable domain are called framework regions (FR). Without wishing to be bound by any particular mechanism or theory, it is believed that CDRs of the light and heavy chains are primarily responsible for the interaction and specificity of the antibody with antigen. In some aspects of the present disclosure, the variable region is a human variable region. In some aspects of the present disclosure, the variable region comprises rodent or murine CDRs and human framework regions (FRs). In particular aspects of the present disclosure, the variable region is a primate (e.g., non-human primate) variable region. In some aspects of the present disclosure, the variable region comprises rodent or murine CDRs and primate (e.g., non-human primate) framework regions (FRs).

The terms “VL” and “VL domain” are used interchangeably to refer to the light chain variable region of an antibody.

The terms “VH” and “VH domain” are used interchangeably to refer to the heavy chain variable region of an antibody.

The term “Kabat numbering” and like terms are recognized in the art and refer to a system of numbering amino acid residues in the heavy and light chain variable regions of an antibody or an antigen-binding fragment thereof. In some aspects, CDRs can be determined according to the Kabat numbering system (see, e.g., Kabat EA & Wu TT (1971) Ann NY Acad Sci 190: 382-391 and Kabat E A et al., (1991) Sequences of Proteins of Immunological Interest, Fifth Edition, U.S. Department of Health and Human Services, NIH Publication No. 91-3242). Using the Kabat numbering system, CDRs within an antibody heavy chain molecule are typically present at amino acid positions 31 to 35, which optionally can include one or two additional amino acids, following 35 (referred to in the Kabat numbering scheme as 35A and 35B) (CDR1), amino acid positions 50 to 65 (CDR2), and amino acid positions 95 to 102 (CDR3). Using the Kabat numbering system, CDRs within an antibody light chain molecule are typically present at amino acid positions 24 to 34 (CDR1), amino acid positions 50 to 56 (CDR2), and amino acid positions 89 to 97 (CDR3).

Chothia refers instead to the location of the structural loops (Chothia and Lesk, J. Mol. Biol. 196:901-917 (1987)). The end of the Chothia CDR-H1 loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34). The AbM hypervariable regions represent a compromise between the Kabat CDRs and Chothia structural loops, and are used by Oxford Molecular's AbM antibody modeling software.

| Loop | Kabat | AbM | Chothia |
|---|---|---|---|
| L1 | L24-L34 | L24-L34 | L24-L34 |
| L2 | L50-L56 | L50-L56 | L50-L56 |
| L3 | L89-L97 | L89-L97 | L89-L97 |
| H1 | H31-H35B | H26-H35B | H26-H32 . . . 34 |

(Kabat Numbering)

| | | | |
|---|---|---|---|
| H1 | H31-H35 | H26-H35 | H26-H32 |

(Chothia Numbering)

| | | | |
|---|---|---|---|
| H2 | H50-H65 | H50-H58 | H52-H56 |
| H3 | H95-H102 | H95-H102 | H95-H102 |

As used herein, the term “constant region” or “constant domain” are interchangeable and have the meaning common in the art. The constant region is an antibody portion, e.g., a carboxyl terminal portion of a light and/or heavy chain which is not directly involved in binding of an antibody to antigen but which can exhibit various effector functions, such as interaction with the Fc receptor. The constant region of an immunoglobulin molecule generally has a more conserved amino acid sequence relative to an immunoglobulin variable domain. In some aspects, an antibody or antigen-binding fragment comprises a constant region or portion thereof that is sufficient for antibody-dependent cell-mediated cytotoxicity (ADCC).

As used herein, the term “heavy chain” when used in reference to an antibody can refer to any distinct type, e.g., alpha (α), delta (δ), epsilon (ε), gamma (γ), and mu (μ), based on the amino acid sequence of the constant domain, which give rise to IgA, IgD, IgE, IgG, and IgM classes of antibodies, respectively, including subclasses of IgG, e.g., IgG1, IgG2, IgG3, and IgG4. Heavy chain amino acid sequences are well known in the art. In some aspects of the present disclosure, the heavy chain is a human heavy chain.

As used herein, the term “light chain” when used in reference to an antibody can refer to any distinct type, e.g., kappa (κ) or lambda (λ) based on the amino acid sequence of the constant domains. Light chain amino acid sequences are well known in the art. In some aspects of the present disclosure, the light chain is a human light chain.

The term “chimeric” antibodies or antigen-binding fragments thereof refers to antibodies or antigen-binding fragments thereof wherein the amino acid sequence is derived from two or more species. Typically, the variable region of both light and heavy chains corresponds to the variable region of antibodies or antigen-binding fragments thereof derived from one species of mammals (e.g. mouse, rat, rabbit, etc.) with the desired specificity, affinity, and capability while the constant regions are homologous to the sequences in antibodies or antigen-binding fragments thereof derived from another (usually human) to avoid eliciting an immune response in that species.

The term “humanized” antibody or antigen-binding fragment thereof refers to forms of non-human (e.g. murine) antibodies or antigen-binding fragments that are specific immunoglobulin chains, chimeric immunoglobulins, or fragments thereof that contain minimal non-human (e.g., murine) sequences. Typically, humanized antibodies or antigen-binding fragments thereof are human immunoglobulins in which residues from the complementary determining region (CDR) are replaced by residues from the CDR of a non-human species (e.g. mouse, rat, rabbit, hamster) that have the desired specificity, affinity, and capability (“CDR grafted”) (Jones et al., Nature 321:522-525 (1986); Riechmann et al., Nature 332:323-327 (1988); Verhoeyen et al., Science 239:1534-1536 (1988)). In some instances, certain Fv framework region (FR) residues of a human immunoglobulin are replaced with the corresponding residues in an antibody or fragment from a non-human species that has the desired specificity, affinity, and capability. The humanized antibody or antigen-binding fragment thereof can be further modified by the substitution of additional residues either in the Fv framework region and/or within the non-human CDR residues to refine and optimize antibody or antigen-binding fragment thereof specificity, affinity, and/or capability. In general, the humanized antibody or antigen-binding fragment thereof will comprise variable domains containing all or substantially all of the CDR regions that correspond to the non-human immunoglobulin whereas all or substantially all of the FR regions are those of a human immunoglobulin consensus sequence. The humanized antibody or antigen-binding fragment thereof can also comprise at least a portion of an immunoglobulin constant region or domain (Fc), typically that of a human immunoglobulin. Examples of methods used to generate humanized antibodies are described in U.S. Pat. No. 5,225,539; Roguska et al., Proc. Natl. Acad. Sci., USA, 91(3):969-73 (1994), and Roguska et al., Protein Eng. 9(10):895-904 (1996). In some aspects of the present disclosure, a “humanized antibody” is a resurfaced antibody.

The term “human” antibody or antigen-binding fragment thereof means an antibody or antigen-binding fragment thereof having an amino acid sequence derived from a human immunoglobulin gene locus, where such antibody or antigen-binding fragment is made using any technique known in the art. This definition of a human antibody or antigen-binding fragment thereof includes intact or full-length antibodies and fragments thereof.

“Binding affinity” generally refers to the strength of the sum total of non-covalent interactions between a single binding site of a molecule (e.g., an antibody or antigen-binding fragment thereof) and its binding partner (e.g., an antigen). Unless indicated otherwise, as used herein, “binding affinity” refers to intrinsic binding affinity which reflects a 1:1 interaction between members of a binding pair (e.g., antibody or antigen-binding fragment thereof and antigen). The affinity of a molecule X for its partner Y can generally be represented by the dissociation constant (KD). Affinity can be measured and/or expressed in a number of ways known in the art, including, but not limited to, equilibrium dissociation constant (KD), and equilibrium association constant (KA). The KD is calculated from the quotient of k_{off}/kₒₙ, whereas KA is calculated from the quotient of k_{off}/kₒₙ. Ko₁ refers to the association rate constant of, e.g., an antibody or antigen-binding fragment thereof to an antigen, and k_{off} refers to the dissociation of, e.g., an antibody or antigen-binding fragment thereof from an antigen. The kₒₙ and k_{off} can be determined by techniques known to one of ordinary skill in the art, such as BIAcore® or KinExA.

As used herein, an “epitope” is a term in the art and refers to a localized region of an antigen to which an antibody or antigen-binding fragment thereof can specifically bind. An epitope can be, for example, contiguous amino acids of a polypeptide (linear or contiguous epitope) or an epitope can, for example, come together from two or more non-contiguous regions of a polypeptide or polypeptides (conformational, non-linear, discontinuous, or non-contiguous epitope). In some aspects of the present disclosure, the epitope to which an antibody or antigen-binding fragment thereof specifically binds can be determined by, e.g., NMR spectroscopy, X-ray diffraction crystallography studies, ELISA assays, hydrogen/deuterium exchange coupled with mass spectrometry (e.g., liquid chromatography electrospray mass spectrometry), array-based oligo-peptide scanning assays, and/or mutagenesis mapping (e.g., site-directed mutagenesis mapping). For X-ray crystallography, crystallization can be accomplished using any of the known methods in the art (e.g., Giege R et al., (1994) Acta Crystallogr D Biol Crystallogr 50(Pt 4): 339-350; McPherson A (1990) Eur J Biochem 189: 1-23; Chayen NE (1997) Structure 5: 1269-1274; McPherson A (1976) J Biol Chem 251: 6300-6303). Crystal structures comprising antigen complexed with an antibody or antigen binding fragment can be studied using well known X-ray diffraction techniques and can be refined using computer software such as X-PLOR (Yale University, 1992, distributed by Molecular Simulations, Inc.; see, e.g., Meth Enzymol (1985) volumes 114 & 115, eds Wyckoff H W et al., U.S. 2004/0014194), and BUSTER (Bricogne G (1993) Acta Crystallogr D Biol Crystallogr 49(Pt 1): 37-60; Bricogne G (1997) Meth Enzymol 276A: 361-423, ed Carter CW; Roversi P et al., (2000) Acta Crystallogr D Biol Crystallogr 56(Pt 10): 1316-1323). Mutagenesis mapping studies can be accomplished using any method known to one of skill in the art. See, e.g., Champe M et al., (1995) J Biol Chem 270: 1388-1394 and Cunningham BC & Wells JA (1989) Science 244: 1081-1085 for a description of mutagenesis techniques, including alanine scanning mutagenesis techniques.

A polypeptide, antibody, polynucleotide, vector, cell, or composition which is “isolated” is a polypeptide, antibody, polynucleotide, vector, cell, or composition which is in a form not found in nature. Isolated polypeptides, antibodies, polynucleotides, vectors, cell or compositions include those which have been purified to a degree that they are no longer in a form in which they are found in nature. In some aspects of the present disclosure, an antibody, polynucleotide, vector, cell, or composition which is isolated is substantially pure. As used herein, “substantially pure” refers to material which is at least 50% pure (i.e., free from contaminants), at least 90% pure, at least 95% pure, at least 98% pure, or at least 99% pure.

The terms “polypeptide,” “peptide,” and “protein” are used interchangeably herein to refer to polymers of amino acids of any length. The polymer can be linear or branched, it can comprise modified amino acids, and it can be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. It is understood that, because the polypeptides of this disclosure are based upon antibodies, in some aspects of the present disclosure, the polypeptides can occur as single chains or associated chains.

As used herein, the term “pharmaceutical formulation” refers to a preparation which is in such form as to permit the biological activity of the active ingredient to be effective, and which contains no additional components which are unacceptably toxic to a subject to which the formulation would be administered. The formulation can be sterile. In some aspects, the formulation is suitable for therapeutic use in a human subject.

The terms “administer,” “administering,” “administration,” and the like, as used herein, refer to methods that can be used to enable delivery of a drug, e.g., an anti-TL1A antibody or antigen-binding fragment thereof to the desired site of biological action (e.g., intravenous administration). Administration techniques that can be employed with the agents and methods described herein are found in e.g., Goodman and Gilman, The Pharmacological Basis of Therapeutics, current edition, Pergamon; and Remington's, Pharmaceutical Sciences, current edition, Mack Publishing Co., Easton, Pa. and Matucci, A. et al., Respiratory Research, 19(1):154 (2018).

As used herein, the terms “combination” or “administered in combination” means that an antibody or antigen binding fragment thereof described herein can be administered with one or more additional therapeutic agents. In some aspects of a combination provided herein, an antibody or antigen binding fragment thereof can be administered with one or more additional therapeutic agents either simultaneously or sequentially. In some aspects, an antibody or antigen binding fragment thereof described herein can be administered with one or more additional therapeutic agent in the same composition or in different compositions.

The terms “subject” and “patient” are used interchangeably and include any animal. In some aspects, the subject is a mammal, including companion (e.g., cat, dog) and farm mammals (e.g., pig, horse, cow), as well as rodents, including mice, rabbits, and rats, guinea pigs, and other rodents. In some aspects, the subject is a non-human primates, such as cynomolgus monkeys. In some aspects, the subject is a human being.

The term “therapeutically effective amount” refers to an amount of a drug, e.g., an anti-TL1A antibody (e.g., TEV-48574) or antigen-binding fragment thereof, effective to treat a disease or disorder in a subject. Terms such as “treating,” “treatment,” “to treat,” “alleviating,” and “to alleviate” refer to utilizing an approach for obtaining beneficial or desired clinical results, including but not limited to an approach that achieves such beneficial or desired clinical results, wherein clinical results can include therapeutic measures that improve, cure, slow down, lessen symptoms of, and/or halt progression of a pathologic condition or disorder. Those in need of treatment can include those already diagnosed with or suspected of having the disorder.

“Specificity” in the context of antibody-antigen interactions is not necessarily an absolute designation but can constitute a relative term signifying the degree of selectivity of an antibody for an antigen-positive cell compared to an antigen-negative cell. Specificity of an antibody for an antigen-positive cell is mediated by the variable regions of the antibody, and usually by the complementarity determining regions (CDRs) of the antibody. A construct can have from about 100 to about 1000-fold specificity for antigen-positive cells compared to antigen-negative cells.

As used herein, the term “recombinant” includes the expression from genes made by genetic engineering or otherwise by laboratory manipulation.

As used herein, the term “TEV 48574” or “TEV-48574” refers to a highly potent, fully human immunoglobulin G (IgG) subclass 1 (IgG1) (lambda) monoclonal antibody (mAb) that binds with high affinity to human, cynomolgus monkey, and rat TL1A. TEV-48574 comprises the light chain of SEQ ID NO: 9 and the heavy chain of SEQ ID NO: 10. TEV-48574 is a blocking antibody that acts by competitively inhibiting the interaction of TL1A to its cognate signaling receptor, DR3. By competitively inhibiting TL1A binding to DR3, the antibody prevents activation of the DR3 signaling pathway. TEV-48574 also inhibits the binding of TL1A to DcR3 although TEV-48574 inhibits the TL1A-DR3 interaction over the TL1A-DcR3 interaction. TEV-48574 has shown anti-inflammatory and anti-fibrotic effects in colitis animal model. TEV-48574 was safe and well tolerated in the Phase 1 study TV48574-SAD-10126. TEV-48574 is disclosed as “320-587” in U.S. Pat. No. 10,138,296, which is herein incorporated by reference in its entirety.

As used herein, the term “TL1A” refers to the Tumor necrosis factor (TNF)-like ligand 1A, also known as TNF superfamily member 15 (TNFSF15) and vascular endothelial growth inhibitor (VEGI), is a member of the TNF superfamily, which is expressed by antigen presenting cells (including dendritic cells, B cells, and macrophages), CD4+ and CD8+ T cells, and endothelial cells. TL1A can be expressed on the cell surface or secreted as a soluble cytokine. The cognate signaling receptor for TL1A, Death Receptor 3 (DR3), is expressed by a variety of cells, including CD4+ and CD8+ T cells, NK cells, NKT cells, and FOXP3+ regulatory T (Treg) cells and type-2 and type-3 innate lymphoid cells (ILC2 and ILC3). TL1A can also bind a decoy receptor (DcR3), which is a competitive inhibitor of DR3. DcR3 also acts as a decoy receptor for Fas-ligand (Fas-L) and lymphotoxin-like inducible protein that competes with glycoprotein D for binding herpesvirus entry mediator on T cells (LIGHT). Accordingly, DcR3 is an important regulator of several signal transduction pathways.

As used in the present disclosure and claims, the singular forms “a,” “an,” and “the” include plural forms unless the context clearly dictates otherwise.

It is understood that wherever aspects of the present disclosure are described herein with the language “comprising,” otherwise analogous aspects described in terms of “consisting of” and/or “consisting essentially of” are also provided.

Unless specifically stated or obvious from context, as used herein, the term “or” is understood to be inclusive. The term “and/or” as used in a phrase such as “A and/or B” herein is intended to include both “A and B,” “A or B,” “A,” and “B.” Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

As used herein, the terms “about” and “approximately,” when used to modify a numeric value or numeric range, indicate that deviations of ±10% of the value or range remain within the intended meaning of the recited value or range. As is understood by one skilled in the art, reference to “about” a value or range herein includes (and describes) instances that are directed to that value or range per se. For example, description referring to “about X” includes description of “X.”

Any compositions or methods provided herein can be combined with one or more of any of the other compositions and methods provided herein.

Units, prefixes, and symbols are denoted in their Systéme International de Unites (SI) accepted form. Numeric ranges are inclusive of the numbers defining the range. The headings provided herein are not limitations of the various aspects of the disclosure, which can be had by reference to the specification as a whole. Accordingly, the terms defined immediately below are more fully defined by reference to the specification in its entirety.

### Pharmaceutical Compositions

The present disclosure provides pharmaceutical compositions comprising antibodies or antigen-binding fragments that bind to TL1A. The pharmaceutical compositions can comprise any of the antibodies or antigen binding fragments thereof described and/or exemplified herein and an acceptable carrier such as a pharmaceutically acceptable carrier. Suitable carriers include any media that does not interfere with the biological activity of the antibody or antigen-binding fragment thereof and is not toxic to a host to which it is administered. The pharmaceutical compositions can be formulated for administration to a subject in any suitable dosage form.

Pharmaceutical compositions suitable for administration to human patients are typically formulated for parenteral administration, e.g., in a liquid carrier, or suitable for reconstitution into liquid solution or suspension for intravenous or subcutaneous administration.

In general, such compositions typically comprise a pharmaceutically acceptable carrier. As used herein, the term “pharmaceutically acceptable” means approved by a government regulatory agency or listed in the U.S. Pharmacopeia or another generally recognized pharmacopeia for use in animals, particularly in humans. The term “carrier” refers to a diluent, adjuvant, excipient, or vehicle with which the compound is administered. Such pharmaceutical carriers can be sterile liquids. Water or aqueous solution saline and aqueous dextrose and glycerol solutions can be employed as carriers, particularly for injectable solutions. Liquid compositions for parenteral administration can be formulated for administration by injection or continuous infusion. Routes of administration by injection or infusion include intravenous and subcutaneous.

In some aspects, the pharmaceutical composition comprises: (a) about 100 mg/mL of a antibody or antigen-binding fragment thereof that specifically binds to TNF-like ligand 1A (TL1A); wherein the antibody or antigen-binding fragment thereof comprises: (i) a heavy chain variable region comprising the amino acid sequence of SEQ ID NO: 7 and a light chain variable region comprising the amino acid sequence of SEQ ID NO: 8; (ii) a heavy chain variable region CDR1 comprising the amino acid sequence of SEQ ID NO: 1, a heavy chain variable region CDR2 comprising the amino acid sequence of SEQ ID NO: 2, a heavy chain variable region CDR3 comprising the amino acid sequence of SEQ ID NO: 3, a light chain variable region CDR1 comprising the amino acid sequence of SEQ ID NO: 4, a light chain variable region CDR2 comprising the amino acid sequence of SEQ ID NO: 5, and a light chain variable region CDR3 comprising the amino acid sequence of SEQ ID NO: 6; or (iii) a heavy chain comprising the amino acid sequence of SEQ ID NO: 9 and a light chain comprising the amino acid sequence of SEQ ID NO: 10; (b) about 10 mM Histidine; (c) about 100 mM Arginine-Hydrochloride (Arg-HCl); (d) about 5% (w/v) Sucrose; and (e) about 0.02% (w/v) Polysorbate-80. In some aspects, the pharmaceutical formulation is lyophilized. In some aspects, the pharmaceutical formulation is liquid.

In some aspects, the pharmaceutical formulation comprises: (a) about 150 mg/mL of a antibody or antigen-binding fragment thereof that specifically binds to TNF-like ligand 1A (TL1A); wherein the antibody or antigen-binding fragment thereof comprises: (i) a heavy chain variable region comprising the amino acid sequence of SEQ ID NO: 7 and a light chain variable region comprising the amino acid sequence of SEQ ID NO: 8; (ii) a heavy chain variable region CDR1 comprising the amino acid sequence of SEQ ID NO: 1, a heavy chain variable region CDR2 comprising the amino acid sequence of SEQ ID NO: 2, a heavy chain variable region CDR3 comprising the amino acid sequence of SEQ ID NO: 3, a light chain variable region CDR1 comprising the amino acid sequence of SEQ ID NO: 4, a light chain variable region CDR2 comprising the amino acid sequence of SEQ ID NO: 5, and a light chain variable region CDR3 comprising the amino acid sequence of SEQ ID NO: 6; or (iii) a heavy chain comprising the amino acid sequence of SEQ ID NO: 9 and a light chain comprising the amino acid sequence of SEQ ID NO: 10; (b) about 10 mM Histidine; (c) about 100 mM Arginine-Hydrochloride (Arg-HCl); (d) about 5% (w/v) Sucrose; and (e) about 0.02% (w/v) Polysorbate-80. In some aspects, the pharmaceutical formulation is lyophilized. In some aspects, the pharmaceutical formulation is liquid.

In some aspects, the pharmaceutical formulation comprises: (a) about 100 mg/mL of a antibody or antigen-binding fragment thereof that specifically binds to TNF-like ligand 1A (TL1A); wherein the antibody or antigen-binding fragment thereof comprises: (i) a heavy chain variable region comprising the amino acid sequence of SEQ ID NO: 7 and a light chain variable region comprising the amino acid sequence of SEQ ID NO: 8; (ii) a heavy chain variable region CDR1 comprising the amino acid sequence of SEQ ID NO: 1, a heavy chain variable region CDR2 comprising the amino acid sequence of SEQ ID NO: 2, a heavy chain variable region CDR3 comprising the amino acid sequence of SEQ ID NO: 3, a light chain variable region CDR1 comprising the amino acid sequence of SEQ ID NO: 4, a light chain variable region CDR2 comprising the amino acid sequence of SEQ ID NO: 5, and a light chain variable region CDR3 comprising the amino acid sequence of SEQ ID NO: 6; or (iii) a heavy chain comprising the amino acid sequence of SEQ ID NO: 9 and a light chain comprising the amino acid sequence of SEQ ID NO: 10; (b) about 10 mM Histidine; (c) about 100 mM Arginine-Hydrochloride (Arg-HCl); (d) about 5% (w/v) Sucrose; and (e) about 0.02% (w/v) Polysorbate-80.

In some aspects, the pharmaceutical formulation comprises: (a) about 150 mg/mL of a antibody or antigen-binding fragment thereof that specifically binds to TNF-like ligand 1A (TL1A); wherein the antibody or antigen-binding fragment thereof comprises: (i) a heavy chain variable region comprising the amino acid sequence of SEQ ID NO: 7 and a light chain variable region comprising the amino acid sequence of SEQ ID NO: 8; (ii) a heavy chain variable region CDR1 comprising the amino acid sequence of SEQ ID NO: 1, a heavy chain variable region CDR2 comprising the amino acid sequence of SEQ ID NO: 2, a heavy chain variable region CDR3 comprising the amino acid sequence of SEQ ID NO: 3, a light chain variable region CDR1 comprising the amino acid sequence of SEQ ID NO: 4, a light chain variable region CDR2 comprising the amino acid sequence of SEQ ID NO: 5, and a light chain variable region CDR3 comprising the amino acid sequence of SEQ ID NO: 6; or (iii) a heavy chain comprising the amino acid sequence of SEQ ID NO: 9 and a light chain comprising the amino acid sequence of SEQ ID NO: 10; (b) about 10 mM Histidine; (c) about 100 mM Arginine-Hydrochloride (Arg-HCl); (d) about 5% (w/v) Sucrose; and (e) about 0.02% (w/v) Polysorbate-80.

In some aspects, the pharmaceutical formulations provided herein have a pH of 6.0±0.5 after storage at room temperature for 24 hours or at 2-8° C. for 24 hours, 72 hours, or 10 days. In some aspects, the pharmaceutical formulations provided herein have an osmolality of from 200 mOsm/kg to 500 mOsm/kg after storage at room temperature for 24 hours or at 2-8° C. for 24 hours, 72 hours, or 10 days. In some aspects, the pharmaceutical formulations provided herein have at least 99% antibody monomer content after storage at 2-8° C. for 24 hours, 72 hours, or 10 days. In some aspects, the pharmaceutical formulations provided herein have no significant change in charge heterogeneity profile after storage at 2-8° C. for 24 hours, 72 hours, or 10 days. In some aspects, the pharmaceutical formulations provided herein have no significant change in purity after storage at room temperature for 24 hours or at 2-8° C. for 24 hours, 72 hours, or 10 days. In some aspects, the pharmaceutical formulations provided herein have at least 90% purity after storage at room temperature for 24 hours or at 2-8° C. for 24 hours, 72 hours, or 10 days. In some aspects, the pharmaceutical formulations provided herein have no significant change in particle concentration after storage at room temperature for 24 hours or at 2-8° C. for 24 hours, 72 hours, or 10 days. In some aspects, the pharmaceutical formulations provided herein have no significant difference in visual appearance after storage at 2-8° C. for up to 24 months. In some aspects, the pharmaceutical formulations provided herein have no significant difference in visual appearance after storage at 2-8° C. for up to 36 months. In some aspects, the pharmaceutical formulations provided herein have no significant difference in protein concentration, osmolality or viscosity after storage at 2-8° C., 25° C. or 40° C. for up to 24 months. In some aspects, the pharmaceutical formulations provided herein have no significant difference in protein concentration, osmolality or viscosity after storage at 2-8° C., 25° C. or 40° C. for up to 36 months. In some aspects, the pharmaceutical formulations provided herein have ≥(more than or equal to) 95% monomer content, ≤ (less than or equal to) 5.0% dimer content, or no significant difference in low molecular weight species content after storage at 2-8° C. for up to 24 months. In some aspects, the pharmaceutical formulations provided herein have ≥ (more than or equal to) 95% monomer content, ≤ (less than or equal to) 5.0% dimer content, or no significant difference in low molecular weight species content after storage at 2-8° C. for up to 36 months. In some aspects, the pharmaceutical formulations provided herein have ≥ (more than or equal to) 90% purity after storage at 2-8° C. for up to 24 months. In some aspects, the pharmaceutical formulations provided herein have ≥ (more than or equal to) 90% purity after storage at 2-8° C. for up to 36 months. In some aspects, the pharmaceutical formulations provided herein have from 50% to 90% main species content, from 10% to 40% acidic species content, and/or from 0% to 20% basic species content after storage at 2-8° C. for up to 24 months. In some aspects, the pharmaceutical formulations provided herein have from 50% to 90% main species content, from 10% to 40% acidic species content, and/or from 0% to 20% basic species content after storage at 2-8° C. for up to 36 months. In some aspects, the pharmaceutical formulations provided herein have no significant difference in sub-visible particle content after storage at 2-8° C., 25° C., or 40° C. for up to 24 months. In some aspects, the pharmaceutical formulations provided herein have no significant difference in sub-visible particle content after storage at 2-8° C., 25° C., or 40° C. for up to 36 months. In some aspects, the pharmaceutical formulations provided herein have no significant difference in oxidation of methionine 81 and/or methionine 254 of TEV-48574, and/or deamidation of asparagine 317 of TEV-48574 after storage at 2-8° C., 25° C., or 40° C. for up to 24 months. In some aspects, the pharmaceutical formulations provided herein have no significant difference in oxidation of methionine 81 and/or methionine 254 of TEV-48574, and/or deamidation of asparagine 317 of TEV-48574 after storage at 2-8° C., 25° C., or 40° C. for up to 36 months. In some aspects, the pharmaceutical formulations provided herein have from 70% to 135% relative potency measured by enzyme-linked immunosorbent assay (ELISA) after storage at 2-8° C. for up to 24 months. In some aspects, the pharmaceutical formulations provided herein have from 70% to 135% relative potency measured by enzyme-linked immunosorbent assay (ELISA) after storage at 2-8° C. for up to 36 months. In some aspects, the pharmaceutical formulations provided herein have no significant difference in thermal stability after storage at 2-8° C., 25° C., or 40° C. for up to 6 months. In some aspects, the pharmaceutical formulations provided herein have no significant difference in thermal stability after storage at 2-8° C. for up to 36 months. In some aspects, the pharmaceutical formulations provided herein have no significant difference in secondary and/or tertiary protein structure after storage at 2-8° C., 25° C., or 40° C. for up to 3 months. In some aspects, the pharmaceutical formulations provided herein have no significant difference in secondary protein structure after storage at 2-8° C. for up to 36 months. In some aspects, the pharmaceutical formulations provided herein have no significant difference in concentration of Polysorbate-80 after storage at 2-8° C. for up to 24 months.

### Methods

The disclosure provides methods for treating a disease in a subject in need thereof, the method comprising administering to the subject any of the pharmaceutical formulations provided herein or any of the containers provided herein. In some aspects, the disease is a respiratory tract disease, a gastrointestinal disease, a skin disease, or an arthritis.

In some aspects, the respiratory tract disease is an asthma, a chronic obstructive pulmonary disease (COPD), a pulmonary fibrosis, a pulmonary sarcoidosis, an allergic rhinitis, or a cystic fibrosis.

In some aspects, the gastrointestinal is an inflammatory bowel disease, a Crohn's disease, a colitis, an ulcerative colitis, an eosinophilic esophagitis, or an irritable bowel syndrome.

In some aspects, the arthritis is a rheumatoid arthritis.

In some aspects, the skin disease is an atopic dermatitis, an eczema, or a scleroderma.

In some aspects, the subject is a human subject. In some aspects, the subject is a non-human primate such as a cynomolgus monkey. In some aspects, the subject is a non-human mammal such as a mouse, rat, guinea pig, cat, pig, rabbit, or dog.

### Anti-TL1A Antibodies

In some aspects, the formulations provided herein comprise anti-TL1A antibodies or antigen-binding fragments thereof. In some aspects, the antibody or antigen-binding fragment thereof inhibits the capability of TL1A to interact with DR3 and, in some aspects, also with DcR3 and, further inhibits the signalling induced by the interaction of TL1A with DR3. In some aspects, the antibody or antigen-binding fragment thereof has enhanced potency relative to antibody 320-179. In some aspects, the antibody or antigen-binding fragment thereof has enhanced affinity for TL1A relative to antibody 320-179.

In some aspects, the antibody or antigen-binding fragment thereof as disclosed herein, comprises a heavy chain variable region CDR1 comprising the amino acid sequence of SEQ ID NO: 1, a heavy chain variable region CDR2 comprising the amino acid sequence of SEQ ID NO: 2, a heavy chain variable region CDR3 comprising the amino acid sequence of SEQ ID NO: 3, a light chain variable region CDR1 comprising the amino acid sequence of SEQ ID NO: 4, a light chain variable region CDR2 comprising the amino acid sequence of SEQ ID NO: 5, and a light chain variable region CDR3 comprising the amino acid sequence of SEQ ID NO: 6. In some aspects, the antibody or antigen-binding fragment thereof is capable of inhibiting the interaction of TL1A with DR3.

In some aspects, the antibody or antigen-binding fragment thereof as disclosed herein, comprises a heavy chain variable region comprising the amino acid sequence of SEQ ID NO: 7. In some aspects, the antibody or antigen-binding fragment thereof comprises a light chain variable region comprising the amino acid sequence of SEQ ID NO: 8. In some aspects, the antibody or antigen-binding fragment thereof comprises a heavy chain variable region comprising the amino acid sequence of SEQ ID NO: 7 and a light chain variable region comprising the amino acid sequence of SEQ ID NO: 8.

In some aspects, the heavy chain variable region is joined to a heavy chain constant region. In some aspects, the heavy chain constant region is an IgG1, IgG2, or IgG4 heavy chain constant region. In some aspects, the heavy chain variable region of SEQ ID NO: 7 is joined to a human IgG1(ΔK) heavy chain constant region (e.g., SEQ ID NO: 14) such that the heavy chain comprises SEQ ID NO: 9. In some aspects, a heavy chain constant region provided herein does not comprise a C-terminal lysine. In some aspects, the light chain variable region of SEQ ID NO: 8 is joined to a lambda human light chain constant region (e.g., SEQ ID NO: 29) such that the light chain comprises SEQ ID NO: 10.

In some aspects, the antibody or antigen-binding fragment thereof as disclosed herein, is a monoclonal antibody or antigen-binding fragment thereof.

In some aspects, the antibody or antigen-binding fragment thereof as disclosed herein, is a recombinant antibody or antigen-binding fragment thereof. In some aspects, the recombinant antibody is a full length antibody. In some aspects, the recombinant antibody is a monoclonal antibody.

In some aspects, the antibody or antigen-binding fragment thereof as disclosed herein, binds to TL1A with enhanced affinity relative to a 320-179 anti-TL1A antibody, which comprises a heavy chain variable region comprising the amino acid sequence of SEQ ID NO: 11 and a light chain variable region comprising the amino acid sequence of SEQ ID NO: 12. In some aspects, the antibody or antigen-binding fragment thereof as disclosed herein, has enhanced potency relative to the 320-179 anti-TL1A antibody. The enhanced potency can be at least about 10-fold, at least about 12-fold, at least about 13-fold, at least about 15-fold, at least about 20-fold, at least about 25-fold, at least about 27-fold, at least about 40-fold greater potency, about 10-fold to about 40-fold, about 12-fold to about 40-fold, about 13-fold to about 40-fold, about 15-fold to about 40-fold, about 20-fold to about 40-fold, about 25-fold to about 40-fold, or about 27-fold to about 40-fold relative to the 320-179 anti-TL1A antibody. Fold-enhancement of potency can be determined according to a TL1A-induced caspase potency assay in TF-1 cells. See, e.g., U.S. Pat. No. 10,138,296. The 320-179 antibody had favourable biophysical properties, was a potent inhibitor of TL1A, and had a low predicted immunogenicity profile. U.S. Pat. No. 10,138,296 and U.S. Publ. No. 2014/0255302 are incorporated by reference herein in their entirety.

In some aspects, the antibody as disclosed herein, is TEV-48574, which is also referred to as the 320-587 antibody in U.S. Pat. No. 10,138,296 (VH is SEQ ID NO: 3 and VL is SEQ ID NO: 4 in that publication).

In some aspects, the antibody or antigen-binding fragment thereof as disclosed herein, comprises a heavy chain variable region comprising the amino acid sequence of SEQ ID NO: 7 and a light chain variable region comprising the amino acid sequence of SEQ ID NO: 8 and binds to TL1A with enhanced affinity relative to anti-TL1A 320-179 antibody, which comprises a heavy chain variable region comprising the amino acid sequence of SEQ ID NO: 11 and a light chain variable region comprising the amino acid sequence of SEQ ID NO: 12. The anti-TL1A 320-179 antibody has been previously described in U.S. Pat. No. 10,138,296 (VH is SEQ ID NO: 1 and VL is SEQ ID NO: 2 in that publication) and in U.S. Publ. No. 2014/0255302 (VH is SEQ ID NO: 186 and VL is SEQ ID NO: 199 in that publication). The 320-179 antibody had favourable biophysical properties, was a potent inhibitor of TL1A, and had a low predicted immunogenicity profile. U.S. Pat. No. 10,138,296 and U.S. Publ. No. 2014/0255302 are incorporated by reference herein in their entirety.

In some aspects, the antibody or antigen-binding fragment thereof as disclosed herein comprises a heavy chain variable region comprising the amino acid sequence of SEQ ID NO: 7 and a light chain variable region comprising the amino acid sequence of SEQ ID NO: 8 and has enhanced potency relative to the 320-179 anti-TL1A antibody. The enhanced potency can be at least about 10-fold, at least about 12-fold, at least about 13-fold, at least about 15-fold, at least about 20-fold, at least about 25-fold, at least about 27-fold, or at least about 40-fold greater potency relative to the 320-179 anti-TL1A antibody. Fold-enhancement of potency can be determined according to a TL1A-induced caspase potency assay in TF-1 human erythroleukemic cells (ATCC: CRL-2003). See, e.g., U.S. Pat. No. 10,138,296.

In some aspects, the antibody or antigen-binding fragment thereof as disclosed herein, is a full length antibody. In some aspects, the antibody as disclosed herein, is a monoclonal antibody.

In some aspects, the antibody or antigen-binding fragment thereof disclosed herein comprises a human IgG1 heavy chain constant region, a human IgG2 heavy chain constant region, or a human IgG4 heavy chain constant region, or any allotypes thereof. The human IgG1 heavy chain constant region can be selected from among human IgG1 (SEQ ID NO: 13), human IgG1 (ΔK) (SEQ ID NO: 14), human IgG1 252Y/254T/256E (SEQ ID NO: 15), human IgG1 252Y/254T/256E (ΔK) (SEQ ID NO: 16), human IgG1 L234A/L235A/G237A (SEQ ID NO: 17), human IgG1 L234A/L235A/G237A (ΔK) (SEQ ID NO: 18), human IgG1 L235A/G237A (SEQ ID NO: 19), and human IgG1 L235A/G237A (ΔK) (SEQ ID NO: 20). The human IgG2 heavy chain constant region can be selected from among human IgG2 with or without AK (SEQ ID NO: 21 and SEQ ID NO: 22) and human IgG2 A330S/P331S with or without (ΔK) (SEQ ID NO: 23 and SEQ ID NO: 24). The human IgG4 heavy chain constant region can be selected from among human IgG4 S228P (SEQ ID NO: 25), human IgG4 S228P (ΔK) (SEQ ID NO: 26), human IgG4 228P/252Y/254T/256E (SEQ ID NO: 27), and human IgG4 228P/252Y/254T/256E (ΔK) (SEQ ID NO: 28). It will be understood that an IgG4 heavy chain could be used without the stabilizing substitution S228P (e.g., IgG4 with YTE alone, IgG4 with YTE and ΔK, or IgG4 with ΔK alone).

In some aspects, the antibody or antigen-binding fragment thereof as disclosed herein, comprises a human lambda light chain constant region or an allotype thereof. The human light chain lambda constant region can comprise SEQ ID NO: 29.

In some aspects, the antibody or antigen-binding fragment thereof as disclosed herein, binds to human TL1A, and can bind to one or more of cynomolgus monkey TL1A, mouse TL1A, rat TL1A, guinea pig TL1A, cat TL1A, dog TL1A, pig TL1A, or rabbit TL1A. In some aspects, the antibody or antigen-binding fragment thereof can bind to TL1A of multiple different species, for example, if the epitope is shared. In some aspects, human TL1A comprises the amino acid sequence of SEQ ID NO: 34, SEQ ID NO: 35, or SEQ ID NO: 36. In some aspects, cynomolgus monkey TL1A comprises the amino acid sequence of SEQ ID NO: 37. In some aspects, mouse TL1A comprises the amino acid sequence of SEQ ID NO: 38. In some aspects, rat TL1A comprises the amino acid sequence of SEQ ID NO: 39. In some aspects, guinea pig TL1A comprises the amino acid sequence of SEQ ID NO: 40. In some aspects, cat TL1A comprises the amino acid sequence of SEQ ID NO: 41. In some aspects, pig TL1A comprises the amino acid sequence of SEQ ID NO: 42. In some aspects, rabbit TL1A comprises the amino acid sequence of SEQ ID NO: 43. In some aspects, dog TL1A comprises the amino acid sequence of SEQ ID NO: 44.

The antibody or antigen-binding fragment thereof as disclosed herein, has a binding affinity for an epitope on TL1A that includes an equilibrium dissociation constant (KD), which can be measured according to a kinetic exclusion assay, such as a KINEXA® assay (Sapidyne Instruments Inc., Boise, ID). The K_{D} for TL1A binding determined from a kinetic exclusion assay is less than about 1000 pM. In some aspects, the K_{D} for TL1A binding determined from a kinetic exclusion assay is less than about 500 pM, or less than about 400 pM, or less than about 300 pM, or less than about 200 pM. In some aspects, the K_{D} for TL1A binding determined from a kinetic exclusion assay is less than about 100 pM.

The K_{D} for TL1A binding determined from a kinetic exclusion assay can be from about 10 pM to about 100 pM. The K_{D} for TL1A binding determined from a kinetic exclusion assay can be from about 25 pM to about 75 pM. The K_{D} for TL1A binding determined from a kinetic exclusion assay can be from about 30 pM to about 60 pM. The K_{D} for TL1A binding determined from a kinetic exclusion assay can be from about 30 pM to about 50 pM. The K_{D} for TL1A binding determined from a kinetic exclusion assay can be from about 35 pM to about 50 pM. The K_{D} for TL1A binding determined from a kinetic exclusion assay can be from about 36 pM to about 46 pM. The K_{D} for TL1A binding determined from a kinetic exclusion assay can be from about 38 pM to about 44 pM. The K_{D} for TL1A binding determined from a kinetic exclusion assay can be from about 39 pM to about 43 pM. The K_{D} for TL1A binding determined from a kinetic exclusion assay can be from about 40 pM to about 45 pM. The K_{D} for TL1A binding determined from a kinetic exclusion assay can be from about 35 pM to about 42 pM. The K_{D} for TL1A binding determined from a kinetic exclusion assay can be about 40 pM. The K_{D} for TL1A binding determined from a kinetic exclusion assay can be about 41 pM. The K_{D} for TL1A binding determined from a kinetic exclusion assay can be about 42 pM. The kinetic exclusion assay can use the antibody molecule or TL1A molecule as the constant binding partner, and the other molecule as the titrant.

The antibody or antigen-binding fragment thereof as disclosed herein, is capable of binding to TL1A-positive cells. The antibody or antigen-binding fragment thereof as disclosed herein, can bind to a TL1A-positive cell with an EC₅₀ value of less than about 100 nM. less than about 75 nM, less than about 50 nM, less than about 30 nM, less than about 25 nM, less than about 20 nM, less than about 18 nM, less than about 15 nM, less than about 13 nM, or less than about 10 nM.

The antibody or antigen-binding fragment thereof as disclosed herein, can be monoclonal. In some aspects, the antibody or antigen-binding fragment thereof as disclosed herein, is a full length antibody comprising two heavy chains and two light chains. In some aspects, the antibody or antigen-binding fragment thereof as disclosed herein, comprises a derivative or fragment or portion of an antibody that retains the antigen-binding specificity, and also retains most or all of the affinity, of the full length antibody. For example, derivatives can comprise at least one variable region (either a heavy chain or light chain variable region). Derivatives can comprise at least two variable regions, e.g., at least one heavy chain variable region and at least one light chain variable region. Other examples of suitable antibody derivatives and fragments include, without limitation, antibodies with polyepitopic specificity, bispecific antibodies, multi-specific antibodies, diabodies, single-chain molecules, as well as FAb, F(Ab′)2, Fd, Fabc, and Fv molecules, single chain (Sc) antibodies, single chain Fv antibodies (scFv), individual antibody light chains, individual antibody heavy chains, fusions between antibody chains and other molecules, heavy chain monomers or dimers, light chain monomers or dimers, dimers consisting of one heavy and one light chain, and other multimers. Single chain Fv antibodies can be multi-valent. All antibody isotypes can be used to produce antibody derivatives, fragments, and portions. Antibody derivatives, fragments, and/or portions can be recombinantly produced and expressed by any cell type, prokaryotic or eukaryotic.

In a full-length antibody, each heavy chain is comprised of a heavy chain variable region (abbreviated herein as HCVR or VH) and a heavy chain constant region. The heavy chain constant region is comprised of three domains, CH1, CH2 and CH3. Each light chain is comprised of a light chain variable region (abbreviated herein as LCVR or VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. Typically, the antigen binding properties of an antibody are less likely to be disturbed by changes to FR sequences than by changes to the CDR sequences. Immunoglobulin molecules can be of any type (e.g., IgG, IgE, IgM, IgD, IgA and IgY), class (e.g., IgG1, IgG2, IgG3, IgG4, IgA1 and IgA2) or subclass.

In some aspects, the antibody or antigen-binding fragment thereof as disclosed herein, is fully human. Fully human antibodies are those where the whole molecule is human or otherwise of human origin, or includes an amino acid sequence identical to a human form of the antibody. Fully human antibodies include those obtained from a human V gene library, for example, where human genes encoding variable regions of antibodies are recombinantly expressed. Fully human antibodies can be expressed in other organisms (e.g., mice and xenomouse technology) or cells from other organisms transformed with genes encoding human antibodies. Fully human antibodies can nevertheless include amino acid residues not encoded by human sequences, e.g., mutations introduced by random or site directed mutations.

In some aspects, the antibody or antigen-binding fragment thereof as disclosed herein, comprises non-immunoglobulin derived protein frameworks. For example, reference can be made to Ku & Schutz, Proc. Natl. Acad. Sci. USA, 92:6552-6 (1995), which describes a four-helix bundle protein cytochrome b562 having two loops randomized to create CDRs, which have been selected for antigen binding.

In some aspects, the antibody or antigen-binding fragment thereof as disclosed herein, comprises post-translational modifications or moieties, which can impact antibody activity or stability. These modifications or moieties include, but are not limited to, methylated, acetylated, glycosylated, sulfated, phosphorylated, carboxylated, and amidated moieties and other moieties that are well known in the art. Moieties include any chemical group or combinations of groups commonly found on immunoglobulin molecules in nature or otherwise added to antibodies by recombinant expression systems, including prokaryotic and eukaryotic expression systems.

Examples of side chain modifications contemplated by the disclosure include modifications of amino groups such as by reductive alkylation by reaction with an aldehyde followed by reduction with NaBH₄; amidination with methylacetimidate; acylation with acetic anhydride; carbamoylation of amino groups with cyanate; trinitrobenzylation of amino groups with 2, 4, 6-trinitrobenzene sulphonic acid (TNBS); acylation of amino groups with succinic anhydride and tetrahydrophthalic anhydride; and pyridoxylation of lysine with pyridoxal-5-phosphate followed by reduction with NaBH₄.

The guanidine group of arginine residues can be modified by the formation of heterocyclic condensation products with reagents such as 2,3-butanedione, phenylglyoxal and glyoxal. The carboxyl group can be modified by carbodiimide activation via O-acylisourea formation followed by subsequent derivation, for example, to a corresponding amide. Sulfydryl groups can be modified by methods such as carboxymethylation with iodoacetic acid or iodoacetamide; performic acid oxidation to cysteic acid; formation of mixed disulfides with other thiol compounds; reaction with maleimide, maleic anhydride or other substituted maleimide; formation of mercurial derivatives using 4-chloromercuribenzoate, 4-chloromercuriphenylsulfonic acid, phenylmercury chloride, 2-chloromercuri-4-nitrophenol and other mercurials; carbamoylation with cyanate at alkaline pH. Tryptophan residues can be modified by, for example, oxidation with N-bromosuccinimide or alkylation of the indole ring with 2-hydroxy-5-nitrobenzyl bromide or sulfenyl halides. Tyrosine residues on the other hand, can be altered by nitration with tetranitromethane to form a 3-nitrotyrosine derivative. Modification of the imidazole ring of a histidine residue can be accomplished by alkylation with iodoacetic acid derivatives or N-carbethoxylation with diethylpyrocarbonate.

In some aspects, the antibody or antigen-binding fragment thereof as disclosed herein, includes one or more modifications that modulate serum half-life and biodistribution, including without limitation, modifications that modulate the antibody's interaction with the neonatal Fc receptor (FcRn), a receptor with a key role in protecting IgG from catabolism, and maintaining high serum antibody concentration. Serum half-life modulating modifications can occur in the Fc region of IgG1, IgG2, or IgG4, including the triple substitution of M252Y/S254T/T256E (the “YTE” substitutions, with numbering according to the EU numbering system (Edelman, G. M. et al., Proc. Natl. Acad. USA, 63:78-85 (1969)), as described in U.S. Pat. No. 7,083,784. Other substitutions can occur at positions 250 and 428, see e.g., U.S. Pat. No. 7,217,797, as well as at positions 307, 380 and 434, see, e.g., PCT Publ. No. WO 00/042072. Examples of constant domain amino acid substitutions which modulate binding to Fc receptors and subsequent function mediated by these receptors, including FcRn binding and serum half-life, are described in U.S. Publ. Nos. 2009/0142340, 2009/0068175, and 2009/0092599. Antibodies of any class can have the heavy chain C-terminal lysine omitted or removed to reduce heterogeneity (ΔK). The substitution of S228P (EU numbering) in the human IgG4 can stabilize antibody Fab-arm exchange in vivo (Labrin et al., Nature Biotechnology 27(8):767-73 (2009)), and this substitution can be present at the same time as the YTE and/or ΔK modifications.

The antibody or antigen-binding fragment thereof as disclosed herein, can be labelled, bound, or conjugated to any chemical or biomolecule moieties. Labelled antibodies can find use in therapeutic, diagnostic, or basic research applications. Such labels/conjugates can be detectable, such as fluorochromes, electrochemiluminescent probes, quantum dots, radiolabels, enzymes, fluorescent proteins, luminescent proteins, and biotin.

The antibody or antigen-binding fragment thereof as disclosed herein, can be derivatized by known protecting/blocking groups to prevent proteolytic cleavage or enhance activity or stability.

Administering the antibody or antigen-binding fragment thereof as disclosed herein, can comprise subcutaneously administering the antibody or antigen-binding fragment thereof. Accordingly, in some aspects, a formulation provided herein is formulated for subcutaneous administration. Administering can comprise intravenously administering the antibody or antigen-binding fragment thereof. Accordingly, in some aspects, a formulation provided herein is formulated for intravenous administration.

The disclosure further provides compositions for use in accordance with any method disclosed herein.

### Polynucleotides and Vectors

Polynucleotide sequences that encode the recombinant antibodies or antigen-binding fragments thereof and their subdomains (e.g., FRs and CDRs) are disclosed herein. Polynucleotides include, but are not limited to, RNA, DNA, cDNA, hybrids of RNA and DNA, and single, double, or triple stranded strands of RNA, DNA, or hybrids thereof. Polynucleotides can comprise a nucleic acid sequence encoding the heavy chain variable region and/or the light chain variable region of the antibody as described or exemplified herein. Complements of the polynucleotide sequences are also within the scope of the disclosure.

In some aspects, a polynucleotide can comprise a nucleic acid sequence encoding an antibody heavy chain variable region comprising the amino acid sequence of SEQ ID NO: 7. A polynucleotide encoding the amino acid sequence of SEQ ID NO: 7 can comprise the nucleic acid sequence of SEQ ID NO: 30 or SEQ ID NO: 31.

In some aspects, a polynucleotide can comprise a nucleic acid sequence encoding an antibody light chain variable region comprising the amino acid sequence of SEQ ID NO: 8. A polynucleotide encoding the amino acid sequence of SEQ ID NO: 8 can comprise the nucleic acid sequence of SEQ ID NO: 32 or SEQ ID NO: 33.

In some aspects, a polynucleotide can comprise a first nucleic acid sequence encoding an antibody heavy chain variable region and a second nucleic acid sequence encoding an antibody light chain variable region. A first nucleic acid sequence can encode an antibody heavy chain variable region comprising the amino acid sequence of SEQ ID NO: 7, and a second nucleic acid sequence can encode an antibody light chain variable region comprising the amino acid sequence of SEQ ID NO: 8. A first nucleic acid sequence encoding the amino acid sequence of SEQ ID NO: 7 can comprise the nucleic acid sequence of SEQ ID NO: 30 or SEQ ID NO: 31, and a second nucleic acid sequence encoding the amino acid sequence of SEQ ID NO: 8 can comprise the nucleic acid sequence of SEQ ID NO: 32 or SEQ ID NO: 33.

In some aspects, a polynucleotide can comprise a first nucleic acid sequence encoding an antibody heavy chain variable region and a second nucleic acid sequence encoding a heavy chain constant region. In some aspects, a polynucleotide comprises a first nucleic acid sequence encoding an antibody heavy chain variable region comprising the amino acid sequence of SEQ ID NO: 7 and a second nucleic acid sequence encoding an IgG1(ΔK) heavy chain constant region of SEQ ID NO: 14, for example, a polynucleotide comprising the nucleic acid sequence of SEQ ID NO: 31.

In some aspects, a polynucleotide can comprise a first nucleic acid sequence encoding an antibody light chain variable region and a second nucleic acid sequence encoding a light chain constant region. In some aspects, a polynucleotide comprises a first nucleic acid sequence encoding an antibody light chain variable region comprising the amino acid sequence of SEQ ID NO: 8 and a second nucleic acid sequence encoding a lambda light chain constant region of SEQ ID NO: 29, for example, a polynucleotide comprising the nucleic acid sequence of SEQ ID NO: 33.

Any of the polynucleotides described or exemplified herein can be comprised within a vector. Thus, vectors comprising polynucleotides are provided as part of the disclosure. The vectors can be expression vectors. Recombinant expression vectors containing a sequence encoding a polypeptide of interest are thus provided. The expression vector can contain one or more additional sequences, such as but not limited to regulatory sequences, a selection marker, a purification tag, or a polyadenylation signal. Such regulatory elements can include a transcriptional promoter, enhancers, mRNA ribosomal binding sites, or sequences that control the termination of transcription and translation.

Expression vectors, especially mammalian expression vectors, can include one or more nontranscribed elements, such as an origin of replication, a suitable promoter and enhancer linked to the gene to be expressed, other 5′ or 3′ flanking nontranscribed sequences, 5′ or 3′ nontranslated sequences (such as necessary ribosome binding sites), a polyadenylation site, splice donor and acceptor sites, or transcriptional termination sequences. An origin of replication that confers the ability to replicate in a specific host can also be incorporated.

The vectors can be used to transform any of a wide array of host cells well known to those of skill in the art, and host cells capable of expressing antibodies. Vectors include without limitation, plasmids, phagemids, cosmids, bacmids, bacterial artificial chromosomes (BACs), yeast artificial chromosomes (YACs), and baculovirus, as well as other bacterial, eukaryotic, yeast, and viral vectors. Suitable host cells include without limitation CHO cells, NS0 cells, HEK293 cells, or any eukaryotic stable cell line known or produced, and also include bacteria, yeast, and insect cells.

In some aspects, an antibody or antigen-binding fragment thereof provided herein was produced in a human embryonic kidney cell. In some aspects, an antibody or antigen-binding fragment thereof provided herein was produced in a HEK293 cell.

The antibody or antigen-binding fragment thereof can also be produced by hybridoma cells; methods to produce hybridomas being well known and established in the art.

### Kits and Containers

The disclosure also features containers comprising any pharmaceutical formulation described and exemplified herein. In some aspects, the container is a glass vial. In some aspects, the container is a glass vial having a fill volume of 3 mL. In some aspects, the container is a glass vial having a fill volume of 3 mL or less. In some aspects, the container is a glass vial having a fill volume of 2 mL or less.

The disclosure also features kits comprising any of the pharmaceutical formulations, or antibodies or antigen-binding fragments thereof described and exemplified herein. The kits can be used to supply pharmaceutical formulations, antibodies, antigen-binding fragments thereof, and other agents for use in diagnostic, basic research, or therapeutic methods, among others. In some aspects, the kits comprise any one or more of the pharmaceutical formulations, antibodies or antigen-binding fragments thereof described or exemplified herein and instructions for using the one or more pharmaceutical formulations, antibodies, or antigen-binding fragments thereof in a method of treating a disease in a subject in need thereof. The method can comprise administering to the subject any of the pharmaceutical formulations provided herein or any of the kits or containers provided herein. In some aspects, the disease is a respiratory tract disease, a gastrointestinal disease, a skin disease, or an arthritis.

In some aspects, the antibody or antigen-binding fragment thereof is formulated in a volume of 3 mL or less. In some aspects, the antibody or antigen-binding fragment thereof is formulated in a volume of 2 mL or less.

In some aspects, the respiratory tract disease is an asthma, a chronic obstructive pulmonary disease (COPD), a pulmonary fibrosis, a pulmonary sarcoidosis, an allergic rhinitis, or a cystic fibrosis.

In some aspects, the gastrointestinal is an inflammatory bowel disease, a Crohn's disease, a colitis, an ulcerative colitis, an eosinophilic esophagitis, or an irritable bowel syndrome.

In some aspects, the arthritis is a rheumatoid arthritis.

In some aspects, the skin disease is an atopic dermatitis, an eczema, or a scleroderma.

### Exemplary Aspects Provided Herein

In one aspect (Aspect 1; A1), provided herein is a pharmaceutical formulation, comprising: (a) 150 mg/mL of an antibody or antigen-binding fragment thereof that specifically binds to TNF-like ligand 1A (TL1A); wherein the antibody or antigen-binding fragment thereof comprises: (i) a heavy chain variable region comprising the amino acid sequence of SEQ ID NO: 7 and a light chain variable region comprising the amino acid sequence of SEQ ID NO: 8; (ii) a heavy chain variable region CDR1 comprising the amino acid sequence of SEQ ID NO: 1, a heavy chain variable region CDR2 comprising the amino acid sequence of SEQ ID NO: 2, a heavy chain variable region CDR3 comprising the amino acid sequence of SEQ ID NO: 3, a light chain variable region CDR1 comprising the amino acid sequence of SEQ ID NO: 4, a light chain variable region CDR2 comprising the amino acid sequence of SEQ ID NO: 5, and a light chain variable region CDR3 comprising the amino acid sequence of SEQ ID NO: 6; or (iii) a heavy chain comprising the amino acid sequence of SEQ ID NO: 9 and a light chain comprising the amino acid sequence of SEQ ID NO: 10; (b) 10 mM Histidine; (c) 100 mM Arginine-Hydrochloride (Arg-HCl); (d) 5% (w/v) Sucrose; and (e) 0.02% (w/v) Polysorbate-80.

In one aspect of A1, i.e., A2, the antibody or antigen-binding fragment of (i) or (ii) comprises an IgG1 constant region.

In one aspect of A1 or A2, i.e., A3, the pharmaceutical formulation is lyophilized.

In one aspect of A1 or A2, i.e., A4, the pharmaceutical formulation is liquid.

In one aspect of any one of A1-A4, i.e., A5, the pharmaceutical composition has a pH is 6.0±0.5 after storage at room temperature for 24 hours or at 2-8° C. for 24 hours, 72 hours, or 10 days.

In one aspect of any one of A1-A5, i.e., A6, the pharmaceutical composition has an osmolality from 200 mOsm/kg to 500 mOsm/kg after storage at room temperature for 24 hours or at 2-8° C. for 24 hours, 72 hours, or 10 days.

In one aspect of any one of A1-A6, i.e., A7, the pharmaceutical composition has at least 99% antibody monomer content after storage at 2-8° C. for 24 hours, 72 hours, or 10 days.

In one aspect of any one of A1-A7, i.e., A8, the pharmaceutical composition has no significant change in charge heterogeneity profile after storage at 2-8° C. for 24 hours, 72 hours, or 10 days.

In one aspect of any one of A1-A8, i.e., A9, the pharmaceutical composition has no significant change in purity after storage at room temperature for 24 hours or at 2-8° C. for 24 hours, 72 hours, or 10 days.

In one aspect of any one of A1-A8, i.e. A10, the pharmaceutical composition has at least 90% purity after storage at room temperature for 24 hours or at 2-8° C. for 24 hours, 72 hours, or 10 days.

In one aspect of any one of A1-A10, i.e., A1 l, the pharmaceutical composition has no significant change in particle concentration after storage at room temperature for 24 hours or at 2-8° C. for 24 hours, 72 hours, or 10 days.

In one aspect of any one of A1-A11, i.e., A12, the pharmaceutical composition has no significant difference in visual appearance after storage at 2-8° C. for up to 36 months.

In one aspect of any one of A1-A12, i.e., A13, the pharmaceutical composition has no significant difference in protein concentration, osmolality or viscosity after storage at 2-8° C., 25° C. or 40° C. for up to 36 months.

In one aspect of any one of A1-A13, i.e., A14, the pharmaceutical composition has ≥95% monomer content, ≤5.0% dimer content, or no significant difference in low molecular weight species content after storage at 2-8° C. for up to 36 months.

In one aspect of any one of A1-A14, i.e., A15, the pharmaceutical composition has ≥90% purity after storage at 2-8° C. for up to 36 months.

In one aspect of any one of A1-A15, i.e., A16, the pharmaceutical composition has from 50% to 90% main species content, from 10% to 40% acidic species content, and/or from 0% to 20% basic species content after storage at 2-8° C. for up to 36 months.

In one aspect of any one of A1-A16, i.e., A17, the pharmaceutical composition has no significant difference in sub-visible particle content after storage at 2-8° C., 25° C., or 40° C. for up to 36 months.

In one aspect of any one of A1-A17, i.e., A18, the pharmaceutical composition has no significant difference in oxidation of methionine 81 and/or methionine 254 of TEV-48574, and/or deamidation of asparagine 317 of TEV-48574 after storage at 2-8° C., 25° C., or 40° C. for up to 36 months.

In one aspect of any one of A1-A18, i.e., A19, the pharmaceutical composition has from 70% to 135% relative potency measured by enzyme-linked immunosorbent assay (ELISA) after storage at 2-8° C. for up to 36 months.

In one aspect of any one of A1-A19, i.e., A20, the pharmaceutical composition has no significant difference in thermal stability after storage at 2-8° C., 25° C., or 40° C. for up to 6 months.

In one aspect of any one of A1-A20, i.e., A21, the pharmaceutical composition has no significant difference in thermal stability after storage at 2-8° C. for up to 36 months.

In one aspect of any one of A1-A21, i.e., A22, the pharmaceutical composition has no significant difference in secondary and/or tertiary protein structure after storage at 2-8° C., 25° C., or 40° C. for up to 3 months.

In one aspect of any one of A1-A22, i.e., A23, the pharmaceutical composition has no significant difference in secondary protein structure after storage at 2-8° C. for up to 36 months.

In one aspect of any one of A1-A23, i.e., A24, the pharmaceutical composition has no significant difference in concentration of Polysorbate-80 after storage at 2-8° C. for up to 24 months.

In one aspect of any one of A1-A24, i.e., A25, the antibody or antigen-binding fragment thereof was produced in a Chinese hamster ovary cell.

In one aspect, i.e., A26, a container is provided comprising the pharmaceutical formulation of any one of A1-A25.

In one aspect of A26, i.e., A27, the container is a glass vial.

In one aspect of A27, i.e., A28, the container is a glass vial having a fill volume of 3 mL.

In one aspect, i.e., A29 provided herein is a method of treating a disease in a subject in need thereof, the method comprising administering to the subject the pharmaceutical formulation of any one of A1-A25 or the container of any one of A26-A28, optionally wherein the disease is a respiratory tract disease, a gastrointestinal disease, a skin disease, or an arthritis.

In one aspect of A29, i.e., A30, the respiratory tract disease is an asthma, a chronic obstructive pulmonary disease (COPD), a pulmonary fibrosis, a pulmonary sarcoidosis, an allergic rhinitis, or a cystic fibrosis.

In one aspect of A29, i.e., A31, the gastrointestinal disease is an inflammatory bowel disease, a Crohn's disease, a colitis, an ulcerative colitis, an eosinophilic esophagitis, or an irritable bowel syndrome.

In one aspect of A29, i.e., A32, the arthritis is a rheumatoid arthritis.

In one aspect of A29, i.e., A33, the skin disease is an atopic dermatitis, an eczema, or a scleroderma.

In one aspect of any one of A1-A25, i.e., A34, the formulation is for use in accordance with the method of any one of A29-A33.

In one aspect, i.e., B1, provided herein is a pharmaceutical formulation, comprising: (a) about 100 mg/mL to about 250 mg/mL of an antibody or antigen-binding fragment thereof that specifically binds to TNF-like ligand 1A (TL1A); wherein the antibody or antigen-binding fragment thereof comprises: a heavy chain variable region CDR1 comprising the amino acid sequence of SEQ ID NO: 1, a heavy chain variable region CDR2 comprising the amino acid sequence of SEQ ID NO: 2, a heavy chain variable region CDR3 comprising the amino acid sequence of SEQ ID NO: 3, a light chain variable region CDR1 comprising the amino acid sequence of SEQ ID NO: 4, a light chain variable region CDR2 comprising the amino acid sequence of SEQ ID NO: 5, and a light chain variable region CDR3 comprising the amino acid sequence of SEQ ID NO: 6; (b) about 5 mM to about 15 mM Histidine; (c) about 50 mM to about 150 mM Arginine-Hydrochloride (Arg-HCl); (d) about 2.5% (w/v) to about 7.5% (w/v) Sucrose; and (e) about 0.01% (w/v) to about 0.03% (w/v) Polysorbate-80.

In one aspect of B1, i.e., B2, the antibody or antigen-binding fragment thereof comprises a heavy chain variable region comprising the amino acid sequence of SEQ ID NO: 7 and a light chain variable region comprising the amino acid sequence of SEQ ID NO: 8.

In one aspect of B1 or B2, i.e., B3, the antibody or antigen-binding fragment comprises an IgG1 constant region.

In one aspect of any one of B1-B3, i.e., B4, the antibody or antigen-binding fragment thereof comprises a heavy chain comprising the amino acid sequence of SEQ ID NO: 9 and a light chain comprising the amino acid sequence of SEQ ID NO: 10.

In one aspect of any one of B1-B4, i.e., B5, the pharmaceutical formulation comprises about 100, about 150, about 200, about 225, or about 250 mg/mL of the antibody or antigen-binding fragment thereof.

In one aspect of any one of B1-B5, i.e., B6, the pharmaceutical formulation comprises about 5 mM, about 10 mM, or about 15 mM Histidine.

In one aspect of any one of B1-B6, i.e., B7 the pharmaceutical formulation comprises about 50 mM, about 100 mM, or about 150 mM Arginine-Hydrochloride (Arg-HCl).

In one aspect of any one of B1-B7, i.e., B8, the pharmaceutical formulation comprises about 2.5%, about 5%, or about 7.5% (w/v) Sucrose.

In one aspect of any one of B1-B8, i.e., B9, the pharmaceutical formulation comprises about 0.01%, about 0.02%, or about 0.03% (w/v) Polysorbate-80.

In one aspect of any one of B1-B9, i.e., B10, the pharmaceutical formulation is lyophilized.

In one aspect of any one of B1-B9, i.e., B11, the pharmaceutical formulation is liquid.

In one aspect of any one of B1-B11, i.e., B12, the pharmaceutical composition has a pH is 6.0±0.5 after storage at room temperature for 24 hours or at 2-8° C. for 24 hours, 72 hours, or 10 days.

In one aspect of any one of B1-B12, i.e., B13, the pharmaceutical composition has an osmolality from 200 mOsm/kg to 500 mOsm/kg after storage at room temperature for 24 hours or at 2-8° C. for 24 hours, 72 hours, or 10 days.

In one aspect of any one of B1-B13, i.e., B14, the pharmaceutical composition has at least 99% antibody monomer content after storage at 2-8° C. for 24 hours, 72 hours, or 10 days.

In one aspect of any one of B1-B14, i.e., B15, the pharmaceutical composition has no significant change in charge heterogeneity profile after storage at 2-8° C. for 24 hours, 72 hours, or 10 days.

In one aspect of any one of B1-B15, i.e., B16, the pharmaceutical composition has no significant change in purity after storage at room temperature for 24 hours or at 2-8° C. for 24 hours, 72 hours, or 10 days.

In one aspect of any one of B1-B15, i.e. B17, the pharmaceutical composition has at least 90% purity after storage at room temperature for 24 hours or at 2-8° C. for 24 hours, 72 hours, or 10 days.

In one aspect of any one of B1-B17, i.e., B18, the pharmaceutical composition has no significant change in particle concentration after storage at room temperature for 24 hours or at 2-8° C. for 24 hours, 72 hours, or 10 days.

In one aspect of any one of B1-B18, i.e., B19, the pharmaceutical composition has no significant difference in visual appearance after storage at 2-8° C. for up to 36 months.

In one aspect of any one of B1-B19, i.e., B20, the pharmaceutical composition has no significant difference in protein concentration, osmolality or viscosity after storage at 2-8° C., 25° C. or 40° C. for up to 36 months.

In one aspect of any one of B1-B20, i.e., B21, the pharmaceutical composition has ≥95% monomer content, ≤5.0% dimer content, or no significant difference in low molecular weight species content after storage at 2-8° C. for up to 36 months.

In one aspect of any one of B1-B21, i.e., B22, the pharmaceutical composition has ≥90% purity after storage at 2-8° C. for up to 36 months.

In one aspect of any one of B1-B22, i.e., B23, the pharmaceutical composition has from 50% to 90% main species content, from 10% to 40% acidic species content, and/or from 0% to 20% basic species content after storage at 2-8° C. for up to 36 months.

In one aspect of any one of B1-B23, i.e., B24, the pharmaceutical composition has no significant difference in sub-visible particle content after storage at 2-8° C., 25° C., or 40° C. for up to 36 months.

In one aspect of any one of B1-B24, i.e., B25, the pharmaceutical composition has no significant difference in oxidation of methionine 81 and/or methionine 254 of TEV-48574, and/or deamidation of asparagine 317 of TEV-48574 after storage at 2-8° C., 25° C., or 40° C. for up to 36 months.

In one aspect of any one of B1-B25, i.e., B26, the pharmaceutical composition has from 70% to 135% relative potency measured by enzyme-linked immunosorbent assay (ELISA) after storage at 2-8° C. for up to 36 months.

In one aspect of any one of B1-B26, i.e., B27, the pharmaceutical composition has no significant difference in thermal stability after storage at 2-8° C., 25° C., or 40° C. for up to 6 months.

In one aspect of any one of B1-B27, i.e., B28, the pharmaceutical composition has no significant difference in thermal stability after storage at 2-8° C. for up to 36 months.

In one aspect of any one of B1-B28, i.e., B29, the pharmaceutical composition has no significant difference in secondary and/or tertiary protein structure after storage at 2-8° C., 25° C., or 40° C. for up to 3 months.

In one aspect of any one of B1-B29, i.e., B30, the pharmaceutical composition has no significant difference in secondary protein structure after storage at 2-8° C. for up to 36 months.

In one aspect of any one of B1-B30, i.e., B31, the pharmaceutical composition has no significant difference in concentration of Polysorbate-80 after storage at 2-8° C. for up to 24 months.

In one aspect of any one of B1-B31, i.e., B32, the antibody or antigen-binding fragment thereof was produced in a Chinese hamster ovary cell.

In one aspect, i.e., B33, a container is provided comprising the pharmaceutical formulation of any one of B1-B32.

In one aspect of B33, i.e., B34, the container is a glass vial.

In one aspect of B34, i.e., B35, the container is a glass vial having a fill volume of 3 mL.

In one aspect of B33, i.e., B36, the container is a syringe, optionally wherein the syringe is a pre-filled syringe.

In one aspect, i.e., B37, provided herein is a method of treating a disease in a subject in need thereof, the method comprising administering to the subject the pharmaceutical formulation of any one of B1-B32 or the container of any one of B33-B36, optionally wherein the disease is a respiratory tract disease, a gastrointestinal disease, a skin disease, or an arthritis.

In one aspect of B37, i.e., B38, the respiratory tract disease is an asthma, a chronic obstructive pulmonary disease (COPD), a pulmonary fibrosis, a pulmonary sarcoidosis, an allergic rhinitis, or a cystic fibrosis.

In one aspect of B37, i.e., B39, the gastrointestinal disease is an inflammatory bowel disease, a Crohn's disease, a colitis, an ulcerative colitis, an eosinophilic esophagitis, or an irritable bowel syndrome.

In one aspect of B37, i.e., B40, the arthritis is a rheumatoid arthritis.

In one aspect of B37, i.e., B41, the skin disease is an atopic dermatitis, an eczema, or a scleroderma.

In one aspect of any one of B37-B41, i.e., B42, the pharmaceutical formulation is administered intravenously. In one aspect of any one of B37-B41, i.e., B43, the pharmaceutical formulation is administered subcutaneously.

In one aspect of any one of B1-B32 (i.e., B44), the formulation is for use in accordance with the method of any one of B37-B43.

The following examples are offered by way of illustration and not by way of limitation.

### EXAMPLES

### Example 1: Stability of Reconstituted TEV-48574 Drug Product Solution

The in-use stability of TEV-48574 reconstituted drug product was evaluated.

For this study, TEV-48574 was presented in lyophilized form at a protein concentration of 150 mg/mL in 10 mM Histidine, 5% (w/v) Sucrose, 100 mM arginine-hydrochloride (Arg-HCl), 0.02% (w/v) polysorbate-80 (PS-80) at pH 6.0. The lyophilized formulation was contained in 5 cc vials with 20 mm neck size. Vials were stored at 2-8° C. and brought to room temperature prior to reconstitution. Approximately 7 vials were reconstituted using 2.0 mL of sterile water for injection (WFI) in each vial to make stock solutions. The stock solutions were then diluted into different falcon tubes to concentrations of 50, 20 and 5 mg/mL using a formulation buffer of 10 mM Histidine, 5% (w/v) Sucrose, 100 mM Arg-HCl, and 0.02% (w/v) PS80, at pH 6.0. After dilution, 4 mL samples were subjected to incubation for 24 hours, 72 hours, and 10 days at 2-8° C. protected from light, and for 24 hours at room temperature under normal light conditions. Table 1 shows the specific time points and conditions tested.

TABLE 1
Conditions and Time Points Tested for
Reconstituted TEV-48574 Stability Study

| TEV-48574 | | | 24 | 72 | 10 |
|---|---|---|---|---|---|
| concentrations | Conditions | T0 | hours | hours | days |
| 50 mg/mL | 2-8° C. (protected from light) | X | X | X | X |
| | Room temperature (Normal | | X | | |
| | Lighting conditions) | | | | |
| | Sample volume required (mL) | 4 | 8 | 4 | 4 |
| 20 mg/mL | 2-8° C. (protected from light) | X | X | X | X |
| | Room temperature (Normal | | X | | |
| | Lighting conditions) | | | | |
| | Sample volume required (mL) | 4 | 8 | 4 | 4 |
| 5 mg/mL | 2-8° C. (protected from light) | X | X | X | X |
| | Room temperature (Normal | | X | | |
| | Lighting conditions) | | | | |
| | Sample volume required (mL) | 4 | 8 | 4 | 4 |

Note:
T0 = time zero

At the end of each time point and condition, samples were analyzed by visual appearance, pH, osmolality, protein concentration, size exclusion chromatography (SEC), capillary sodium dodecyl sulfate gel electrophoresis (cSDS), capillary isoelectric focusing (cIEF), and sub-visible particle analysis using micro flow imaging (MFJ).

As shown in Table 2, no significant difference was seen in reconstituted samples upon dilution with respect to visual appearance, pH, osmolality and protein concentration. Also, pH and osmolality values of the samples were within the limits of 6.0±0.5 pH and 200-500 mOsm/kg, respectively.

TABLE 2
Visual Appearance, pH, Osmolality and Protein
Concentration of Reconstituted Product

| Condition and | | Osmolality | Measured Protein | | |
|---|---|---|---|---|---|
| time point | pH | (mOsm/kg) | concentration (mg/mL) | | Appearance |
| 5 mg/mL, T0 | 6.36 | 362 | 5.8 | mg/mL | Liquid, C, L, F |
| 5 mg/mL, RT, 24 hours | 6.35 | 360 | 5.7 | mg/mL | Liquid, C, L, F |
| 5 mg/mL, 2-8° C., 24 hours | 6.34 | 358 | 4.5 | mg/mL | Liquid, C, L, F |
| 5 mg/mL, 2-8° C., 72 hours | 6.34 | 358 | 5.6 | mg/mL | Liquid, C, L, F |
| 5 mg/mL, 2-8° C., 10 days | 6.36 | 364 | 5.7 | mg/mL | Liquid, C, L, F |
| 20 mg/mL, T0 | 6.38 | 370 | 22.0 | mg/mL | Liquid, C, L, F |
| 20 mg/mL, RT, 24 hours | 6.34 | 363 | 22.2 | mg/mL | Liquid, C, L, F |
| 20 mg/mL, 2-8° C., 24 hours | 6.36 | 364 | 21.9 | mg/mL | Liquid, C, L, F |
| 20 mg/mL, 2-8° C., 72 hours | 6.35 | 363 | 22.3 | mg/mL | Liquid, C, L, F |
| 20 mg/mL, 2-8° C., 10 days | 6.39 | 368 | 22.2 | mg/mL | Liquid, C, L, F |
| 50 mg/mL, T0 | 6.37 | 374 | 54.4 | mg/mL | Liquid, C, L, F |
| 50 mg/mL, RT, 24 hours | 6.36 | 375 | 54.3 | mg/mL | Liquid, C, L, F |
| 50 mg/mL, 2-8° C., 24 hours | 6.37 | 372 | 55.2 | mg/mL | Liquid, C, L, F |
| 50 mg/mL, 2-8° C., 72 hours | 6.35 | 377 | 55.6 | mg/mL | Liquid, C, L, F |
| 50 mg/mL, 2-8° C., 10 days | 6.36 | 375 | 54.8 | mg/mL | Liquid, C, L, F |

Note:
T0 = time zero;
RT = room temperature;
C = clear solution,
L = colorless,
F = free from visible particles

SEC analysis showed that the % monomer level of antibody in the samples did not have any significant change over the duration of the stability evaluation. See Table 3. These results indicate that storage of the reconstituted drug product upon dilution at 2-8° C. for up to 10 days did not have any effect on the % monomer level upon incubation.

TABLE 3
Percent Monomer Level of Reconstituted Product

| Condition and | | | % Main |
|---|---|---|---|
| time point | % HMW | % Dimer | peak |
| 5 mg/mL, T0 | 0.7 | 0.1 | 99.3 |
| 5 mg/mL, RT, 24 hours | 0.7 | 0.1 | 99.3 |
| 5 mg/mL, 2-8° C., 24 hours | 0.6 | 0.1 | 99.3 |
| 5 mg/mL, 2-8° C., 72 hours | 0.7 | 0.1 | 99.3 |
| 5 mg/mL, 2-8° C., 10 days | 0.6 | 0.1 | 99.3 |
| 20 mg/mL, T0 | 0.7 | 0.1 | 99.3 |
| 20 mg/mL, RT, 24 hours | 0.6 | 0.1 | 99.3 |
| 20 mg/mL, 2-8° C., 24 hours | 0.7 | 0.1 | 99.2 |
| 20 mg/mL, 2-8° C., 72 hours | 0.7 | 0.1 | 99.3 |
| 20 mg/mL, 2-8° C., 10 days | 0.7 | 0.1 | 99.2 |
| 50 mg/mL, T0 | 0.7 | 0.1 | 99.3 |
| 50 mg/mL, RT, 24 hours | 0.7 | 0.1 | 99.2 |
| 50 mg/mL, 2-8° C., 24 hours | 0.7 | 0.1 | 99.2 |
| 50 mg/mL, 2-8° C., 72 hours | 0.7 | 0.1 | 99.2 |
| 50 mg/mL, 2-8° C., 10 days | 0.7 | 0.1 | 99.2 |

Note:
T0 = time zero;
RT = room temperature;
HMW = high molecular weight species

cIEF analysis showed that dilution of reconstituted drug product and its incubation at 2-8° C. for up to 10 days does not have any significant impact on the charge heterogeneity profile of TEV-48574. See Table 4.

TABLE 4
cIEF Analysis of Reconstituted Product

| Condition and | % Acidic | % Main | % Basic |
|---|---|---|---|
| time point | species | peak | species |
| 5 mg/mL, T0 | 22.8 | 72.3 | 4.9 |
| 5 mg/mL, RT, 24 hours | 23.1 | 71.8 | 5.1 |
| 5 mg/mL, 2-8° C., 24 hours | 23.1 | 71.9 | 4.9 |
| 5 mg/mL, 2-8° C., 72 hours | 24.2 | 70.1 | 4.7 |
| 5 mg/mL, 2-8° C., 10 days | 23.0 | 72.6 | 4.4 |
| 20 mg/mL, T0 | 20.2 | 75.3 | 4.5 |
| 20 mg/mL, RT, 24 hours | 19.7 | 76.0 | 4.3 |
| 20 mg/mL, 2-8° C., 24 hours | 19.9 | 75.1 | 5.1 |
| 20 mg/mL, 2-8° C., 72 hours | 20.2 | 75.3 | 4.4 |
| 20 mg/mL, 2-8° C., 10 days | 19.6 | 75.8 | 4.5 |
| 50 mg/mL, T0 | 20.1 | 75.0 | 4.9 |
| 50 mg/mL, RT, 24 hours | 19.5 | 76.2 | 4.2 |
| 50 mg/mL, 2-8° C., 24 hours | 19.8 | 75.7 | 4.5 |
| 50 mg/mL, 2-8° C., 72 hours | 19.7 | 76.1 | 4.3 |
| 50 mg/mL, 2-8° C., 10 days | 19.0 | 76.8 | 4.2 |

Note:
T0 = time zero

As shown in Table 5, cSDS analysis revealed that reconstituted drug product purity was >9000, and that dilution and incubation of reconstituted drug product at different conditions for various time points had no significant impact on purity.

TABLE 5
cSDS Analysis of Reconstituted Product

| | | | | R-cSDS |
|---|---|---|---|---|
| | NR-cSDS | | | % Reduced |
| | % | | | purity |
| Condition and | frag- | % Main | % | (% HC + |
| time point | ment | peak | HMW | % LC) |
| 5 mg/mL, T0 | 1.7 | 98.3 | 0.0 | 96.3 |
| 5 mg/mL, RT, 24 hours | 0.0 | 100.0 | 0.0 | 100.0 |
| 5 mg/mL, 2-8° C., 24 hours | 1.8 | 98.2 | 0.0 | 95.1 |
| 5 mg/mL, 2-8° C., 72 hours | 0.0 | 100.0 | 0.0 | 100.0 |
| 5 mg/mL, 2-8° C., 10 days | 0.0 | 100.0 | 0.0 | 100.0 |
| 20 mg/mL, T0 | 1.6 | 94.5 | 3.9 | 94.1 |
| 20 mg/mL, RT, 24 hours | 1.9 | 95.2 | 2.9 | 93.2 |
| 20 mg/mL, 2-8° C., 24 hours | 3.6 | 94.2 | 2.2 | 93.0 |
| 20 mg/mL, 2-8° C., 72 hours | 1.8 | 98.2 | 0.0 | 93.5 |
| 20 mg/mL, 2-8° C., 10 days | 1.9 | 96.8 | 1.3 | 93.5 |
| 50 mg/mL, T0 | 3.8 | 92.6 | 3.7 | 92.9 |
| 50 mg/mL, RT, 24 hours | 1.6 | 95.1 | 3.4 | 93.3 |
| 50 mg/mL, 2-8° C., 24 hours | 1.5 | 96.0 | 2.5 | 92.6 |
| 50 mg/mL, 2-8° C., 72 hours | 3.4 | 93.9 | 2.7 | 93.5 |
| 50 mg/mL, 2-8° C., 10 days | 2.1 | 96.4 | 1.5 | 93.5 |

Note:
NR = nonreducing;
R = reducing;
HC = heavy chain;
LC = light chain;
HMW = high molecular weight species;
T0 = time zero;
RT = room temperature

MFI analysis showed that 50 mg/mL samples contained a slightly higher number of sub-visible particles. See Table 6. However, no significant increase in particle concentration was observed overall for different concentration samples when comparing initial time point samples to later time point samples.

TABLE 6
Particle Size Concentration of Reconstituted Product

| | Particle concentration (#/mL) | | | |
|---|---|---|---|---|
| Condition and time point | ≥2 um | ≥5 um | ≥10 um | ≥25 um |
| 5 mg/mL, T0 | 1164 | 306 | 76 | 4 |
| 5 mg/mL, RT, 24 hours | 843 | 197 | 45 | 5 |
| 5 mg/mL, 2-8° C., 24 hours | 296 | 46 | 10 | 3 |
| 5 mg/mL, 2-8° C., 72 hours | 1132 | 317 | 80 | 9 |
| 5 mg/mL, 2-8° C., 10 days | 4981 | 1322 | 253 | 10 |
| 20 mg/mL, T0 | 869 | 95 | 18 | 1 |
| 20 mg/mL, RT, 24 hours | 814 | 72 | 5 | 1 |
| 20 mg/mL, 2-8° C., 24 hours | 808 | 84 | 8 | 0 |
| 20 mg/mL, 2-8° C., 72 hours | 1199 | 197 | 40 | 7 |
| 20 mg/mL, 2-8° C., 10 days | 1537 | 289 | 60 | 7 |
| 50 mg/mL, T0 | 1706 | 178 | 21 | 4 |
| 50 mg/mL, RT, 24 hours | 1459 | 152 | 25 | 2 |
| 50 mg/mL, 2-8° C., 24 hours | 2812 | 568 | 124 | 9 |
| 50 mg/mL, 2-8° C., 72 hours | 3240 | 682 | 159 | 18 |
| 50 mg/mL, 2-8° C., 10 days | 2022 | 356 | 56 | 6 |

Note:
T0 = time zero;
RT = room temperature

Taken together, these results show that reconstituted TEV-48574 drug product is stable upon dilution over a period of 10 days upon storage at 2-8° C. protected from light and for 24 hours at room temperature under normal light conditions.

### Example 2: Stability of TEV-48574 Liquid Formulations at 100 mg/ml and 150 mg/ml

The purpose of this study was to evaluate the long term stability of liquid and lyophilized TEV-48574 formulations. Three formulations were tested: a lyophilized form containing 100 mg/mL of drug product in 10 mM Histidine, 5% (w/v) Sucrose, 100 mM Arg-HCl, 0.02% (w/v) PS-80 at pH 6.0 (Formulation 1), and two liquid forms containing 100 mg/mL or 150 mg/mL of drug product in 10 mM Histidine, 5% (w/v) Sucrose, 100 mM Arg-HCl, 0.02% (w/v) PS-80 at pH 6.0 (Formulations 2 and 3, respectively). The stability of these formulations was evaluated under the following conditions in 5 cc vials (Type I glass): “standard” condition (2-8° C.), “accelerated” condition (25±2° C./60±5% relative humidity (RH)), and “stressed” condition (40±2° C./75±5% RH). Furthermore, the stability of Formulation 3 was evaluated under the same conditions but with the formulation filled into 2.25 mL Nipro pre-filled syringes (Nipro PFS) fitted with West plunger-stoppers. The impact of these conditions on several product quality attributes was tested for the formulations.

### Visual Appearance, Protein Concentration, Osmolality and Viscosity

The results of a visual appearance, protein concentration and osmolality analysis for Formulations 1-3 are shown in Tables 7-10. At standard storage conditions of 2-8° C., no significant difference across the formulations was observed. At intermittent time points, few visible particles were observed. However, this could be related to the development nature of the study where drug product was manually filled into vials. Furthermore, these particles cannot be product-related as they did not increase over time and were not detected consistently in all the samples across different time points. For visual appearance at stressed conditions of 40° C., the solutions appeared slightly yellow at later time points. No significant differences were observed between Formulation 3 filled into Nipro PFS compared to Formulation 3 stored in glass vials up to 24 months with respect to visual appearance, protein concentration, osmolality, and viscosity (data not shown), indicating that these attributes of the formulation are not affected by contact with the Nipro PFS.

The measured protein concentration for all three formulations at standard (2-8° C.), accelerated (25° C.), and stressed (40° C.) conditions were close to the nominal concentration expected and did not vary over time. Similar trends were observed for osmolality and viscosity measured at standard storage conditions at time zero (T0), 24 months (24M), and 36 months (36M) with no significant difference observed over time.

TABLE 7
Visual Appearance and Protein Concentration Results at 2-8° C.

| | | | | | Protein concentration | | |
|---|---|---|---|---|---|---|---|
| Time | | Visual appearance | | | (mg/mL) | | |
| Point | | F1 | F2 | F3 | F1 | F2 | F3 |
| T0 | | L; S; FFVP | L; S; 1-3 particle | L; S; 1 particle | 103.1 | 106.2 | 153.4 |
| | | | observed | observed | | | |
| 1 | M | L; S; FFVP | L; S; 1 particle | L; S; FFVP | 99.7 | 106.8 | 154.2 |
| | | | observed | | | | |
| 3 | M | L; S; FFVP | L; S; FFVP | L; S; FFVP | 99.5 | 107.1 | 155.7 |
| 6 | M | L; S; FFVP | L; S; 1 Fibrous | L; S; FFVP | 99.3 | 105.5 | 148.8 |
| | | | particle detected | | | | |
| 9 | M | L; S; FFVP | L; S; FFVP | L; S; FFVP | 93.4 | 105.2 | 152.1 |
| 12 | M | NT | L; S; FFVP | L; S; FFVP | 102.2 | 106.9 | 149.6 |
| 18 | M | L; S; FFVP | L; S; 1 particle | L; S; FFVP | 94.4 | 102.9 | 153.1 |
| | | | observed | | | | |
| 24 | M | L; S; FFVP | L; S; FFVP | S; SY; FFVP | 97.4 | 104.9 | 148.8 |
| 36 | M | L; S; FFVP | L; S; FFVP | L; S; FFVP | 101.1 | 106.3 | 153.2 |

T0: Time zero;
M: Month(s);
F1: Formulation 1;
F2: Formulation 2;
F3: Formulation 3;
S: Slightly opalescent;
L: Colorless;
FFVP: Free from visible particles;
SY: Slightly yellow;
NT: Not tested

TABLE 8
Osmolality and Viscosity Results at 2-8° C.

| Time | Osmolality (mOsm/kg) | | | Viscosity (cP) measured at 20° C. | | |
|---|---|---|---|---|---|---|
| Point | F1 | F2 | F3 | F1 | F2 | F3 |
| T0 | 379 | 390 | 434 | 3.4 | 3.5 | 7.8 |
| 24 M | 360 | 379 | 425 | 3.3 | 3.5 | 7.6 |
| 36 M | 401 | 399 | 462 | NT | NT | NT |

T0: Time zero;
M: Month(s);
F1: Formulation 1;
F2: Formulation 2;
F3: Formulation 3;
NT: Not tested

TABLE 9
Visual Appearance and Protein Concentration Results at 25° C.

| | | | | | Protein concentration | | |
|---|---|---|---|---|---|---|---|
| Time | | Visual appearance | | | (mg/mL) | | |
| point | | F1 | F2 | F3 | F1 | F2 | F3 |
| T0 | | L; S; FFVP | L; S; 1-3 | L; S; 1 | 103.1 | 106.2 | 153.4 |
| | | | particles | particle | | | |
| | | | observed | observed | | | |
| 2 | WK | L; S; FFVP | L; S; FFVP | L; S; 1-2 | 100.8 | 106.7 | 153.9 |
| | | | | particles | | | |
| 1 | M | L; S; FFVP | L; S; 1 | L; S; FFVP | 101.5 | 107.2 | 155 |
| | | | particle | | | | |
| | | | observed | | | | |
| 2 | M | L; S; FFVP | L; S; FFVP | L; S; FFVP | 101.2 | 106.8 | 153.6 |
| 3 | M | L; S; Few | L; S; FFVP | L; S; FFVP | 99 | 106.2 | 153.5 |
| | | Particles | | | | | |
| 6 | M | L; S; FFVP | L; S; FFVP | L; S; FFVP | 97.7 | 106.8 | 151.7 |
| 9 | M | L; S; FFVP | L; S; FFVP | L; S; 1 | 97.4 | 106.4 | 156.8 |
| | | | | particle | | | |
| | | | | observed | | | |
| 12 | M | NT | L; S; 1 | L; S; 1-2 | 101 | 107.7 | 153 |
| | | | particle | particles | | | |
| | | | observed | observed | | | |

T0: Time zero;
WK: Week(s);
M: Month(s);
F1: Formulation 1;
F2: Formulation 2;
F3: Formulation 3;
S: Slightly opalescent;
L: Colorless;
FFVP: Free from visible particles;
SY: Slightly yellow;
NT: Not tested

TABLE 10
Visual Appearance and Protein Concentration Results at 40° C.

| | | | | | Protein concentration | | |
|---|---|---|---|---|---|---|---|
| Time | | Visual appearance | | | (mg/mL) | | |
| point | | F1 | F2 | F3 | F1 | F2 | F3 |
| T0 | | L; S; FFVP | L; S; 1-3 | particle | 103.1 | 106.2 | 153.4 |
| | | | particle observed | L; S; 1 | | | |
| | | | | observed | | | |
| 2 | WK | L; S; FFVP | L; S; 1 particle | L; S; FFVP | 100.5 | 105.8 | 153.1 |
| | | | observed | | | | |
| 1 | M | L; S; 1 | L; S; 1 particle | L; S; FFVP | 99.9 | 106.7 | 154.9 |
| | | particle | observed | | | | |
| | | observed | | | | | |
| 2 | M | L; S; FFVP | L; S; FFVP | S; SY; | 100.9 | 106.7 | 152.5 |
| | | | | FFVP | | | |
| 3 | M | L; S; Few | L; S; FFVP | S; SY; Few | 99.1 | 106.7 | 154.5 |
| | | particles | | particles | | | |
| | | | | were | | | |
| | | | | observed | | | |
| 6 | M | L; S; FFVP | S; SY; FFVP | S; SY; | 95.4 | 106.1 | 153.0 |
| | | | | FFVP | | | |

T0: Time zero;
WK: Week(s);
M: Month(s);
F1: Formulation 1;
F2: Formulation 2;
F3: Formulation 3;
S: Slightly opalescent;
L: Colorless;
FFVP: Free from visible particles;
SY: Slightly yellow;
NT: Not tested

### Size Exclusion Chromatography (SEC)

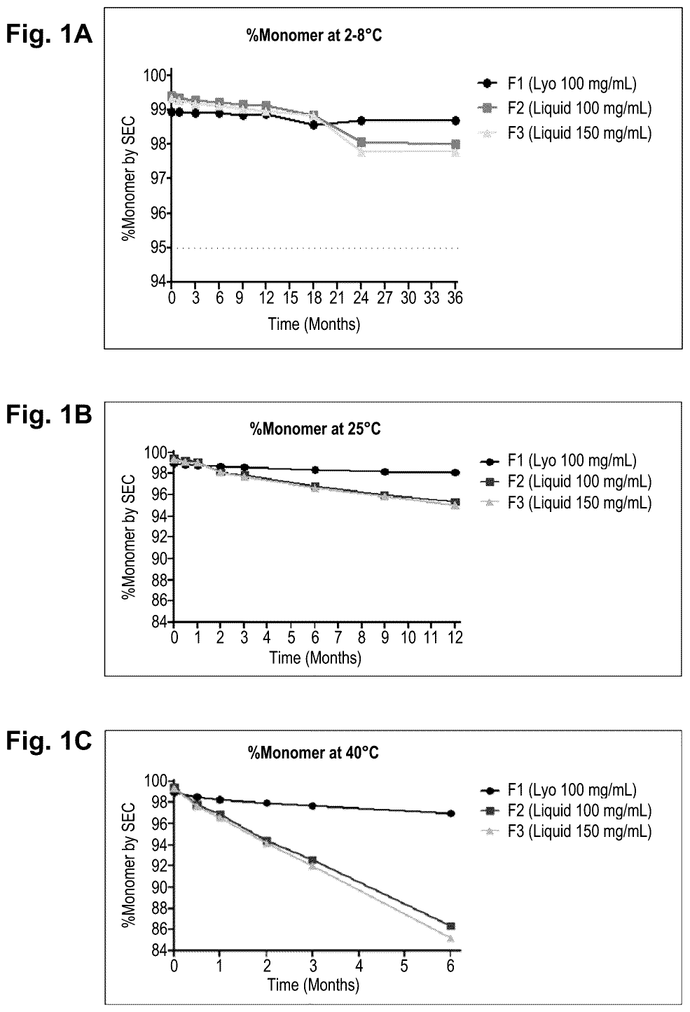

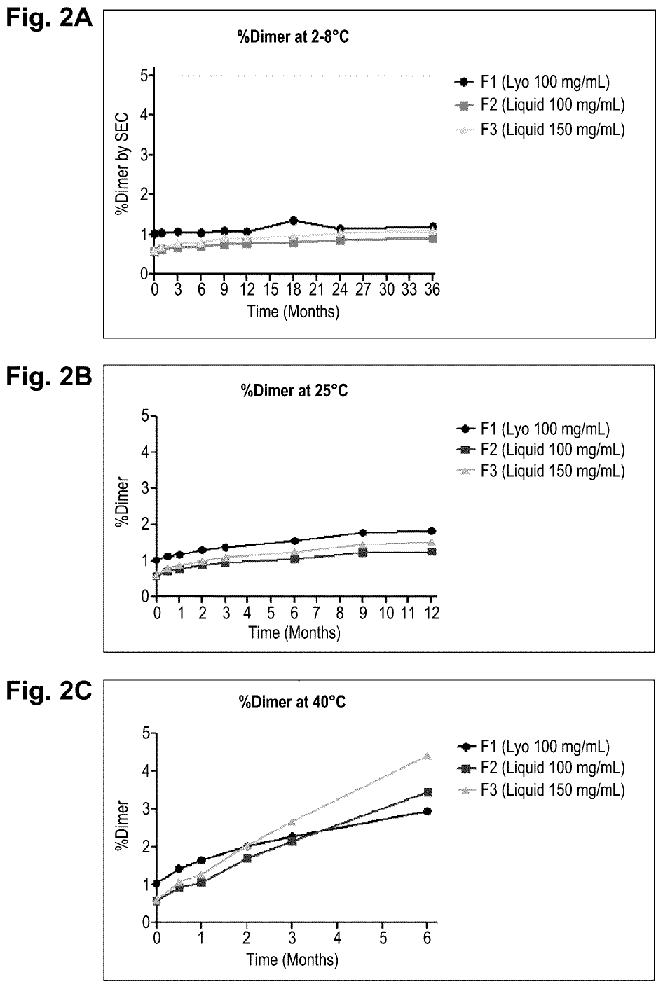

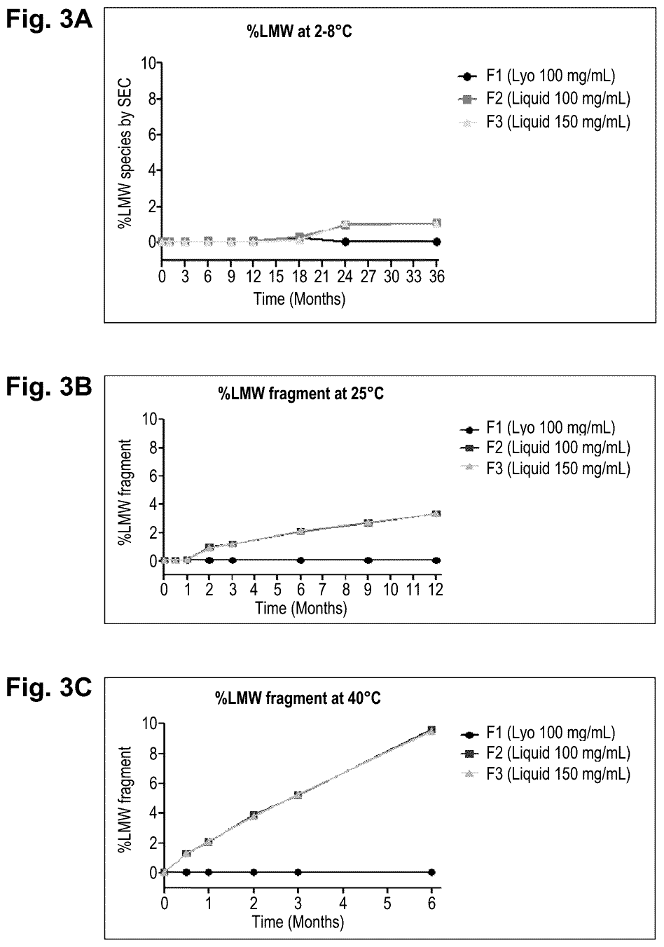

Tables 11-13 and FIGS. 1A-1C, 2A-2C and 3A-3C show the percent (%) monomer, % dimer and % low molecular weight species of Formulations 1, 2 and 3 measured by SEC. All three formulations stored at standard storage conditions of 2-8° C. met the acceptance criteria of % monomer and % dimer levels for up to 24 months and for up to 36 months. A slight decrease in % monomer with concurrent increase in % low molecular weight species was observed for Formulations 2 and 3 compared to Formulation 1. However, this is an expected observation considering that lyophilized formulations are typically more stable compared to liquid formulations. At accelerated and stressed conditions, protein fragmentation was more prevalent, indicating formation of low molecular weight species as the primary degradation pathway for liquid formulations. The rates of protein degradation of Formulation 2 and Formulation 3 were similar, indicating no significant impact of protein concentration on degradation. No significant differences were observed between Formulation 3 filled into Nipro PFS compared to Formulation 3 stored in glass vials with respect to 00 monomer, 00 dimer, and 00 low molecular weight species up to 24 months (data not shown), indicating that the stability profile of the formulation is not affected by contact with Nipro PFS.

TABLE 11
% Monomer, % Dimer and % Low Molecular Weight Species at 2-8° C.

| | Formulation 1 | | | Formulation 2 | |
|---|---|---|---|---|---|
| | % Monomer | % Dimer | | % Monomer | % Dimer |
| Time | (Acceptance | (Acceptance | % | (Acceptance | (Acceptance |
| (Months) | criteria: ≥95.0%) | criteria: ≤5.0%) | LMW | criteria: ≥95.0%) | criteria: ≤5.0%) |
| 0 | 98.93 | 1.03 | 0.04 | 99.39 | 0.57 |
| 1 | 98.92 | 1.04 | 0.04 | 99.34 | 0.62 |
| 3 | 98.90 | 1.06 | 0.04 | 99.26 | 0.68 |
| 6 | 98.90 | 1.05 | 0.05 | 99.20 | 0.70 |
| 9 | 98.85 | 1.09 | 0.04 | 99.16 | 0.77 |
| 12 | 98.86 | 1.08 | 0.03 | 99.12 | 0.78 |
| 18 | 98.56 | 1.35 | 0.29 | 98.84 | 0.80 |
| 24 | 98.70 | 1.14 | 0.04 | 98.07 | 0.85 |
| 36 | 98.70 | 1.19 | 0.04 | 98.00 | 0.91 |

| | | Formulation 3 | | |
|---|---|---|---|---|
| | Formulation 2 | % Monomer | % Dimer | |
| Time | % | (Acceptance | (Acceptance | % |
| (Months) | LMW | criteria: ≥95.0%) | criteria: ≤5.0%) | LMW |
| 0 | 0.04 | 99.35 | 0.60 | 0.04 |
| 1 | 0.04 | 99.27 | 0.68 | 0.05 |
| 3 | 0.05 | 99.17 | 0.78 | 0.06 |
| 6 | 0.09 | 99.11 | 0.81 | 0.08 |
| 9 | 0.07 | 99.02 | 0.91 | 0.07 |
| 12 | 0.09 | 98.98 | 0.92 | 0.08 |
| 18 | 0.33 | 98.84 | 0.97 | 0.16 |
| 24 | 1.00 | 97.80 | 1.04 | 1.11 |
| 36 | 1.10 | 97.80 | 1.09 | 1.10 |

LMW: Low molecular weight species

TABLE 12
% Monomer, % Dimer and % Low Molecular Weight Species at 25° C.

| | Formulation 1 | | | Formulation 2 | | | Formulation 3 | | |
|---|---|---|---|---|---|---|---|---|---|
| Time | % | % | % | % | % | % | % | % | % |
| (Months) | Monomer | Dimer | LMW | Monomer | Dimer | LMW | Monomer | Dimer | LMW |
| 0 | 98.93 | 1.03 | 0.04 | 99.39 | 0.57 | 0.04 | 99.35 | 0.60 | 0.04 |
| 0.5 | 98.83 | 1.13 | 0.04 | 99.21 | 0.71 | 0.08 | 99.11 | 0.80 | 0.08 |
| 1 | 98.77 | 1.19 | 0.04 | 99.11 | 0.78 | 0.11 | 99.00 | 0.89 | 0.11 |
| 2 | 98.65 | 1.31 | 0.04 | 98.12 | 0.89 | 0.99 | 98.08 | 1.00 | 0.92 |
| 3 | 98.56 | 1.39 | 0.05 | 97.84 | 0.95 | 1.21 | 97.68 | 1.10 | 1.22 |
| 6 | 98.39 | 1.55 | 0.06 | 96.84 | 1.06 | 2.10 | 96.62 | 1.26 | 2.12 |
| 9 | 98.18 | 1.77 | 0.04 | 96.04 | 1.23 | 2.72 | 95.81 | 1.46 | 2.73 |
| 12 | 98.08 | 1.83 | 0.03 | 95.36 | 1.26 | 3.33 | 95.06 | 1.52 | 3.36 |

LMW: Low molecular weight species

TABLE 13
% Monomer, % Dimer and % Low Molecular Weight at 40° C.

| | Formulation 1 | | | Formulation 2 | | | Formulation 3 | | |
|---|---|---|---|---|---|---|---|---|---|
| Time | % | % | % | % | % | % | % | % | % |
| (Months) | Monomer | Dimer | LMW | Monomer | Dimer | LMW | Monomer | Dimer | LMW |
| 0 | 98.93 | 1.03 | 0.04 | 99.39 | 0.57 | 0.04 | 99.35 | 0.60 | 0.04 |
| 0.5 | 98.54 | 1.42 | 0.04 | 97.75 | 0.92 | 1.33 | 97.59 | 1.07 | 1.34 |
| 1 | 98.30 | 1.65 | 0.04 | 96.87 | 1.05 | 2.08 | 96.58 | 1.28 | 2.13 |
| 2 | 97.92 | 2.04 | 0.04 | 94.41 | 1.70 | 3.89 | 94.13 | 2.06 | 3.80 |
| 3 | 97.67 | 2.29 | 0.04 | 92.61 | 2.16 | 5.23 | 92.06 | 2.68 | 5.26 |
| 6 | 96.99 | 2.95 | 0.06 | 86.36 | 3.45 | 9.60 | 85.26 | 4.42 | 9.54 |

LMW: Low molecular weight species

### Capillary Gel Electrophoresis (Reducing and Non-Reducing)

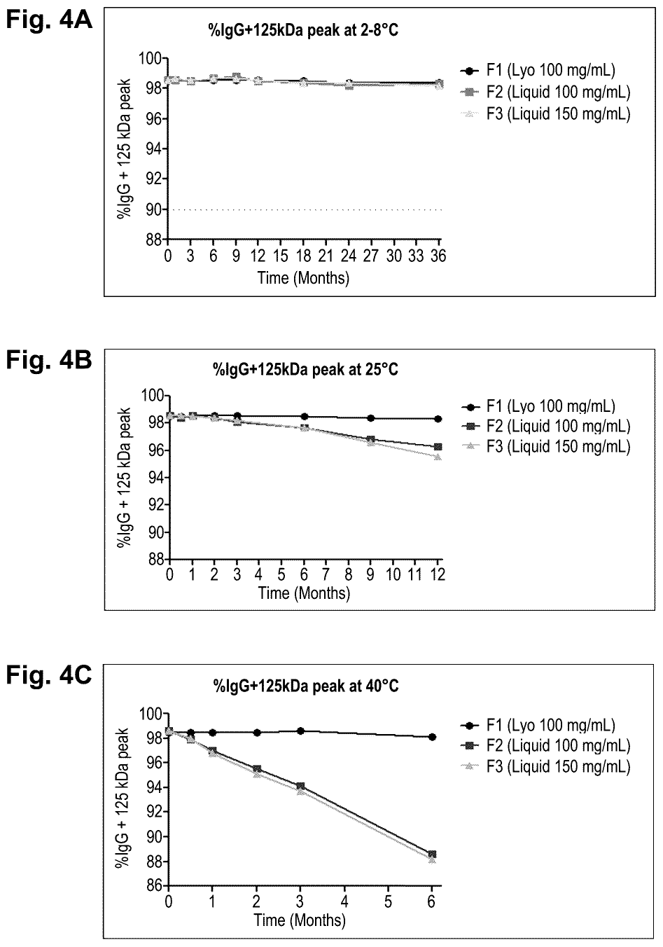

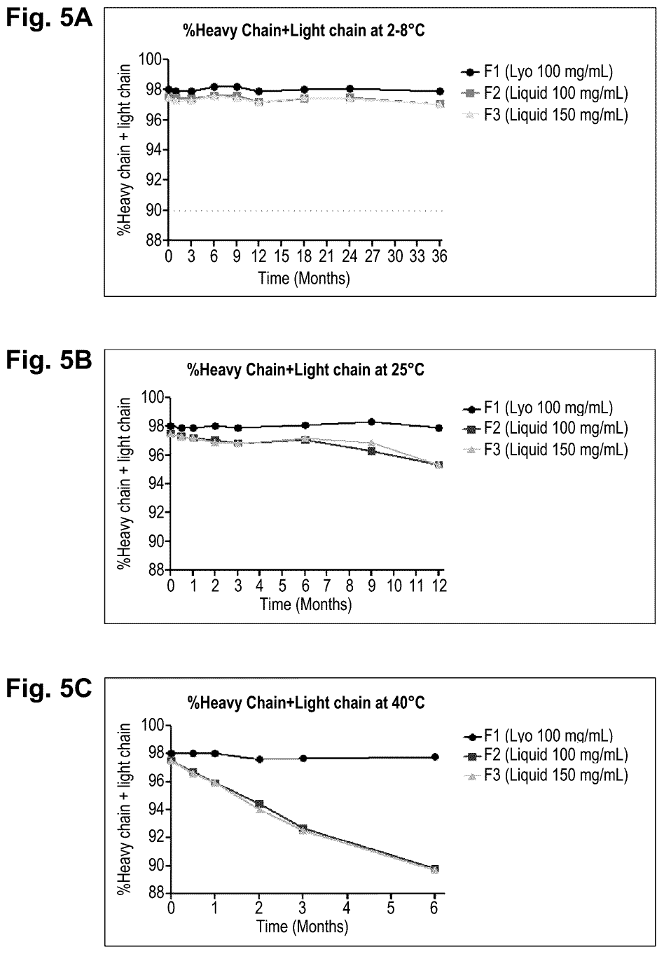

The percent (%) immunoglobulin G (IgG)+125 kDa peak was determined for Formulations 1-3 using non-reducing capillary gel electrophoresis (CGE). In addition, the % heavy chain+light chain was determined for Formulations 1-3 using reducing CGE. These results are shown in Tables 14-15 and FIGS. 4A-4C and 5A-5C, respectively. In all three formulations stored at standard storage conditions of 2-8° C., the % purity met acceptance criteria for up to 24 months and for up to 36 months. At accelerated and stressed conditions, protein fragmentation was observed for Formulations 2 and 3 compared to Formulation 1. However, this is an expected observation considering that lyophilized formulations are typically more stable compared to liquid formulations. In addition, the protein degradation trends for Formulations 2 and 3 were similar, indicating no significant impact of protein concentration. No significant differences were observed between Formulation 3 filled into Nipro PFS compared to Formulation 3 stored in glass vials with respect to the % IgG+125 kDa Peak up to 24 months (data not shown), indicating that the stability profile of the formulation is not affected by contact with Nipro PFS.

TABLE 14
% IgG + 125 kDa Peak Measured Using Non-Reducing CGE for Formulations
1, 2 and 3 at 2-8° C., 25° C. and 40° C.

| | F1 | F2 | F3 | F1 | F2 | F3 | F1 | F2 | F3 |
|---|---|---|---|---|---|---|---|---|---|
| Time | 2-8° C.; Acceptance | | | 25° C. | | | 40° C. | | |
| (Months) | criteria: ≥90.0% | | | Conditions | | | Conditions | | |
| 0 | 98.5 | 98.6 | 98.6 | 98.5 | 98.6 | 98.6 | 98.5 | 98.6 | 98.6 |
| 0.5 | NT | NT | NT | 98.5 | 98.4 | 98.6 | 98.5 | 97.9 | 98.0 |
| 1 | 98.6 | 98.6 | 98.7 | 98.6 | 98.6 | 98.5 | 98.5 | 97.0 | 96.8 |
| 2 | NT | NT | NT | 98.6 | 98.4 | 98.4 | 98.5 | 95.5 | 95.1 |
| 3 | 98.5 | 98.5 | 98.6 | 98.6 | 98.1 | 98.2 | 98.6 | 94.1 | 93.7 |
| 6 | 98.6 | 98.7 | 98.7 | 98.5 | 97.7 | 97.7 | 98.1 | 88.6 | 88.2 |
| 9 | 98.6 | 98.8 | 98.7 | 98.4 | 96.8 | 96.6 | NT | NT | NT |
| 12 | 98.6 | 98.5 | 98.6 | 98.3 | 96.3 | 95.6 | NT | NT | NT |
| 18 | 98.5 | 98.4 | 98.4 | NT | NT | NT | NT | NT | NT |
| 24 | 98.4 | 98.2 | 98.4 | NT | NT | NT | NT | NT | NT |
| 36 | 98.4 | 98.3 | 98.2 | NT | NT | NT | NT | NT | NT |

NT: Test not performed

TABLE 15
% Heavy Chain and Light Chain Measured Using Reducing CGE for Formulation
1, 2 and 3 at 2-8° C., 25° C. and 40° C.

| | F1 | F2 | F3 | F1 | F2 | F3 | F1 | F2 | F3 |
|---|---|---|---|---|---|---|---|---|---|
| Time | 2-8° C.; Acceptance | | | 25° C. | | | 40° C. | | |
| (Months) | criteria: ≥90.0% | | | Conditions | | | Conditions | | |
| 0 | 98.0 | 97.5 | 97.5 | 98.0 | 97.5 | 97.5 | 98.0 | 97.5 | 97.5 |
| 0.5 | NT | NT | NT | 97.9 | 97.3 | 97.3 | 98.0 | 96.7 | 96.6 |
| 1 | 97.9 | 97.5 | 97.3 | 97.9 | 97.2 | 97.2 | 98.0 | 95.9 | 95.9 |
| 2 | NT | NT | NT | 98.0 | 97.1 | 96.9 | 97.6 | 94.4 | 94.0 |
| 3 | 97.9 | 97.4 | 97.3 | 97.9 | 96.8 | 96.8 | 97.7 | 92.7 | 92.5 |
| 6 | 98.2 | 97.6 | 97.6 | 98.1 | 97.1 | 97.2 | 97.8 | 89.8 | 89.7 |
| 9 | 98.2 | 97.6 | 97.5 | 98.3 | 96.3 | 96.9 | NT | NT | NT |
| 12 | 97.9 | 97.2 | 97.2 | 97.9 | 95.3 | 95.3 | NT | NT | NT |
| 18 | 98.0 | 97.4 | 97.5 | NT | NT | NT | NT | NT | NT |
| 24 | 98.1 | 97.5 | 97.4 | NT | NT | NT | NT | NT | NT |
| 36 | 97.9 | 97.1 | 97.1 | NT | NT | NT | NT | NT | NT |

NT: Test not performed

Capillary Isoelectric Focusing (icIEF)

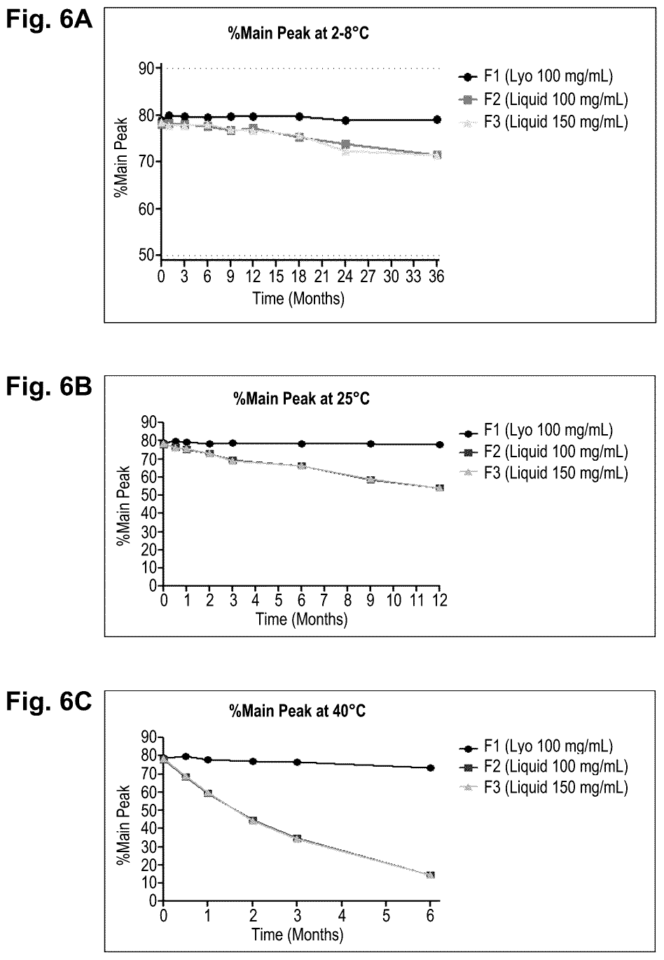

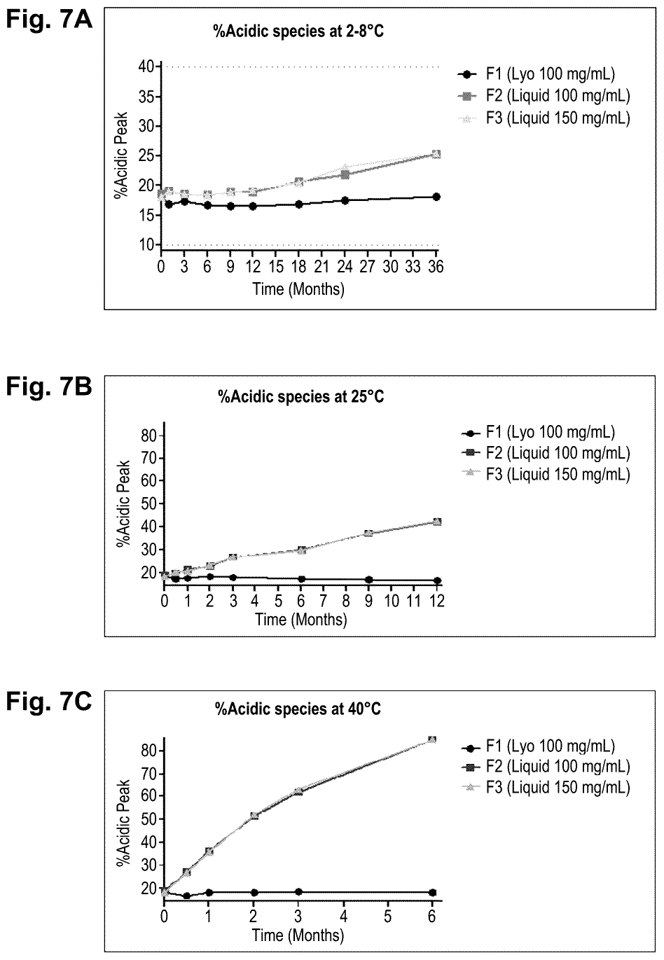

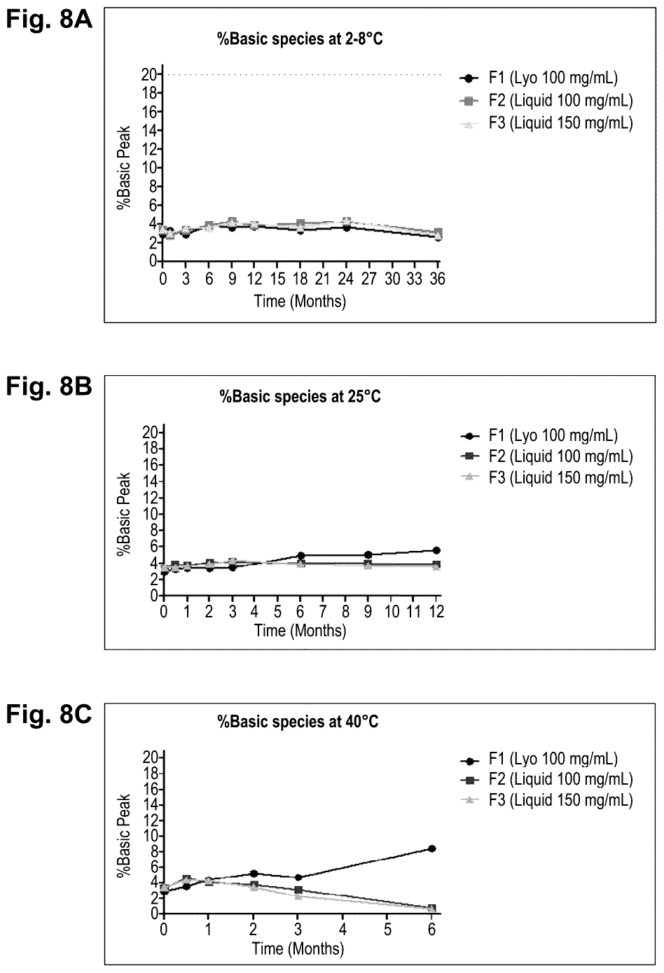

The percent (0%) content of main species (main peak), 00 acidic species (acidic peak), and 00 basic species (basic peak) of Formulations 1-3 (F1-F3) were measured using capillary isoelectric focusing (icIEF). The results are shown in Tables 16-18. An overlay of stability trends in charge heterogeneity is presented in FIGS. 6A-6C, 7A-7C and 8A-8C. At standard storage conditions of 2-8° C. (FIGS. 6A, 7A and 8A), all three formulations (F1-F3) met acceptance criteria for up to 24 months and for up to 36 months. At accelerated (25° C.; FIGS. 6B, 7B and 8B) and stressed (40° C.; FIGS. 6C, 7C and 8C) storage conditions, a decrease in 00 main peak with a concurrent increase in 00 acidic species was observed for Formulations 2 and 3. No significant change was observed for Formulation 1. However, this is an expected observation considering that lyophilized formulations are typically more stable compared to liquid formulations. No significant differences were observed between Formulation 3 filled into Nipro PFS compared to Formulation 3 stored in glass vials with respect to % main peak, % acidic species and % basic species up to 24 months (data not shown), indicating that the stability profile of the formulation is not affected by contact with Nipro PFS.

TABLE 16
% Main Peak, % Acidic Species and % Basic Species Determined
Using icIEF for Formulations 1, 2 and 3 at 2-8° C.

| | Formulation 1 at 2-8° C. | | | Formulation 2 at 2-8° C. | | | Formulation 3 at 2-8° C. | | |
|---|---|---|---|---|---|---|---|---|---|
| | % Main | % Acidic | % Basic | % Main | % Acidic | % Basic | % Main | % Acidic | % Basic |
| | peak | species | species | peak | species | species | peak | species | species |
| | (Accep- | (Accep- | (Accep- | (Accep- | (Accep- | (Accep- | (Accep- | (Accep- | (Accep- |
| | tance | tance | tance | tance | tance | tance | tance | tance | tance |
| Time | criteria: | criteria: | criteria: | criteria: | criteria: | criteria: | criteria: | criteria: | criteria: |
| (Months) | 50-90%) | 10-40%) | 0-20%) | 50-90%) | 10-40%) | 0-20%) | 50-90%) | 10-40%) | 0-20%) |
| 0.0 | 79.0 | 18.1 | 2.9 | 78.1 | 18.6 | 3.3 | 78.5 | 18.1 | 3.5 |
| 1.0 | 79.9 | 16.9 | 3.2 | 78.1 | 19.0 | 2.8 | 77.8 | 19.1 | 3.0 |
| 3.0 | 79.7 | 17.4 | 2.9 | 78.1 | 18.6 | 3.4 | 77.8 | 18.6 | 3.6 |
| 6.0 | 79.4 | 16.7 | 3.9 | 77.7 | 18.4 | 3.9 | 78.0 | 18.4 | 3.7 |
| 9.0 | 79.7 | 16.6 | 3.7 | 76.7 | 19.0 | 4.3 | 76.9 | 18.9 | 4.2 |
| 12.0 | 79.7 | 16.5 | 3.8 | 77.1 | 19.0 | 3.9 | 76.7 | 19.2 | 4.1 |
| 18.0 | 79.7 | 16.9 | 3.4 | 75.3 | 20.7 | 4.1 | 75.7 | 20.6 | 3.7 |
| 24.0 | 78.8 | 17.5 | 3.7 | 73.9 | 21.8 | 4.3 | 72.3 | 23.3 | 4.4 |
| 36.0 | 79.1 | 18.2 | 2.6 | 71.4 | 25.4 | 3.2 | 71.6 | 25.5 | 2.9 |

TABLE 17
% Main Peak, % Acidic Species and % Basic Species Determined
Using icIEF for Formulations 1, 2 and 3 at 25° C.

| | Formulation 1 at 25° C. | | | Formulation 2 at 25° C. | | | Formulation 3 at 25° C. | | |
|---|---|---|---|---|---|---|---|---|---|
| Time | % Main | % Acidic | % Basic | % Main | % Acidic | % Basic | % Main | % Acidic | % Basic |
| (Months) | peak | species | species | peak | species | species | peak | species | species |
| 0.0 | 79.0 | 18.1 | 2.9 | 78.1 | 18.6 | 3.3 | 78.5 | 18.1 | 3.5 |
| 0.5 | 79.7 | 17.0 | 3.3 | 76.8 | 19.3 | 3.9 | 76.2 | 20.3 | 3.5 |
| 1.0 | 79.2 | 17.5 | 3.3 | 75.1 | 21.1 | 3.8 | 75.7 | 20.6 | 3.7 |
| 2.0 | 78.6 | 18.0 | 3.4 | 73.1 | 22.8 | 4.1 | 73.1 | 23.0 | 3.9 |
| 3.0 | 78.9 | 17.6 | 3.5 | 69.2 | 26.6 | 4.2 | 69.0 | 26.5 | 4.5 |
| 6.0 | 78.3 | 16.8 | 4.9 | 66.1 | 29.9 | 4.0 | 66.4 | 29.7 | 3.9 |
| 9.0 | 78.4 | 16.5 | 5.1 | 58.8 | 37.2 | 4.0 | 58.9 | 37.4 | 3.7 |
| 12.0 | 78.1 | 16.3 | 5.6 | 54.1 | 42.0 | 3.9 | 53.9 | 42.5 | 3.6 |

TABLE 18
% Main Peak, % Acidic Species and % Basic Species Determined
Using icIEF for Formulations 1, 2 and 3 at 40° C.

| | Formulation 1 at 40° C. | | | Formulation 2 at 40° C. | | | Formulation 3 at 40° C. | | |
|---|---|---|---|---|---|---|---|---|---|
| Time | % Main | % Acidic | % Basic | % Main | % Acidic | % Basic | % Main | % Acidic | % Basic |
| (Months) | peak | species | species | peak | species | species | peak | species | species |
| 0.0 | 79.0 | 18.1 | 2.9 | 78.1 | 18.6 | 3.3 | 78.5 | 18.1 | 3.5 |
| 0.5 | 79.7 | 16.6 | 3.6 | 68.4 | 27.0 | 4.7 | 68.9 | 26.8 | 4.4 |
| 1.0 | 77.7 | 17.9 | 4.4 | 59.5 | 36.4 | 4.1 | 60.0 | 35.8 | 4.3 |
| 2.0 | 76.9 | 17.9 | 5.2 | 44.6 | 51.6 | 3.8 | 44.3 | 52.2 | 3.5 |
| 3.0 | 76.8 | 18.5 | 4.7 | 34.9 | 61.9 | 3.2 | 34.3 | 63.4 | 2.3 |
| 6.0 | 73.6 | 18.0 | 8.4 | 14.3 | 84.8 | 0.9 | 14.3 | 84.9 | 0.8 |

### Sub-Visible Particles Using Micro-Flow Imaging (MFI)

Sub-visible particles in Formulations 1-3 were measured at different time points at standard conditions of 2-8° C., accelerated conditions of 25° C., and stressed conditions of 40° C. The results are shown in Tables 19-21, respectively. No significant changes in sub-visible particles were observed. At intermittent time points, higher sub-visible particles were observed compared to other time points. This could be related to the method where greater variability and sensitivity have been observed for sub-visible particles measured using MFI. Overall, sub-visible particles in the size range of ≥(more than or equal to) 10 m were less than 6,000 particles/mL. In the size range of ≥ (more than or equal to) 25 m, the sub-visible particles were less than 600 particles/mL and were well within USP<788> limits even considering the increased sensitivity of using MFI for sub-visible particle detection. No significant differences were observed between Formulation 3 filled into Nipro PFS compared to Formulation 3 stored in glass vials with respect to sub-visible particles up to 24 months (data not shown), indicating that the stability profile of the formulation is not affected by contact with Nipro PFS.

TABLE 19
Sub-Visible Particles for Formulations 1-3 at 2-8° C.

| | SbVPs ≥2 μm | | | SbVPs ≥10 μm | | | SbVPs ≥25 μm | | |
|---|---|---|---|---|---|---|---|---|---|
| | (particles/mL) | | | (particles/mL) | | | (particles/mL) | | |
| Time | at 2-8° C. | | | at 2-8° C. | | | at 2-8° C. | | |
| (Months) | F1 | F2 | F3 | F1 | F2 | F3 | F1 | F2 | F3 |
| 0 | 39058 | 844 | 612 | 175 | 141 | 25 | 10 | 8 | 2 |
| 1 | 34155 | 1008 | 1790 | 393 | 98 | 59 | 7 | 20 | 0 |
| 3 | 35353 | 1494 | 1521 | 225 | 52 | 19 | 3 | 0 | 3 |
| 6 | 38899 | 2398 | 10228 | 160 | 143 | 676 | 0 | 30 | 59 |
| 9 | 50016 | 3378 | 4969 | 533 | 204 | 251 | 25 | 32 | 37 |
| 12 | 54427 | 2452 | 4158 | 376 | 59 | 98 | 10 | 15 | 5 |
| 18 | 29704 | 1463 | 1330 | 184 | 68 | 55 | 17 | 9 | 6 |
| 24 | 20591 | 9289 | 11269 | 297 | 406 | 398 | 26 | 22 | 11 |
| 36 | 18716 | 2534 | 7578 | 354 | 212 | 297 | 107 | 48 | 39 |

SbVPs: Sub-visible particles

TABLE 20
Sub-Visible Particles for Formulations 1-3 at 25° C.

| | Sb VPs ≥ 2 μm | | | SbVPs ≥ 10 μm | | | SbVPs ≥ 25 μm | | |
|---|---|---|---|---|---|---|---|---|---|
| Time | (particles/mL) at 25° C. | | | (particles/mL) at 25° C. | | | (particles/mL) at 25° C. | | |
| (Months) | F1 | F2 | F3 | F1 | F2 | F3 | F1 | F2 | F3 |
| 0 | 39058 | 844 | 612 | 175 | 141 | 25 | 10 | 8 | 2 |
| 0.5 | 38785 | 1411 | 1086 | 216 | 120 | 89 | 7 | 12 | 22 |
| 1 | 26524 | 937 | 1369 | 246 | 69 | 30 | 17 | 10 | 5 |
| 2 | 34338 | 6140 | 2159 | 130 | 173 | 65 | 9 | 6 | 19 |
| 3 | 39657 | 1022 | 6339 | 114 | 37 | 96 | 6 | 6 | 12 |
| 6 | 15358 | 13316 | 14585 | 79 | 858 | 910 | 15 | 69 | 89 |
| 9 | 27019 | 4556 | 10106 | 209 | 240 | 683 | 32 | 21 | 61 |
| 12 | 62324 | 2256 | 10055 | 631 | 302 | 2061 | 7 | 37 | 268 |

SbVPs: Sub-visible particles

TABLE 21
Sub-Visible Particles for Formulations 1-3 at 40° C.

| | SbVPs ≥ 2 μm | | | SbVPs ≥ 10 μm | | | SbVPs ≥ 25 μm (particles/mL) | | |
|---|---|---|---|---|---|---|---|---|---|
| Time | (particles/mL) at 25° C. | | | (particles/mL) at 25° C. | | | at 25° C. | | |
| (Months) | F1 | F2 | F3 | F1 | F2 | F3 | F1 | F2 | F3 |
| 0 | 39058 | 844 | 612 | 175 | 141 | 25 | 10 | 8 | 2 |
| 0.5 | 67190 | 740 | 2311 | 268 | 32 | 197 | 12 | 5 | 39 |
| 1 | 59549 | 1428 | 4399 | 511 | 32 | 241 | 5 | 5 | 15 |
| 2 | 42669 | 5810 | 7054 | 275 | 272 | 457 | 3 | 25 | 25 |
| 3 | 40732 | 1974 | 7686 | 170 | 86 | 488 | 9 | 0 | 25 |
| 6 | 29017 | 12221 | 12188 | 216 | 681 | 755 | 32 | 34 | 106 |

### Chemical Modifications to Primary Structure Using Peptide Mapping

Amino acids that could potentially undergo chemical modification over time and influence protein structure were monitored for Formulations 1-3. Specifically, amino acid residues methionine 81 and methionine 254 of TEV-48574 could potentially undergo oxidation, affecting the primary structure, and were monitored. Similarly, asparagine 317 of TEV-48574 could potentially undergo deamidation, resulting in succinimide, and were monitored. However, as shown in Tables 22-24, no significant changes were observed in these modifications over time. Results were comparable between Formulation 3 filled into Nipro PFS compared to Formulation 3 stored in glass vials at the long-term storage condition (2-8° C.) up to 24 months with respect to % Met81 oxidation, 00 Asn317 deamidation and 0% Met254 oxidation (data not shown), indicating that the stability profile of the formulation is not affected by contact with Nipro PFS.

TABLE 22
% Met81 Oxidation in Formulations 1-3

| | % Met81 | | | % Met81 | | | % Met81 | | |
|---|---|---|---|---|---|---|---|---|---|
| | Oxidation | | | Oxidation | | | Oxidation | | |
| Time | at 2-8° C. | | | at 25° C. | | | at 40° C. | | |
| (Months) | F1 | F2 | F3 | F1 | F2 | F3 | F1 | F2 | F3 |
| 0 | 2.1 | 1.1 | 0.5 | 2.1 | 1.1 | 0.5 | 2.1 | 1.1 | 0.5 |
| 1 | 3.1 | 1.2 | 2.2 | 1.2 | 0.6 | 1.0 | 4.9 | 1.3 | 1.1 |
| 3 | 1.2 | 2.3 | 0.8 | 0.8 | 1.5 | 0.8 | 0.9 | 1.7 | 1.0 |
| 6 | 0.6 | 0.6 | 0.7 | 0.5 | 0.6 | 0.6 | 0.7 | 0.6 | 0.7 |
| 12 | 0.6 | 0.7 | 1.5 | 0.8 | 0.5 | 0.6 | NT | NT | NT |
| 18 | 0.8 | 0.5 | 0.6 | NT | NT | NT | NT | NT | NT |
| 24 | 1.0 | 0.9 | 1.0 | NT | NT | NT | NT | NT | NT |
| 36 | 0.3 | 0.3 | 0.4 | NT | NT | NT | NT | NT | NT |

NT: Test not performed;
F1: Formulation 1;
F2: Formulation 2;
F3: Formulation 3;
Met81: methionine residue 81 of TEV-48574

TABLE 23
% Asn317 Deamidation in Formulations 1-3

| | % Asn317 | | | % Asn317 | | | % Asn317 | | |
|---|---|---|---|---|---|---|---|---|---|
| | deamidation | | | deamidation | | | deamidation | | |
| Time | at 2-8° C. | | | at 25° C. | | | at 40° C. | | |
| (Months) | F1 | F2 | F3 | F1 | F2 | F3 | F1 | F2 | F3 |
| 0 | 7.8 | 7.4 | 7.8 | 7.8 | 7.4 | 7.8 | 7.8 | 7.4 | 7.8 |
| 1 | 8.6 | 7.7 | 8.7 | 7.6 | 7.4 | 7.5 | 8.0 | 7.6 | 8.3 |
| 3 | 7.1 | 7.9 | 7.4 | 8.0 | 8.1 | 6.6 | 7.9 | 7.7 | 7.2 |
| 6 | 8.4 | 7.0 | 6.8 | 7.2 | 6.0 | 5.0 | 7.0 | 7.3 | 8.7 |
| 12 | 9.6 | 9.8 | 9.1 | 8.9 | 9.7 | 10.0 | NT | NT | NT |
| 18 | 7.6 | 5.4 | 6.5 | NT | NT | NT | NT | NT | NT |
| 24 | 9.3 | 10.0 | 11.2 | NT | NT | NT | NT | NT | NT |
| 36 | 7.1 | 7.8 | 10.0 | NT | NT | NT | NT | NT | NT |

NT: Test not performed;
F1: Formulation 1;
F2: Formulation 2;
F3: Formulation 3;
Asn317: asparagine residue 317 of TEV-48574

TABLE 24
% Met254 Oxidation in Formulations 1-3

| | % Met254 | | | % Met254 | | | % Met254 | | |
|---|---|---|---|---|---|---|---|---|---|
| | Oxidation | | | Oxidation | | | Oxidation | | |
| Time | at 2-8° C. | | | at 25° C. | | | at 40° C. | | |
| (Months) | F1 | F2 | F3 | F1 | F2 | F3 | F1 | F2 | F3 |
| 0 | 3.7 | 2.4 | 1.5 | 3.7 | 2.4 | 1.5 | 3.7 | 2.4 | 1.5 |
| 1 | 4.9 | 2.5 | 3.9 | 2.3 | 2.2 | 2.5 | 7.4 | 4.4 | 3.9 |
| 3 | 2.5 | 4.3 | 2.3 | 1.9 | 4.2 | 3.1 | 1.9 | 9.5 | 8.7 |
| 6 | 1.8 | 2.3 | 2.4 | 1.8 | 4.3 | 4.5 | 1.8 | 18.8 | 20.5 |
| 12 | 1.7 | 2.6 | 3.9 | 1.9 | 6.4 | 5.6 | NT | NT | NT |
| 18 | 1.8 | 2.8 | 3.3 | NT | NT | NT | NT | NT | NT |
| 24 | 2.2 | 3.9 | 4.2 | NT | NT | NT | NT | NT | NT |
| 36 | 1.6 | 4.2 | 4.3 | NT | NT | NT | NT | NT | NT |

NT: Test not performed;
F1: Formulation 1;
F2: Formulation 2;
F3: Formulation 3;
Met254: methionine residue 254 of TEV-48574

### Potency by ELISA

The percent (0%) potency of Formulations 1-3 was determined by enzyme-linked immunosorbent assay (ELISA). The results are shown in Table 25. No significant changes were observed between the formulations at standard storage conditions (2-8° C.), and the acceptance criteria of 700-135% potency was met for up to 24 months and for up to 36 months. No significant difference was observed between Formulation 3 filled into Nipro PFS compared to Formulation 3 stored in glass vials with respect to 00 potency up to 24 months (data not shown), indicating that the stability profile of the formulation is not affected by contact with Nipro PFS.

TABLE 25
% Relative Potency of Formulations 1-3 by ELISA

| | % Potency | | | | | | | | |
|---|---|---|---|---|---|---|---|---|---|
| | at 2-8° C. | | | | | | | | |
| | Acceptance | | | % Potency | | | % Potency | | |
| Time | criteria: 70-135% | | | at 25° C. | | | at 40° C. | | |
| (Months) | F1 | F2 | F3 | F1 | F2 | F3 | F1 | F2 | F3 |
| 0 | 87 | 100 | 100 | 87 | 100 | 100 | 87 | 100 | 100 |
| 0.5 | NT | NT | NT | 92 | 100 | 90 | 95 | 95 | 94 |
| 1 | 101 | 97 | 98 | 99 | 90 | 96 | 104 | 103 | 98 |
| 2 | NT | NT | NT | 96 | 92 | 91 | 104 | 100 | 90 |
| 3 | 98 | 92 | 88 | 92 | 95 | 92 | 110 | 98 | 89 |
| 6 | 92 | 88 | 87 | 97 | 95 | 96 | 92 | 84 | 86 |
| 9 | 101 | 93 | 95 | 99 | 92 | 90 | NT | NT | NT |
| 12 | 89 | 95 | 106 | 108 | 103 | 105 | NT | NT | NT |
| 18 | 108 | 100 | 103 | NT | NT | NT | NT | NT | NT |
| 24 | 103 | 97 | 101 | NT | NT | NT | NT | NT | NT |
| 36 | 102 | 96 | 96 | NT | NT | NT | NT | NT | NT |

NT: Test not performed

### Differential Scanning Calorimetry (DSC) Analysis for Thermal Stability

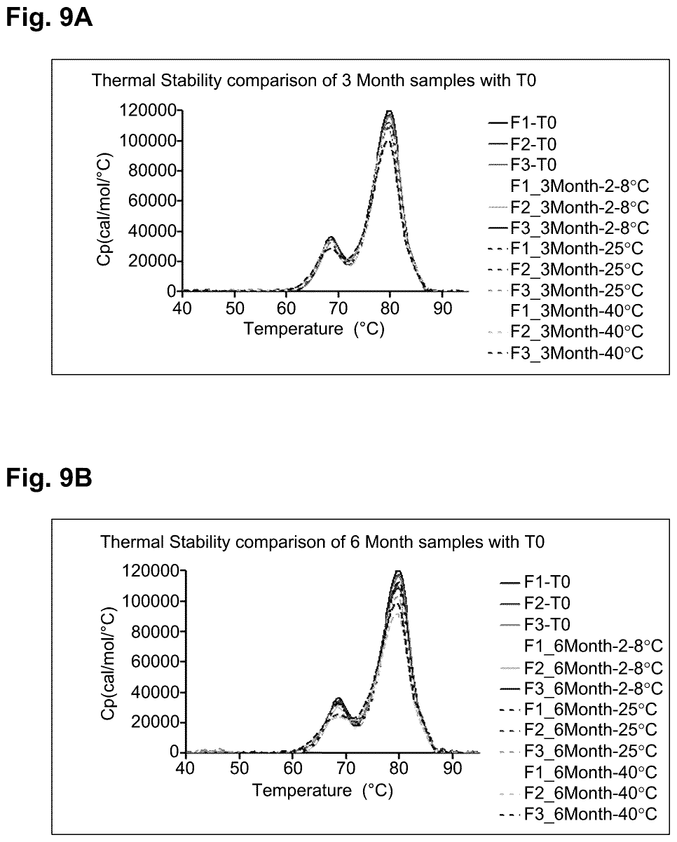

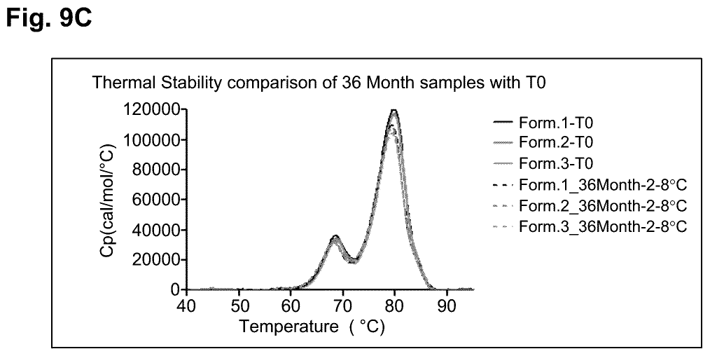

DSC was employed to evaluate thermal stability of Formulations 1-3. DSC analysis was performed for a time zero (T0) sample of Formulations 1-3 and for 3 month, 6 month, and 36 month samples at 2-8° C., 25° C. and 40° C. conditions. FIG. 9A (FIG. 9A) shows the thermal stability of Formulations 1-3 at time zero (T0) and at 2-8° C., 25° C., and 40° C. after 3 months. FIG. 9B (FIG. 9B) shows the thermal stability of Formulations 1-3 at time zero (T0) and at 2-8° C., 25° C., and 40° C. after 6 months. FIG. 9C (FIG. 9C) shows the thermal stability of Formulations 1-3 at time zero (T0) and at 2-8° C. after 36 months. No significant difference in thermal stability was observed for the three formulations at T0, 3 months, 6 months, and 36 months, as the transition temperatures (Tm) overlapped for all formulations. A small decrease in enthalpy was observed for formulations stored at accelerated and stressed conditions. This could be due to protein fragmentation observed in the samples at these conditions, leading to a decrease in enthalpy. No significant differences were observed between Formulation 3 filled into Nipro PFS compared to Formulation 3 stored in glass vials up to 6 months with respect to thermal stability, indicating that the stability profile of the formulation is not affected by contact with Nipro PFS.

### Secondary and Tertiary Protein Structure Analysis Using Circular Dichroism (CD) Spectroscopy

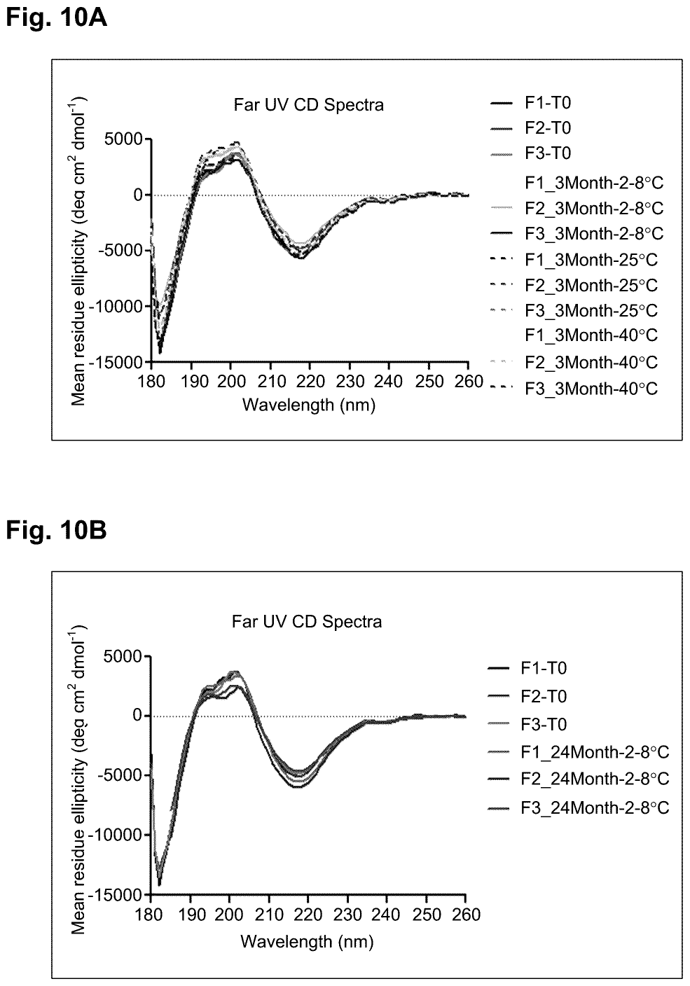

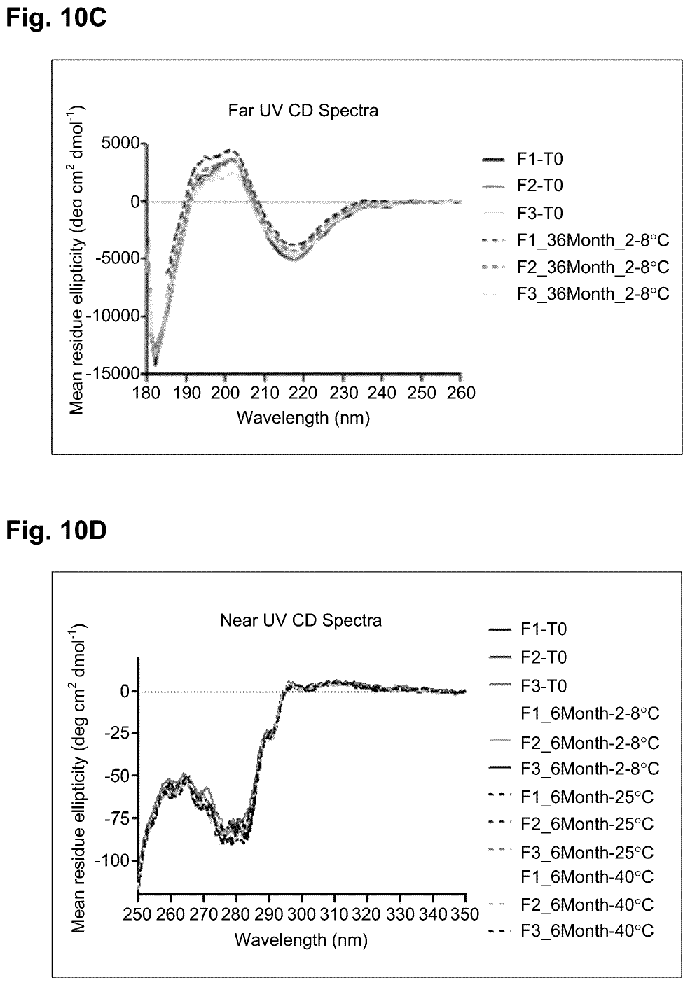

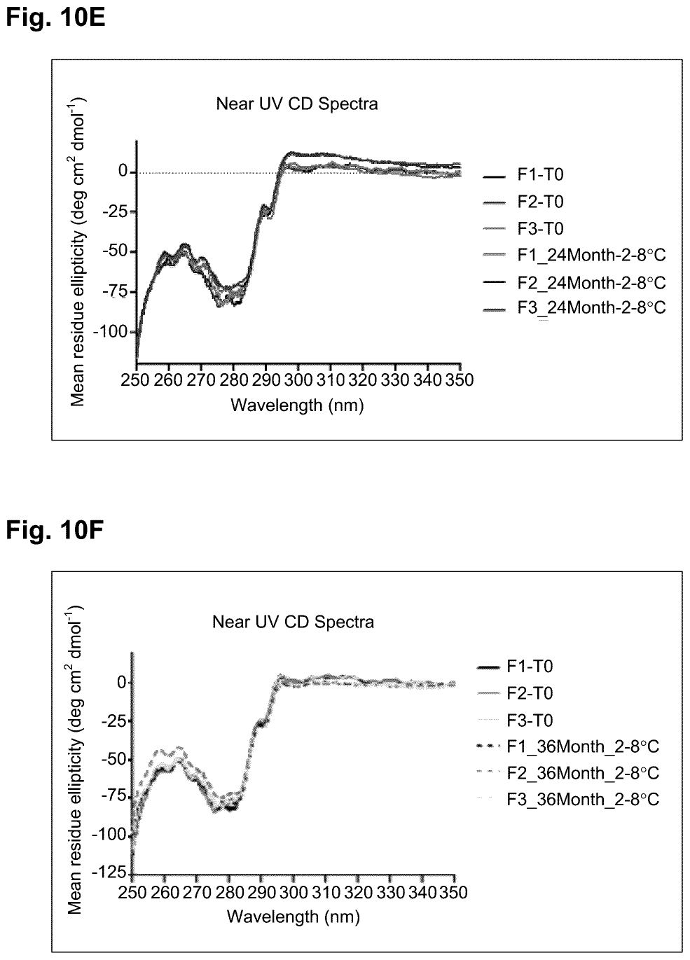

Secondary protein structure of Formulations 1-3 was analyzed using far-ultraviolet (far-UV) circular dichroism (CD), and tertiary protein structure of Formulations 1-3 was analyzed using near-UV CD. Secondary and tertiary structure analysis was performed for time zero (T0) samples of Formulations 1, 2 and 3; 3 month samples at 2-8° C., 25° C. and 40° C. conditions; 24 month samples at 2-8° C.; and 36 month samples at 2-8° C. The results are shown in FIGS. 10A-10F (FIGS. 10A-10F). Specifically, FIG. 10A shows the secondary structure of Formulations 1-3 at TO and after 3 months at 2-8° C., 25° C. and 40° C. using far UV CD. FIG. 10B shows the secondary structure of Formulations 1-3 at TO and after 24 months at 2-8° C. using far UV CD. FIG. 10C shows the secondary structure of Formulations 1-3 at TO and after 36 months at 2-8° C. using far UV CD. FIG. 10D shows the tertiary structure of Formulations 1-3 at TO and after 3 months at 2-8° C., 25° C. and 40° C. using near UV CD. FIG. 10E shows the tertiary structure of Formulations 1-3 at TO) and after 24 months at 2-8° C. using near UV CD. And, FIG. 10F shows the tertiary structure of Formulations 1-3 at TO) and after 36 months at 2-8° C. using near UV CD.

The far UV CD spectra showed negative maxima at around 217 nm, indicating beta sheet structure for T0 samples in all three formulations, which is expected for a monoclonal antibody. The near UV CD spectra showed positive maxima at around 292 nm, indicative of absorption by tryptophan residues and negative maxima at 276 nm, indicative of absorption by Tyrosine residues. No significant change in secondary structure or tertiary structure was observed for the three formulations up to 3 months. At 24 months and 36 months, small changes in CD spectra were observed at around 200 nm. This could be due to protein fragmentation since the absorption in this region is predominantly due to peptide bond absorption. However, no significant change in secondary structure of protein (absorption at 217 nm) was observed.

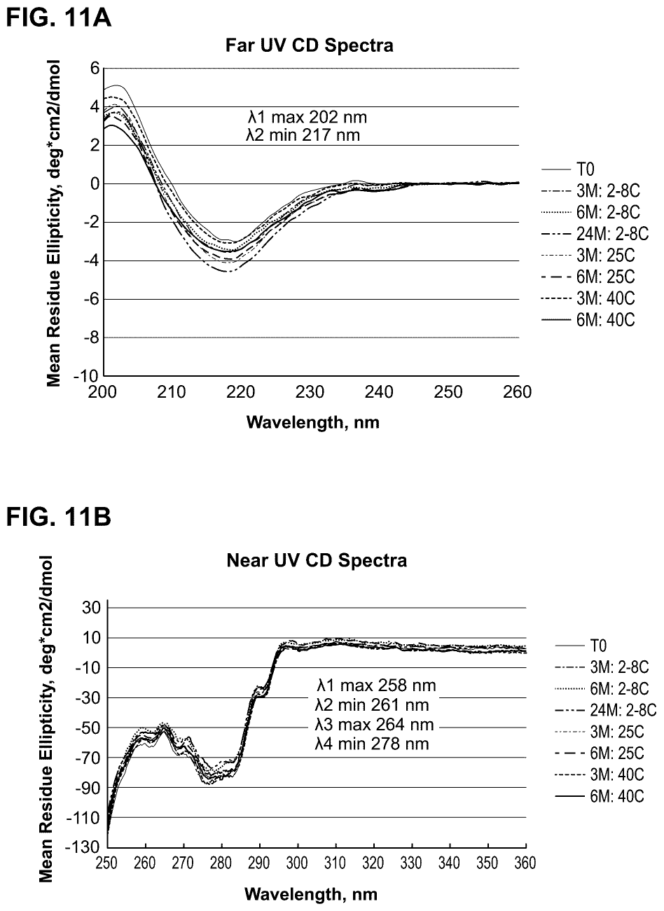

FIG. 11A shows the secondary structure of Formulation 3 stored in Nipro prefilled syringes (PFS) at time zero (T0) and after 3 months (3M), 6 months (6M), and 24 months (24M) of storage at 2-8° C., and after 3 months (3M) and 6 months (6M) of storage at 25° C. and 40° C. using far UV CD. FIG. 11B shows the tertiaty structure of Formulation 3 stored in Nipro prefilled syringes (PFS) at time zero (T0) and after 3 months (3M), 6 months (6M), and 24 months (24M) of storage at 2-8° C., and after 3 months (3M) and 6 months (6M) of storage at 25° C. and 40° C. using near UV CD. The spectra obtained for the Nipro PFS samples are comparable to those obtained for Formulation 3 stored in glass vials (FIGS. 10A-10B and 10D-10E).

### Polysorbate 80 Analysis

Polysorbate 80 (PS80) as a surfactant/excipient present in Formulations 1-3 could potentially undergo degradation, which results in reactive peroxides that over the shelf life of the product may impact protein stability. PS80 analysis was performed on Formulations 1-3 after storage for 24 months and 36 months at 2-8° C. by testing PS80 levels. As shown in Table 26, the measured PS80 levels were 0.02% (w/v) for 24 month samples in all formulations, correlating with the PS80 concentration expected. At 36 months, PS80 level in formulations 2 and 3, which are liquid formulation, showed a decrease. No significant differences were observed between Formulation 3 filled into Nipro PFS compared to Formulation 3 stored in glass vials with respect to PS80 levels at 24 months, indicating that the stability profile of the formulation is not affected by contact with Nipro PFS.

TABLE 26
Detected Polysorbate 80 (PS80) Levels in Formulations 1-3
After Storage for 24 Months and 36 Months at 2-8° C.

| | PS80 levels (w/v) | PS80 levels (w/v) |
|---|---|---|
| Formulation at 2-8° C. | at 24 M | at 36 M |
| Formulation 1 | 0.02% | 0.02% |
| Formulation 2 | 0.02% | 0.01% |
| Formulation 3 | 0.02% | 0.01% |

### Conclusions

The stability of TEV-48574 in lyophilized form (Formulation 1 at 100 mg/mL) was comparable to liquid formulations at 100 mg/mL (Formulation 2) and 150 mg/mL (Formulation 3) stored at 2-8° C., the standard storage condition for the drug product. At accelerated (25° C.) and stressed (40° C.) conditions, both liquid formulations showed protein fragmentation as the primary degradation pathway, which was not observed for lyophilized Formulation 1. However, this is an expected observation considering that lyophilized formulations are more stable compared to liquid formulations.

Formation of acidic species was observed in liquid formulations compared to the lyophilized formulation, measured using icIEF, and was concomitant with protein fragmentation observed using SEC and CGE. This indicates deamidation of certain amino acid residues, potentially causing fragmentation. See Wang, W. et al., J. Pharm. Sci., 96:1-26 (2007).

Based on these data, lyophilized Formulation 1 can be bridged to liquid formulations at 100 mg/mL and 150 mg/mL, having a comparable stability profile and meeting acceptance criteria for up to 24 months and up to 36 months at 2-8° C. storage conditions.

Furthermore, the stability of TEV-48574 at 150 mg/mL (Formulation 3) stored in Nipro PFS was found to be comparable as Formulation 3 samples stored in glass vials. Based on this data, 24 month storage at 2-8° C., the standard storage condition for the drug product, is compatible with the Nipro PFS.

### Example 3: Stability of TEV-48574 Liquid Formulation at 200 mg/ml

The purpose of this study was to evaluate the long term stability of a high concentration liquid TEV-48574 formulation in comparison to a lyophilized drug product. Two formulations were tested: a lyophilized form containing 100 mg/mL of drug product in 10 mM Histidine, 5% (w/v) Sucrose, 100 mM Arg-HCl, 0.02% (w/v) PS-80 at pH 6.0 (Formulation 4 or F4), and a liquid form containing 200 mg/mL of drug product in 10 mM Histidine, 5% (w/v) Sucrose, 100 mM Arg-HCl, 0.02% (w/v) PS-80 at pH 6.0 (Formulation 5 or F5). The stability of these formulations was evaluated in 5 cc vials with a 20 mm size stopper at the long-term storage condition of 2-8° C. The impact of these conditions on several product quality attributes was tested for the formulations.

### Visual Appearance, Protein Concentration, Osmolality and Viscosity

The results of a visual appearance, protein concentration, and osmolality analysis for Formulations 4 and 5 are shown in Tables 27-28. At the long-term storage condition of 2-8° C., no significant difference across the formulations was observed.

The measured protein concentration for both formulations at the long-term storage condition of 2-8° C. were close to the nominal concentration expected and did not vary over time. Osmolality and viscosity of the drug products were evaluated at time zero (T0). The osmolality and viscosity of the 200 mg/mL liquid formulation (F5) were observed to be higher than those observed for the lyophilized formulation (F4) at 100 mg/mL.

TABLE 27
Visual Appearance and Protein Concentration Results at 2-8° C.

| Time | | Visual appearance | | Protein concentration (mg/mL) | |
|---|---|---|---|---|---|
| Point | | F4 | F5 | F4 | F5 |
| T0 | | L; C; FFVP | SP; O; FFVP | 95.8 | 201.4 |
| 1 | M | NT | NT | 94.6 | 198.8 |
| 3 | M | L; S; FFVP | L; C; FFVP | NT | NT |
| 6 | M | L; S; FFVP | L; S; FFVP | 95.6 | 200.4 |
| 9 | M | L; V; FFVP | VSY; S; FFVP | 97.6 | 194.3 |
| 12 | M | L; C; FFVP | L; C; FFVP | 97.1 | 197.9 |
| 18 | M | L; C; FFVP | L; C; FFVP | 96.8 | 199.6 |
| 24 | M | L; C; FFVP | L; C; FFVP | 96.5 | 201.0 |

T0: Time zero;
M: Month(s);
F4: Formulation 4;
F5: Formulation 5;
S: Slightly opalescent;
O: Opaslescent;
V: Very opaslescent;
L: Colorless;
FFVP: Free from visible particles;
C: Clear;
VSY: Very slightly yellow;
SP: Slightly pink;
NT: Not tested

TABLE 28
Osmolality and Viscosity Results at 2-8° C.

| Time | Osmolality (mOsm/kg) | | Viscosity (cP) measured at 20° C. | |
|---|---|---|---|---|
| Point | F4 | F5 | F4 | F5 |
| T0 | 382 | 542 | 3 | 19 |

T0: Time zero;
F4: Formulation 4;
F5: Formulation 5

### Size Exclusion Chromatography (SEC)

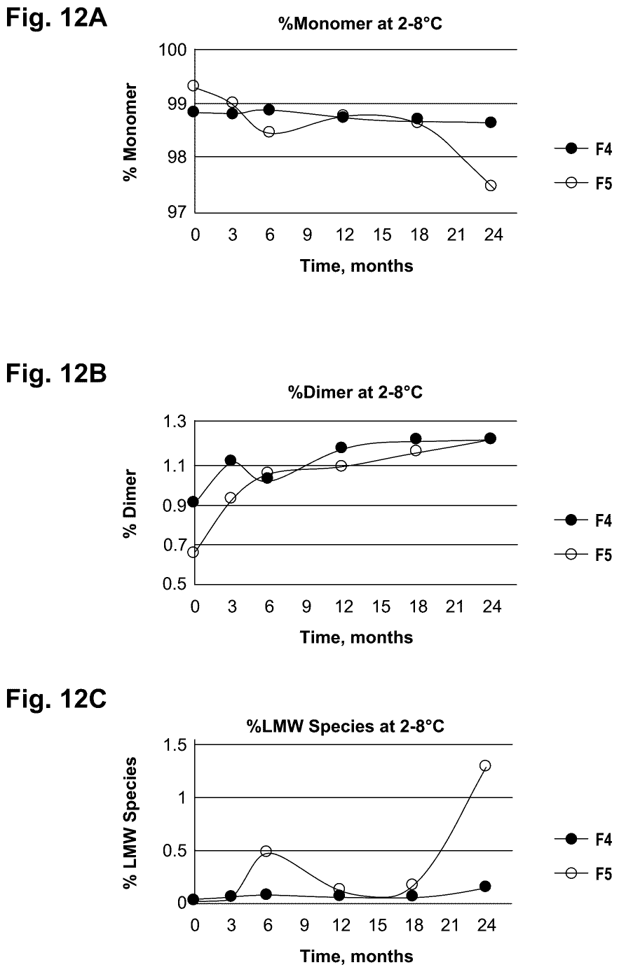

Table 29 and FIGS. 12A-12C show the percent (%) monomer, % dimer and % low molecular weight species of Formulations 4 and 5 measured by SEC. Both formulations stored at the long-term storage conditions of 2-8° C. met the acceptance criteria of % monomer and % dimer levels for up to 24 months. A slight decrease in % monomer with concurrent increase in % low molecular weight species was observed for Formulation 5 compared to Formulation 4.

TABLE 29
% Monomer, % Dimer and % Low Molecular
Weight Species at 2-8° C.

| | Formulation 4 | | | Formulation 5 | | |
|---|---|---|---|---|---|---|
| | % | % | | Monomer | % | |
| | Monomer | Dimer | | % | Dimer | |
| | (Accept- | (Accep- | | (Accept- | (Accept- | |
| | ance | ance | | ance | ance | |
| Time | criteria: | criteria: | % | criteria: | criteria: | % |
| (Months) | ≥95.0%) | ≤5.0%) | LMW | ≥95.0%) | ≤5.0%) | LMW |
| 0 | 98.85 | 0.91 | 0.04 | 99.32 | 0.65 | 0.02 |
| 1 | NT | NT | NT | NT | NT | NT |
| 3 | 98.81 | 1.11 | 0.05 | 99.01 | 0.92 | 0.05 |
| 6 | 98.88 | 1.02 | 0.07 | 98.45 | 1.05 | 0.47 |
| 9 | NT | NT | NT | NT | NT | NT |
| 12 | 98.73 | 1.18 | 0.05 | 98.77 | 1.09 | 0.12 |
| 18 | 98.69 | 1.22 | 0.05 | 98.64 | 1.16 | 0.16 |
| 24 | 98.63 | 1.21 | 0.13 | 97.45 | 1.22 | 1.29 |

LMW: Low molecular weight species;
NT: Not tested

### Capillary Gel Electrophoresis (Reducing and Non-reducing)

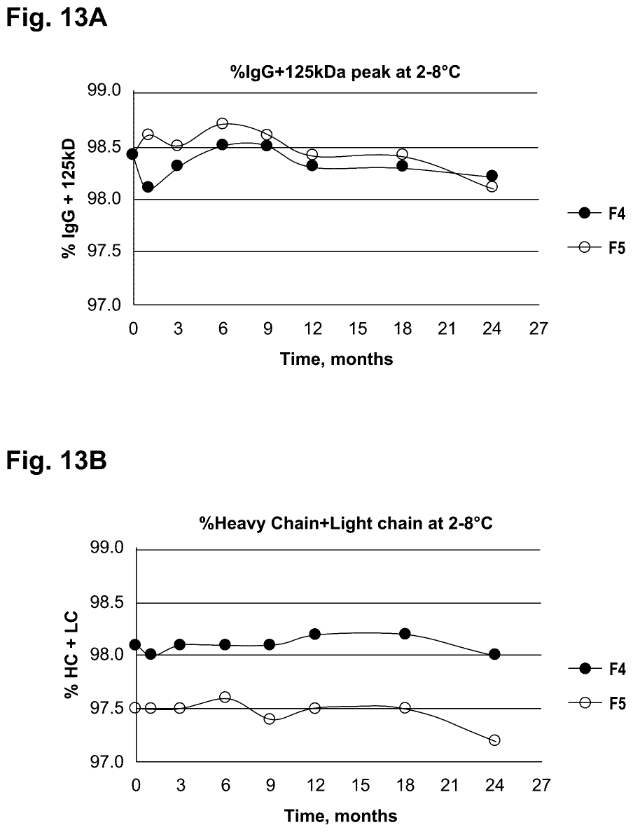

The percent (%) immunoglobulin G (IgG)+125 kDa peak was determined for Formulations 4 and 5 using non-reducing capillary gel electrophoresis (CGE). In addition, the % heavy chain+light chain was determined for Formulations 4 and 5 using reducing CGE. These results are shown in Tables 30-31 and FIGS. 13A-13B, respectively. In both formulations stored at the long-term storage conditions of 2-8° C., the % purity met acceptance criteria for up to 24 months.

TABLE 30
% IgG + 125 kDa Peak Measured Using Non-Reducing
CGE for Formulations 4 and 5 at 2-8° C.

| | | |
|---|---|---|
| Time | F4 | F5 |

| | |
|---|---|
| (Months) | 2-8° C.; Acceptance criteria: ≥90.0% |

| | | |
|---|---|---|
| 0 | 98.4 | 98.4 |
| 1 | 98.1 | 98.6 |
| 3 | 98.3 | 98.5 |
| 6 | 98.5 | 98.7 |
| 9 | 98.5 | 98.6 |
| 12 | 98.3 | 98.4 |
| 18 | 98.3 | 98.4 |
| 24 | 98.2 | 98.1 |

TABLE 31
% Heavy Chain and Light Chain Measured Using Reducing
CGE for Formulations 4 and 5 at 2-8° C.

| | | |
|---|---|---|
| Time | F4 | F5 |

| | |
|---|---|
| (Months) | 2-8° C.; Acceptance criteria: ≥90.0% |

| | | |
|---|---|---|
| 0 | 98.1 | 97.5 |
| 1 | 98.0 | 97.5 |
| 3 | 98.1 | 97.5 |
| 6 | 98.1 | 97.6 |
| 9 | 98.1 | 97.4 |
| 12 | 98.2 | 97.5 |
| 18 | 98.2 | 97.5 |
| 24 | 98.0 | 97.2 |

Capillary Isoelectric Focusing (icIEF)

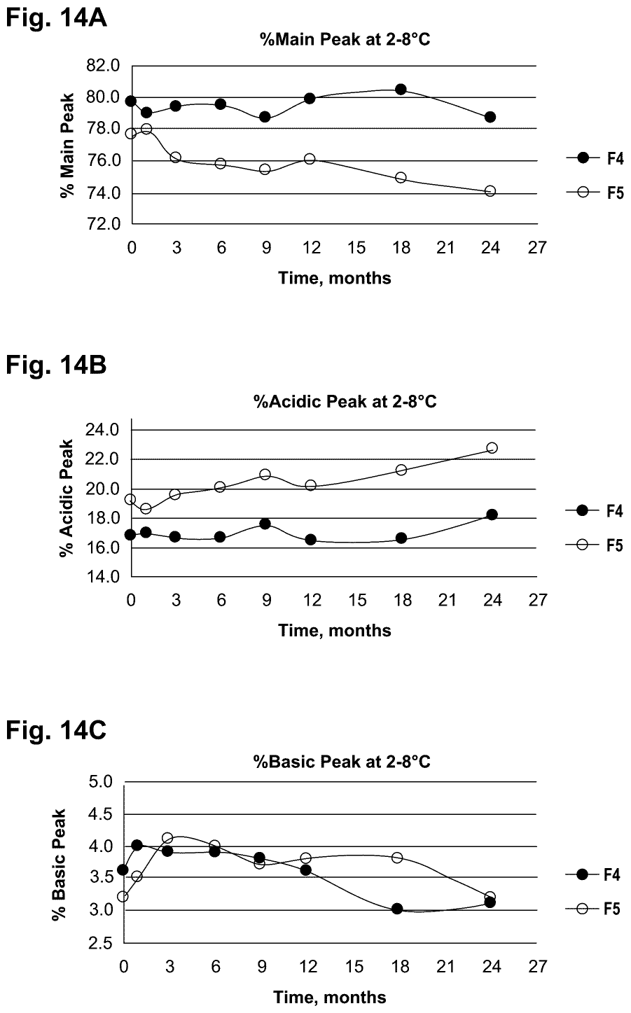

The percent (%) content of main species (main peak), % acidic species (acidic peak), and % basic species (basic peak) of Formulations 4 and 5 (F4 and F5) were measured using capillary isoelectric focusing (icIEF). The results are shown in Table 32. An overlay of stability trends in charge heterogeneity is presented in FIGS. 14A-14C. At the long-term storage conditions of 2-8° C. (FIGS. 14A, 14B, and 14C), both formulations (F4 and F5) met acceptance criteria for 24 months.

TABLE 32
% Main Peak, % Acidic Species and % Basic Species Determined Using icIEF for
Formulations 1 and 2 at 2-8° C.

| | Formulation 4 at 2-8° C. | | | Formulation 5 at 2-8° C. | | |
|---|---|---|---|---|---|---|
| | % Main | % Acidic | % Basic | % Main | % Acidic | % Basic |
| | peak | species | species | peak | species | species |
| | (Acceptance | (Acceptance | (Acceptance | (Acceptance | (Acceptance | (Acceptance |
| Time | criteria: | criteria: | criteria: | criteria: | criteria: | criteria: |
| (Months) | 50-90%) | 10-40%) | 0-10%) | 50-90%) | 10-40%) | 0-10%) |
| 0.0 | 79.7 | 16.8 | 3.6 | 77.6 | 19.2 | 3.2 |
| 1.0 | 79.0 | 17.0 | 4.0 | 77.9 | 18.6 | 3.5 |
| 3.0 | 79.4 | 16.7 | 3.9 | 76.2 | 19.6 | 4.1 |
| 6.0 | 79.5 | 16.7 | 3.9 | 75.8 | 20.1 | 4.0 |
| 9.0 | 78.7 | 17.5 | 3.8 | 75.4 | 20.9 | 3.7 |
| 12.0 | 79.9 | 16.5 | 3.6 | 76.1 | 20.2 | 3.8 |
| 18.0 | 80.4 | 16.6 | 3.0 | 74.9 | 21.3 | 3.8 |
| 24.0 | 78.7 | 18.2 | 3.1 | 74.1 | 22.7 | 3.2 |

### Sub-Visible Particles Using Micro-Flow Imaging (MFI)

Sub-visible particles in Formulations 4 and 5 were measured at different time points at the long-term storage condition of 2-8° C. The results are shown in Table 33. No significant changes in sub-visible particles were observed. Overall, sub-visible particles in the size range of ≥ (more than or equal to) 10 m were less than 6,000 particles/mL. In the size range of ≥ (more than or equal to) 25 m, the sub-visible particles were less than 600 particles/mL and were well within USP<788> limits even considering the increased sensitivity of using MFI for sub-visible particle detection. This data indicates that the 200 mg/mL formulation (F5) does not have a significant impact on sub-visible particle count at the long-term-storage condition.

TABLE 33
Sub-Visible Particles for Formulations 4 and 5 at 2-8° C.

| | SbVPs ≥2 μm | | Sb VPs ≥10 μm | | SbVPs ≥25 μm | |
|---|---|---|---|---|---|---|
| | (particles/mL) | | (particles/mL) | | (particles/mL) | |
| Time | at 2-8° C. | | at 2-8° C. | | at 2-8° C. | |
| (Months) | F4 | F5 | F4 | F5 | F4 | F5 |
| 0 | 32581 | 2658 | 417 | 404 | 65 | 57 |
| 1 | 31819 | NT | 262 | NT | 13 | NT |
| 3 | 33888 | 4513 | 299 | 354 | 39 | 57 |

SbVPs: Sub-visible particles;
NT: Not tested

### Chemical Modifications to Primary Structure Using Peptide Mapping

Amino acids that could potentially undergo chemical modification over time and influence protein structure were monitored for Formulations 4 and 5. Specifically, amino acid residues methionine 81 and methionine 254 of TEV-48574 could potentially undergo oxidation, affecting the primary structure, and were monitored. Similarly, asparagine 317 of TEV-48574 could potentially undergo deamidation, resulting in succinimide, and was monitored. As shown in Table 34, no significant changes were observed in % Met81 and % Met254 oxidation over time at the long-term storage condition of 2-8° C. for 24 months. At the 24M timepoint, the liquid drug product (F5) showed higher levels of Asn317 deamidation compared to the lyophilized drug product (F4). This difference in the extent of Asn deamidation may be the result of lower stability of the liquid drug product compared to the lyophilized drug product, and may not be attributable to the high nominal protein concentration in F5.

TABLE 34
% Met81 Oxidation, % Asn317 Deamidation, and %
Met254 Oxidation in Formulations 4 and 5

| | % Met81 | | % Asn317 | | % Met254 | |
|---|---|---|---|---|---|---|
| | Oxidation at 2- | | deamidation | | Oxidation | |
| Time | 8° C. | | at 2-8° C. | | at 2- 8° C. | |
| (Months) | F4 | F5 | F4 | F5 | F4 | F5 |
| 0 | 0.67 | 0.49 | 6.80 | 7.01 | 1.79 | 1.85 |
| 1 | 0.46 | 0.60 | 8.22 | 6.62 | 1.56 | 2.07 |
| 3 | 1.44 | 0.87 | 6.08 | 6.02 | 2.73 | 2.47 |
| 6 | 1.25 | 0.80 | 6.54 | 7.48 | 2.68 | 2.51 |
| 12 | 0.94 | 0.51 | 6.64 | 4.63 | 2.19 | 2.11 |
| 18 | 0.87 | 0.77 | 6.66 | 6.22 | 2.35 | 2.90 |
| 24 | 0.4 | 1.0 | 7.6 | 11.7 | 2.3 | 3.8 |

F4: Formulation 4;
F5: Formulation 5;
Met81: methionine residue 81 of TEV-48574;
Asn317: asparagine residue 317 of TEV-48574;
Met254: methionine residue 254 of TEV-48574

### Potency by ELISA

The percent (%) potency of Formulations 4 and 5 was determined by enzyme-linked immunosorbent assay (ELISA). The results are shown in Table 35. No significant changes were observed between the formulations at the long-term storage condition (2-8° C.), and the acceptance criteria of 70%-135% potency was met for 24 months.

TABLE 35
% Relative Potency of Formulations 4 and 5 by ELISA

| | |
|---|---|
| | % Potency at 2-8° C. |
| Time | Acceptance criteria: 70-135% |

| (Months) | F4 | F5 |
|---|---|---|
| 0 | 110 | 113 |
| 1 | 118 | 112 |
| 3 | 108 | 100 |
| 6 | 99 | 96 |
| 9 | 107 | 108 |
| 12 | 106 | 99 |
| 18 | 100 | 92 |
| 24 | 98 | 92 |

### Secondary and Tertiary Protein Structure Analysis Using Circular Dichroism (CD) Spectroscopy

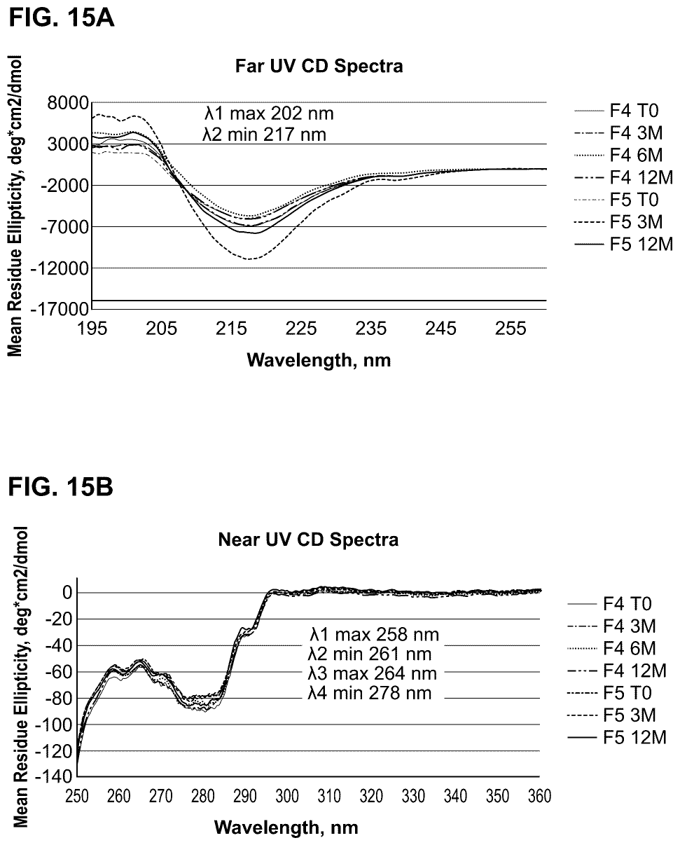

Secondary protein structure of Formulations 4 and 5 was analyzed using far-ultraviolet (far-UV) circular dichroism (CD), and tertiary protein structure of Formulations 4 and 5 was analyzed using near-UV CD. Secondary structure analysis was performed for time zero (T0) samples, and 3 month, 6 month, and 12 month samples of Formulation 4 at 25° C.; and for T0 samples, and 3 month and 12 month samples of Formulation 5 at 25° C. Tertiary structure analysis was performed for time zero (T0) samples, and 3 month and 12 month samples of Formulation 4 at 25° C.; and for T0 samples, and 3 month, 6 month and 12 month samples of Formulation 5 at 25° C. The results are shown in FIGS. 15A-15B (FIGS. 15A-15B). Specifically, FIG. 15A shows the secondary structure of Formulation 4 (F4) at time zero (T0) and after 3 months (3M), 6 months (6M) and 12 months (12M) of storage at 25° C., and of Formulation 5 (F5) at T0, and after 3M and 12M of storage at 25° C. using far UV CD. FIG. 15B shows the tertiary structure of F4 at T0, and after 3M and 12M of storage at 25° C., and of F5 at T0, and after 3M, 6M and 12M of storage at 25° C. using near UV CD. No significant change in secondary structure or tertiary structure was observed for either the lyophilized formulation (F4) or high concentration liquid formulation (F5).

### Conclusions

The stability of TEV-48574 in lyophilized form (Formulation 4 at 100 mg/mL) was comparable to a liquid formulation at 200 mg/mL (Formulation 5). At the long-term storage condition of 2-8° C., the drug product stability data for both formulations were observed to be comparable.

At the 24M timepoint, the liquid drug product showed higher levels of Asn317 deamidation compared to the lyophilized drug product. This difference in the extent of Asn deamidation may be the result of lower stability of the liquid drug product compared to the lyophilized drug product, and may not be attributable to the high nominal protein concentration in F2. A slight decrease in % monomer with concurrent increase in % low molecular weight species was also observed for F5 compared to F4. However, lyophilized formulations are typically more stable compared to liquid formulations.

Based on these data, the 200 mg/mL high concentration liquid drug product was observed to be stable at the 2-8° C., with no significant impacts on critical quality attributes of TEV-48574.

### Example 4: Evaluation of Arginine-HCl as an Excipient in TEV-48574 Liquid Formulation

The purpose of this study was to evaluate the effectiveness of Arginine-HCl (Arg-HCl) as an excipient and its impact on TEV-48574 drug product stability. Four liquid formulations were tested. Two formulations were prepared in 10 mM Histidine, 5% (w/v) Sucrose, 100 mM Arg-HCl, 0.02% (w/v) PS-80 at pH 6.0 with drug product at either 150 mg/mL (Formulation 1A) or 100 mg/mL (Formulation 2A). Two additional formulations were prepared in 10 mM Histidine, 5% (w/v) Sucrose, 0.02% (w/v) PS-80 at pH 6.0 with drug product at either 150 mg/mL (Formulation 1B) or 100 mg/mL (Formulation 2B). The stability of these formulations was evaluated at the stressed storage condition of 40° C. The impact of these conditions on product quality attributes was evaluated.

### Visual Appearance, Protein Concentration, Osmolality, and Viscosity

The results of a visual appearance, protein concentration, and osmolality analysis for Formulations 1A-2A and 1B-2B are shown in Tables 36-38. At the stressed storage condition of 40° C., the visual appearance for formulations 1A and 1B was observed to be slightly yellow, potentially related to degradation of these formulations at elevated temperatures over time. The measured protein concentration for the 4 formulations at 40° C. were close to the nominal concentration expected and did not vary over time. Osmolality and viscosity of the drug products was evaluated, and no significant difference was observed over time.

TABLE 36
Visual Appearance Results at 40° C.

| Time | Visual Appearance | | | |
|---|---|---|---|---|
| Point | 1A | 1B | 2A | 2B |
| T0 | L; FFVP; S | L; FFVP; S | L; FFVP; S | L; FFVP; S |
| 1 WK | L; FFVP; O | L; FFVP; S | L; FFVP; S | L; FFVP; S |
| 2 WK | L; FFVP; O | L; FFVP; O | L; FFVP; O | L; FFVP; O |
| 3 WK | L; 1 fibrous | L; FFVP; O | L; FFVP; S | L; FFVP; S |
| | particle; O | | | |
| 4 WK | L; FFVP; O | L; FFVP; S | L; FFVP; S | L; FFVP; S |
| 6 WK | L; FFVP; O | L; FFVP; O | L; FFVP; O | L; FFVP; O |
| 8 WK | VSY; FFVP; O | SY; FFVP; O | L; FFVP; O | L; FFVP; O |

T0: Time zero;
WK: Weeks;
1A: Formulation 1A;
1B: Formulation 1B;
2A: Formulation 2A;
2B: Formulation 2B;
L: Colorless;
FFVP: Free from visible particles;
O: Opaslescent;
SY: Slightly yellow;
VSY: Very slightly yellow;
S: Slightly opalescent

TABLE 37
Protein Concentration Results at 40° C.

| | |
|---|---|
| Time | Protein concentration (mg/mL) |

| Point | 1A | 1B | 2A | 2B |
|---|---|---|---|---|
| T0 | 152.4 | 150.7 | 102.7 | 104.9 |
| 1 WK | 150.7 | 152.2 | 102.8 | 111.6 |
| 2 WK | 150.3 | 149.0 | 103.1 | 108.5 |
| 3 WK | 150.8 | 150.1 | 103.7 | 108.4 |
| 4 WK | 148.7 | 150.2 | 107.7 | 107.8 |
| 6 WK | 147.5 | 148.5 | 101.4 | 107.5 |
| 8 WK | 148.4 | 150.8 | 104.7 | 106.5 |

T0: Time zero;
WK: Weeks;
1A: Formulation 1A;
1B: Formulation 1B;
2A: Formulation 2A;
2B: Formulation 2B

TABLE 38
Osmolality Results at 40° C.

| | |
|---|---|
| Time | Osmolality (mOsm/kg) |

| Point | 1A | 1B | 2A | 2B |
|---|---|---|---|---|
| T0 | 444 | 222 | 365 | 179 |
| 1 WK | 427 | 231 | 359 | 180 |
| 2 WK | 416 | 226 | 362 | 182 |
| 3 WK | 423 | 216 | 366 | 181 |
| 4 WK | 427 | 234 | 366 | 182 |
| 6 WK | 420 | 223 | 373 | 189 |
| 8 WK | 434 | 225 | 359 | 189 |

T0: Time zero;
WK: Weeks;
1A: Formulation 1A;
1B: Formulation 1B;
2A: Formulation 2A;
2B: Formulation 2B

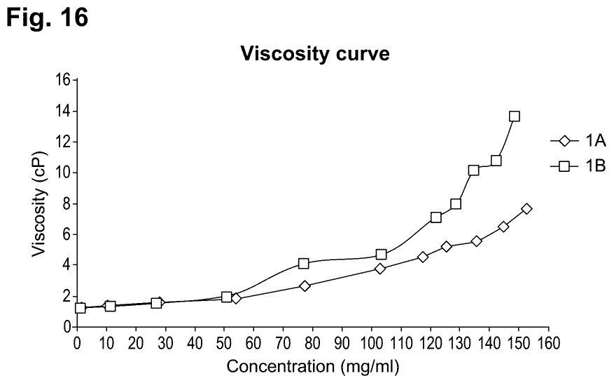

Osmolality of formulations 1A and 2A, which contain Arg-HCl, was higher compared to formulations 1B and 2B, which do not contain Arg-HCl. The viscosity for the formulation without Arg-HCl (1B) was observed to be higher than the viscosity of the formulation with Arg-HCl (1A) at 150 mg/mL as shown in Table 39 and FIG. 16.

TABLE 39
Viscocity for Formulations 1A and 1B at 20° C.

Viscosity (cP) measured at 20° C.

| Time Point | 1A | 1B |
|---|---|---|
| T0 | 7.72 | 13.68 |

T0: Time zero;
1A: Formulation 1A;
1B: Formulation 1B

### Size Exclusion Chromatography (SEC)

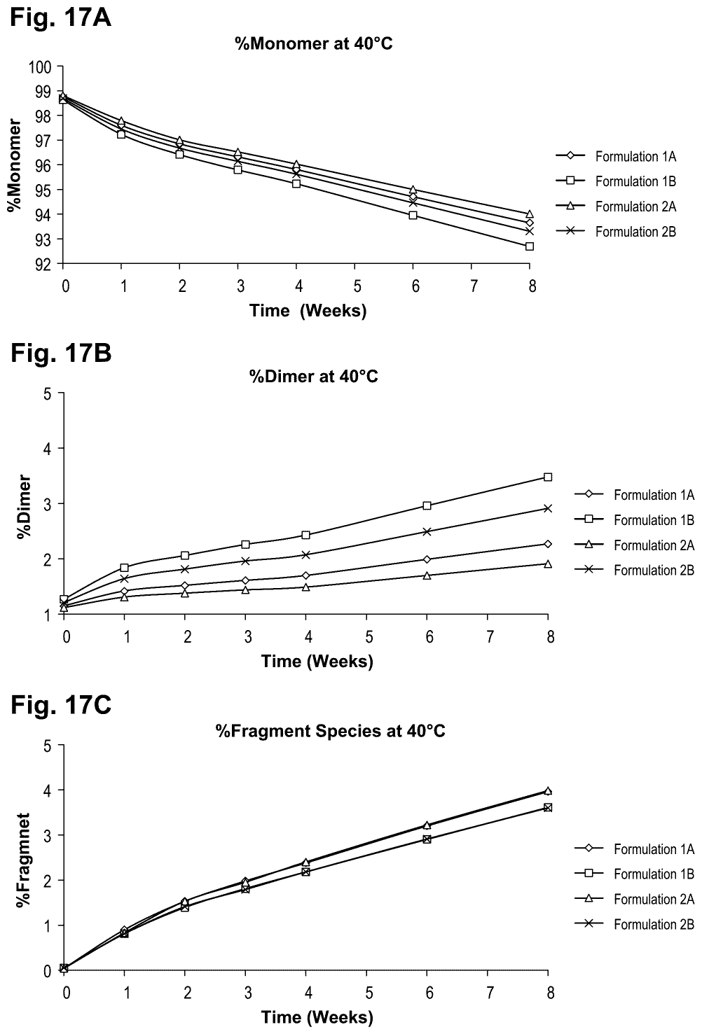

Tables 40-41 and FIGS. 17A-17C show the percent (%) monomer, % dimer and % fragment species of Formulations 1A-1B and 2A-2B measured by SEC after storage at 40° C. for up to 8 weeks. No significant difference in % monomer or % fragment was observed across the 4 formulations, as the reported values are within the method variability. Formulations 1B and 2B, which do not contain Arg-HCl, showed an increased rate of dimer formation compared to formulations 1A and 2A, which contain Arg-HCl, indicating a stabilizing effect of Arg-HCl on the drug product.

TABLE 40
% Monomer, % Dimer and % Fragment Species for
Formulations 1A-1B at 40° C.

| | Formulation 1A | | | Formulation 1B | | |
|---|---|---|---|---|---|---|
| Time | % | % | % | % | % | % |
| Point | Monomer | Dimer | Fragment | Monomer | Dimer | Fragment |
| T0 | 98.8 | 1.2 | 0.0 | 98.6 | 1.3 | 0.0 |
| 1 WK | 97.6 | 1.4 | 0.9 | 97.2 | 1.9 | 0.8 |
| 2 WK | 96.9 | 1.5 | 1.4 | 96.4 | 2.1 | 1.4 |
| 3 WK | 96.3 | 1.6 | 1.9 | 95.8 | 2.3 | 1.8 |
| 4 WK | 95.8 | 1.7 | 2.4 | 95.2 | 2.4 | 2.2 |
| 6 WK | 94.7 | 2.0 | 3.2 | 93.9 | 2.9 | 2.9 |
| 8 WK | 93.7 | 2.3 | 3.9 | 92.7 | 3.5 | 3.6 |

T0: Time zero;
WK: Week(s)

TABLE 41
% Monomer, % Dimer and % Fragment Species for
Formulations 2A-2B at 40° C.

| | Formulation 2A | | | Formulation 2B | | |
|---|---|---|---|---|---|---|
| Time | % | % | % | % | % | % |
| Point | Monomer | Dimer | Fragment | Monomer | Dimer | Fragment |
| T0 | 98.8 | 1.1 | 0.0 | 98.7 | 1.2 | 0.0 |
| 1 WK | 97.8 | 1.3 | 0.8 | 97.4 | 1.6 | 0.8 |
| 2 WK | 97.0 | 1.4 | 1.5 | 96.7 | 1.8 | 1.4 |
| 3 WK | 96.5 | 1.4 | 1.9 | 96.1 | 1.9 | 1.8 |
| 4 WK | 96.0 | 1.5 | 2.4 | 95.6 | 2.1 | 2.2 |
| 6 WK | 95.0 | 1.7 | 3.2 | 94.5 | 2.5 | 2.9 |
| 8 WK | 94.0 | 1.9 | 3.9 | 93.3 | 2.9 | 3.6 |

T0: Time zero;
WK: Week(s)

### Capillary Gel Electrophoresis (Reducing and Non-Reducing)

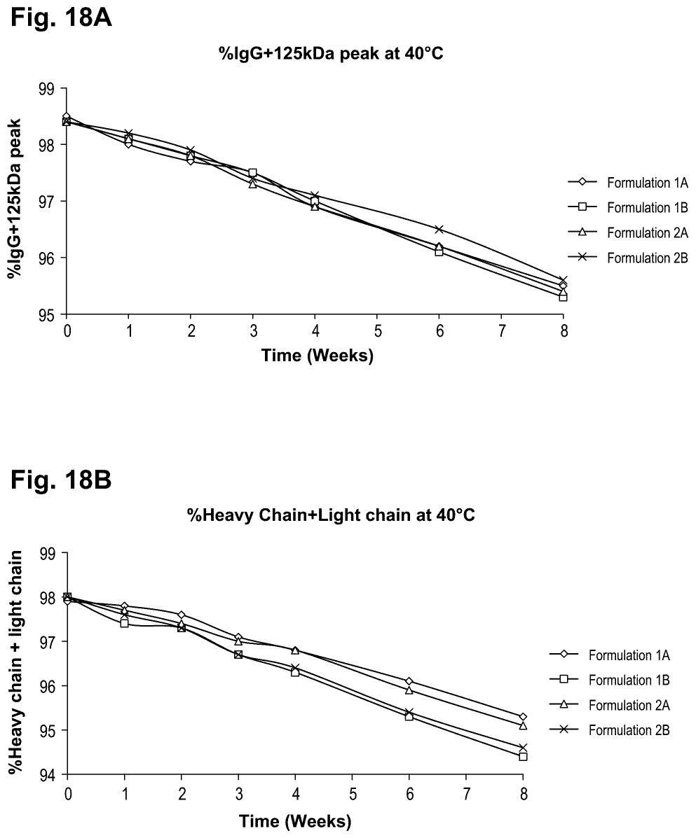

The percent (%) immunoglobulin G (IgG)+125 kDa peak was determined for Formulations 1A-1B and 2A-2B using non-reducing capillary gel electrophoresis (CGE) at the stressed condition of 40° C. for up to 8 weeks. In addition, the % heavy chain+light chain was determined for Formulations 1A-1B and 2A-2B using reducing CGE at the stressed condition of 40° C. for up to 8 weeks. These results are shown in Tables 42-43 and FIGS. 18A-18B, respectively. Formulations 1B and 2B, which lacked Arg-HCl, were observed to have increased rate of fragmentation compared to Formulations 1A and 2A, which contained Arg-HCl, demonstrating the stabilized effect of Arg-HCl on fragmentation pattern of the drug product.

TABLE 42
% IgG + 125 kDa Peak Measured Using Non-Reducing CGE
for Formulations 1A-1B and 2A-2B at 40° C.

| Time | Formulation | Formulation | Formulation | Formulation |
|---|---|---|---|---|
| Point | 1A | 1B | 2A | 2B |
| T0 | 98.5 | 98.4 | 98.4 | 98.4 |
| 1 WK | 98.0 | 98.1 | 98.1 | 98.2 |
| 2 WK | 97.7 | 97.8 | 97.8 | 97.9 |
| 3 WK | 97.5 | 97.5 | 97.3 | 97.4 |
| 4 WK | 96.9 | 97.0 | 96.9 | 97.1 |
| 6 WK | 96.2 | 96.1 | 96.2 | 96.5 |
| 8 WK | 95.5 | 95.3 | 95.4 | 95.6 |

T0: Time zero;
WK: Weeks

TABLE 43
% Heavy Chain and Light Chain Measured Using Reducing
CGE for Formulations 1A-1B and 2A-2B at 40° C.

| Time | Formulation | Formulation | Formulation | Formulation |
|---|---|---|---|---|
| Point | 1A | 1B | 2A | 2B |
| T0 | 97.9 | 98.0 | 98.0 | 98.0 |
| 1 WK | 97.8 | 97.4 | 97.7 | 97.6 |
| 2 WK | 97.6 | 97.3 | 97.4 | 97.3 |
| 3 WK | 97.1 | 96.7 | 97.0 | 96.7 |
| 4 WK | 96.8 | 96.3 | 96.8 | 96.4 |
| 6 WK | 96.1 | 95.3 | 95.9 | 95.4 |
| 8 WK | 95.3 | 94.4 | 95.1 | 94.6 |

T0: Time zero;
WK: Weeks

Capillary Isoelectric Focusing (icIEF)

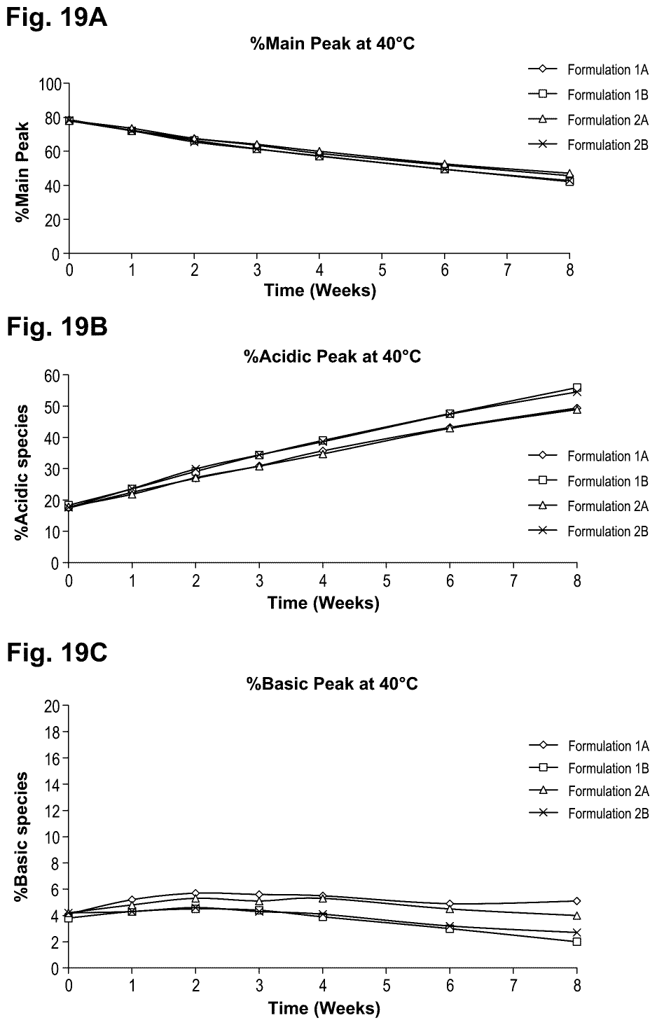

The percent (%) content of main species (main peak), % acidic species (acidic peak), and % basic species (basic peak) of Formulations 1A-1B and 2A-2B were measured using capillary isoelectric focusing (icIEF) at the stressed condition of 40° C. for up to 8 weeks. The results are shown in Tables 44-45. An overlay of stability trends in charge heterogeneity is presented in FIGS. 19A-19C. Slightly higher levels of acidic species were observed at the end of the 8 weeks for formulations 1B and 2B, which lacked Arg-HCl. This may be related to the higher rates of fragmentation evident for these formulations compared to formulations 1A and 2A, as reported in Table 43.

TABLE 44
% Main Peak, % Acidic Species and % Basic Species Determined
Using icIEF for Formulations 1A and 1B at 40° C.

| | Formulation 1A at 40° C. | | | Formulation 1B at 40° C. | | |
|---|---|---|---|---|---|---|
| Time | % Main | % Acidic | % Basic | % Main | % Acidic | % Basic |
| Point | peak | species | species | peak | species | species |
| T0 | 78.6 | 17.4 | 4.1 | 77.9 | 18.3 | 3.8 |
| 1 WK | 72.3 | 22.4 | 5.2 | 72.1 | 23.5 | 4.3 |
| 2 WK | 67.4 | 26.9 | 5.7 | 66.4 | 29.1 | 4.5 |
| 3 WK | 63.5 | 30.9 | 5.6 | 61.4 | 34.3 | 4.4 |
| 4 WK | 58.7 | 35.7 | 5.5 | 57.1 | 39.0 | 3.9 |
| 6 WK | 51.8 | 43.2 | 4.9 | 49.4 | 47.6 | 3.0 |
| 8 WK | 45.6 | 49.4 | 5.1 | 42.1 | 55.9 | 2.0 |

T0: Time zero;
WK: Week(s)

TABLE 45
% Main Peak, % Acidic Species and % Basic Species Determined
Using icIEF for Formulations 2A and 2B at 40° C.

| | Formulation 2A at 40° C. | | | Formulation 2B at 40° C. | | |
|---|---|---|---|---|---|---|
| Time | % Main | % Acidic | % Basic | % Main | % Acidic | % Basic |
| Point | peak | species | species | peak | species | species |
| T0 | 78.0 | 17.8 | 4.2 | 78.2 | 17.6 | 4.2 |
| 1 WK | 73.5 | 21.8 | 4.8 | 72.1 | 23.6 | 4.3 |
| 2 WK | 67.6 | 27.1 | 67.6 | 65.5 | 29.9 | 4.6 |
| 3 WK | 64.1 | 30.8 | 5.1 | 61.3 | 34.4 | 4.3 |
| 4 WK | 60.0 | 34.7 | 5.3 | 57.3 | 38.6 | 4.1 |
| 6 WK | 52.6 | 42.9 | 4.5 | 49.4 | 47.4 | 3.2 |
| 8 WK | 47.1 | 48.9 | 4.0 | 42.8 | 54.5 | 2.7 |

T0: Time zero;
WK: Week(s)

### Chemical Modifications to Primary Structure Using Peptide Mapping

Amino acids that could potentially undergo chemical modification over time and influence protein structure were monitored for Formulations 1A-1B and 2A-2B at the stressed condition of 40° C. for up to 8 weeks. Specifically, amino acid residues methionine 81 and methionine 254 of TEV-48574 could potentially undergo oxidation, affecting the primary structure, and were monitored. Similarly, asparagine 317 of TEV-48574 could potentially undergo deamidation, resulting in succinimide, and was monitored. No significant changes were observed in % Met81 oxidation, % Asn317 deamidation or % Met254 oxidation over time at the stressed storage condition of 40° C. for up to 8 weeks (data not shown), indicating that the presence of Arg-HCl does not appear to have an impact on this attribute.

### Potency by ELISA

The percent (%) potency of Formulations 1A-1B and 2A-2B was determined by enzyme-linked immunosorbent assay (ELISA) at the stressed condition of 40° C. for up to 8 weeks. The results are shown in Table 46. The trend of % potency was comparable across the formulations regardless of drug product concentration or the inclusion of Arg-HCl.

TABLE 46
% Relative Potency of Formulations 1A-1B and 2A-2B by ELISA

| Time | % Potency at 40° C. Acceptance criteria: 70-135% | | | |
|---|---|---|---|---|
| Point | 1A | 1B | 2A | 2B |
| T0 | 107% | 103% | 105% | 106% |
| 2 WK | 100% | 94% | 98% | 98% |
| 4 WK | 86% | 84% | 88% | 87% |
| 8 WK | 89% | 89% | 85% | 86% |

T0: Time zero;
WK: Weeks;
1A: Formulation 1A;
1B: Formulation 1B;
2A: Formulation 2A;
2B: Formulation 2B

### Dynamic Light Scattering (DLS) for Particle Size Characterization

DLS was employed to measure the % polydispersity (% PD) and the hydrodynamic radius of the drug product and nanoparticles that might be present in Formulations 1A-1B and 2A-2B. DLS analysis was performed for a time zero (T0) sample of Formulations 1A-1B and 2A-2B. Table 47 shows the results of the DLS analysis. It was observed that the apparent hydrodynamic radius for formulations 1B and 2B (without Arg-HCl) was smaller compared to formulations 1A and 2A (with Arg-HCl).

TABLE 47
% Polydispersity (%PD) and Radius of Particles for
Formulations 1A-1B and 2A-2B

| | Formulation | | Formulation | | Formulation | | Formulation | |
|---|---|---|---|---|---|---|---|---|
| | 1A | | 1B | | 2A | | 2B | |
| Time | Radius | % | Radius | % | Radius | % | Radius | % |
| Point | (nm) | PD | (nm) | PD | (nm) | PD | (nm) | PD |
| T0 | 8.9 | 7.1 | 6.5 | 10.8 | 8.2 | 4.0 | 5.1 | 7.5 |

T0: Time zero

### Differential Scanning Calorimetry (DSC) Analysis for Thermal Stability

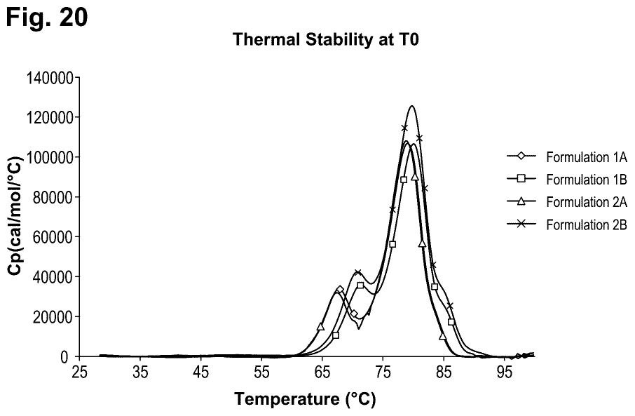

DSC was employed to evaluate thermal stability of Formulations 1A-1B and 2A-2B. DSC analysis was performed for a time zero (T0) sample of Formulations 1A-1B and 2A-2B. FIG. 20 shows the thermal stability of Formulations 1A-1B and 2A-2B at time zero (T0) and Table 48 shows the DSC thermogram for all 4 formulations. The Tₒₙₛₑₜ was about 60° C. for formulations 1A and 2A, and about 63° C. for formulations 1B and 2B. The transition temperature (Tm) was about 78° C. for all formulations.

TABLE 48
DSC Thermogram for Formulations 1A-1B and 2A-2B

| Formulation | Tm1 (° C.) | Tm2 (° C.) |
|---|---|---|
| 1A | 67.9 | 78.8 |
| 1B | 71.2 | 79.8 |
| 2A | 67.5 | 78.6 |
| 2B | 70.7 | 79.5 |

The Tm1 for Formulations 1A and 2A was found to be slightly shifted to lower temperatures, demonstrating that Arg-HCl may impact the thermal stability of the molecule. This could be due to protein fragmentation observed in the samples at these conditions, leading to a decrease in enthalpy. However, this temperature is above the 2-8° C. recommended storage condition for this drug product.

### Conclusions

The effectiveness of Arginine-HCl (Arg-HCl) as an excipient and its impact on TEV-48574 drug product stability was assessed across two different drug product concentrations (100 mg/mL and 150 mg/mL). For all 4 formulations tested, the quality attributes of % dimer and % purity measured using reduced CGE indicated stabilizing effects of 100 mM Arg-HCl compared to formulations without Arg-HCl. Viscosity of the drug product at 150 mg/mL with Arg-HCl was lower compared to the formulation without Arg-HCl. A small shift in the Tm1 analyzed by DSC for the formulations containing Arg-HCl of about 3° C. was observed. However, the recommend storage condition for the drug product is 2-8° C., so this observed shift in Tm1 is not expected to significantly impact stability during typical storage and handling conditions. All other attributes did not show significant change over 8 weeks at 40° C. across all 4 formulations.

Based on these data, the 100 mM Arg-HCl in the TEV-48574 formulation has demonstrated the potential to stabilize the formulation and minimize viscosity as the concentration of the drug product is increased.

It is to be appreciated that the Detailed Description section, and not the Summary and Abstract sections, is intended to be used to interpret the claims. The Summary and Abstract sections can set forth one or more but not all exemplary aspects of the present invention as contemplated by the inventor(s), and thus, are not intended to limit the present invention and the appended claims in any way.

The foregoing description of the specific aspects will so fully reveal the general nature of the invention that others can, by applying knowledge within the skill of the art, readily modify and/or adapt for various applications such specific aspects, without undue experimentation, without departing from the general concept of the present invention. Therefore, such adaptations and modifications are intended to be within the meaning and range of equivalents of the disclosed aspects, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present invention should not be limited by any of the above-described exemplary aspects, but should be defined only in accordance with the following claims and their equivalents.

Various publications, including patents, published applications, accession numbers, technical articles and scholarly articles are cited throughout the specification. Each of these cited publications is incorporated by reference, in its entirety and for all purposes, in this document.

Sequence Table

| SEQ | | |
|---|---|---|
| ID NO: | Description | Sequence |
| 1 | 320-587 Heavy | GYTFTSYDIN |
| | Chain CDR1 | |
| 2 | 320-587 Heavy | WLNPNSGYTG |
| | Chain CDR2 | |
| 3 | 320-587 Heavy | EVPETAAFEY |
| | Chain CDR3 | |
| 4 | 320-587 Light Chain | TSSSSDIGAGLGVH |
| | CDR1 | |
| 5 | 320-587 Light Chain | GYYNRPS |
| | CDR2 | |
| 6 | 320-587 Light Chain | QSWDGTLSAL |
| | CDR3 | |
| 7 | 320-587 Heavy | QVQLVQSGAEVKKPGASVKVSCKASGYTFTSYDINWVRQ |
| | Chain Variable | APGQGLEWMGWLNPNSGYTGYAQKFQGRVTMTADRST |
| | Region | STAYMELSSLRSEDTAVYYCAREVPETAAFEYWGQGTLVT |
| | | VSS |
| 8 | 320-587 Light Chain | QSVLTQPPSVSGAPGQRVTISCTSSSSDIGAGLGVHWYQQ |
| | Variable Region | LPGTAPKLLIEGYYNRPSGVPDRFSGSKSGTSASLTITGLLPE |
| | | DEGDYYCQSWDGTLSALFGGGTKLTVLG |
| 9 | 320-587 Heavy | QVQLVQSGAEVKKPGASVKVSCKASGYTFTSYDINWVRQ |
| | Chain | APGQGLEWMGWLNPNSGYTGYAQKFQGRVTMTADRST |
| | | STAYMELSSLRSEDTAVYYCAREVPETAAFEYWGQGTLVT |
| | | VSSASTKGPSVFPLAPSSKSTSGGTAALGCLVKDYFPEPVTV |
| | | SWNSGALTSGVHTFPAVLQSSGLYSLSSVVTVPSSSLGTQT |
| | | YICNVNHKPSNTKVDKKVEPKSCDKTHTCPPCPAPELLGGP |
| | | SVFLFPPKPKDTLMISRTPEVTCVVVDVSHEDPEVKFNWYV |
| | | DGVEVHNAKTKPREEQYNSTYRVVSVLTVLHQDWLNGKE |
| | | YKCKVSNKALPAPIEKTISKAKGQPREPQVYTLPPSRDELTK |
| | | NQVSLTCLVKGFYPSDIAVEWESNGQPENNYKTTPPVLDS |
| | | DGSFFLYSKLTVDKSRWQQGNVFSCSVMHEALHNHYTQK |
| | | SLSLSPG |
| 10 | 320-587 Light Chain | QSVLTQPPSVSGAPGQRVTISCTSSSSDIGAGLGVHWYQQ |
| | | LPGTAPKLLIEGYYNRPSGVPDRFSGSKSGTSASLTITGLLPE |
| | | DEGDYYCQSWDGTLSALFGGGTKLTVLGQPKAAPSVTLFP |
| | | PSSEELQANKATLVCLISDFYPGAVTVAWKADSSPVKAGVE |
| | | TTTPSKQSNNKYAASSYLSLTPEQWKSHRSYSCQVTHEGST |
| | | VEKTVAPTECS |
| 11 | 320-179 Heavy | QVQLVQSGAEVKKPGASVKVSCKASGYTFTSYDINWVRQ |
| | Chain Variable | APGQGLEWMGWLNPNSGNTGYAQKFQGRVTMTADRS |
| | Region | TSTAYMELSSLRSEDTAVYYCAREVPETAAFEYWGQGTLV |
| | | TVSS |
| 12 | 320-179 Light Chain | QSVLTQPPSVSGAPGQRVTISCTSSSSDIGAGLGVHWYQQ |
| | Variable Region | LPGTAPKLLIEGYYNRPSGVPDRFSGSKSGTSASLTITGLLPE |
| | | DEGDYYCQSYDGTLSALFGGGTKLTVLG |
| 13 | IgG1 HC constant | ASTKGPSVFPLAPSSKSTSGGTAALGCLVKDYFPEPVTVSW |
| | region | NSGALTSGVHTFPAVLQSSGLYSLSSVVTVPSSSLGTQTYIC |
| | | NVNHKPSNTKVDKKVEPKSCDKTHTCPPCPAPELLGGPSV |
| | | FLFPPKPKDTLMISRTPEVTCVVVDVSHEDPEVKFNWYVD |
| | | GVEVHNAKTKPREEQYNSTYRVVSVLTVLHQDWLNGKEY |
| | | KCKVSNKALPAPIEKTISKAKGQPREPQVYTLPPSRDELTKN |
| | | QVSLTCLVKGFYPSDIAVEWESNGQPENNYKTTPPVLDSD |
| | | GSFFLYSKLTVDKSRWQQGNVFSCSVMHEALHNHYTQKS |
| | | LSLSPGK |
| 14 | IgG1 dK HC constant | ASTKGPSVFPLAPSSKSTSGGTAALGCLVKDYFPEPVTVSW |
| | region | NSGALTSGVHTFPAVLQSSGLYSLSSVVTVPSSSLGTQTYIC |
| | | NVNHKPSNTKVDKKVEPKSCDKTHTCPPCPAPELLGGPSV |
| | | FLFPPKPKDTLMISRTPEVTCVVVDVSHEDPEVKFNWYVD |
| | | GVEVHNAKTKPREEQYNSTYRVVSVLTVLHQDWLNGKEY |
| | | KCKVSNKALPAPIEKTISKAKGQPREPQVYTLPPSRDELTKN |
| | | QVSLTCLVKGFYPSDIAVEWESNGQPENNYKTTPPVLDSD |
| | | GSFFLYSKLTVDKSRWQQGNVFSCSVMHEALHNHYTQKS |
| | | LSLSPG |
| 15 | IgG1 | ASTKGPSVFPLAPSSKSTSGGTAALGCLVKDYFPEPVTVSW |
| | 252Y/254T/256E HC | NSGALTSGVHTFPAVLQSSGLYSLSSVVTVPSSSLGTQTYIC |
| | constant region | NVNHKPSNTKVDKKVEPKSCDKTHTCPPCPAPELLGGPSV |
| | | FLFPPKPKDTLYITREPEVTCVVVDVSHEDPEVKFNWYVDG |
| | | VEVHNAKTKPREEQYNSTYRVVSVLTVLHQDWLNGKEYK |
| | | CKVSNKALPAPIEKTISKAKGQPREPQVYTLPPSRDELTKNQ |
| | | VSLTCLVKGFYPSDIAVEWESNGQPENNYKTTPPVLDSDGS |
| | | FFLYSKLTVDKSRWQQGNVFSCSVMHEALHNHYTQKSLSL |
| | | SPGK |
| 16 | IgG1 dK + | ASTKGPSVFPLAPSSKSTSGGTAALGCLVKDYFPEPVTVSW |
| | 252Y/254T/256E HC | NSGALTSGVHTFPAVLQSSGLYSLSSVVTVPSSSLGTQTYIC |
| | constant region | NVNHKPSNTKVDKKVEPKSCDKTHTCPPCPAPELLGGPSV |
| | | FLFPPKPKDTLYITREPEVTCVVVDVSHEDPEVKFNWYVDG |
| | | VEVHNAKTKPREEQYNSTYRVVSVLTVLHQDWLNGKEYK |
| | | CKVSNKALPAPIEKTISKAKGQPREPQVYTLPPSRDELTKNQ |
| | | VSLTCLVKGFYPSDIAVEWESNGQPENNYKTTPPVLDSDGS |
| | | FFLYSKLTVDKSRWQQGNVFSCSVMHEALHNHYTQKSLSL |
| | | SPG |
| 17 | IgG1 L234A, L235A, | ASTKGPSVFPLAPSSKSTSGGTAALGCLVKDYFPEPVTVSW |
| | G237A HC constant | NSGALTSGVHTFPAVLQSSGLYSLSSVVTVPSSSLGTQTYIC |
| | region | NVNHKPSNTKVDKKVEPKSCDKTHTCPPCPAPEAAGAPSV |
| | | FLFPPKPKDTLMISRTPEVTCVVVDVSHEDPEVKFNWYVD |
| | | GVEVHNAKTKPREEQYNSTYRVVSVLTVLHQDWLNGKEY |
| | | KCKVSNKALPAPIEKTISKAKGQPREPQVYTLPPSREEMTK |
| | | NQVSLTCLVKGFYPSDIAVEWESNGQPENNYKTTPPVLDS |
| | | DGSFFLYSKLTVDKSRWQQGNVFSCSVMHEALHNHYTQK |
| | | SLSLSPGK |
| 18 | IgG1 dK + L234A, | ASTKGPSVFPLAPSSKSTSGGTAALGCLVKDYFPEPVTVSW |
| | L235A, G237A HC | NSGALTSGVHTFPAVLQSSGLYSLSSVVTVPSSSLGTQTYIC |
| | constant region | NVNHKPSNTKVDKKVEPKSCDKTHTCPPCPAPEAAGAPSV |
| | | FLFPPKPKDTLMISRTPEVTCVVVDVSHEDPEVKFNWYVD |
| | | GVEVHNAKTKPREEQYNSTYRVVSVLTVLHQDWLNGKEY |
| | | KCKVSNKALPAPIEKTISKAKGQPREPQVYTLPPSREEMTK |
| | | NQVSLTCLVKGFYPSDIAVEWESNGQPENNYKTTPPVLDS |
| | | DGSFFLYSKLTVDKSRWQQGNVFSCSVMHEALHNHYTQK |
| | | SLSLSPG |
| 19 | IgG1 L235A/G237A | ASTKGPSVFPLAPSSKSTSGGTAALGCLVKDYFPEPVTVSW |
| | HC constant region | NSGALTSGVHTFPAVLQSSGLYSLSSVVTVPSSSLGTQTYIC |
| | | NVNHKPSNTKVDKKVEPKSCDKTHTCPPCPAPELAGAPSV |
| | | FLFPPKPKDTLMISRTPEVTCVVVDVSHEDPEVKFNWYVD |
| | | GVEVHNAKTKPREEQYNSTYRVVSVLTVLHQDWLNGKEY |
| | | KCKVSNKALPAPIEKTISKAKGQPREPQVYTLPPSRDELTKN |
| | | QVSLTCLVKGFYPSDIAVEWESNGQPENNYKTTPPVLDSD |
| | | GSFFLYSKLTVDKSRWQQGNVFSCSVMHEALHNHYTQKS |
| | | LSLSPGK |
| 20 | IgG1 dK + | ASTKGPSVFPLAPSSKSTSGGTAALGCLVKDYFPEPVTVSW |
| | L235A/G237A HC | NSGALTSGVHTFPAVLQSSGLYSLSSVVTVPSSSLGTQTYIC |
| | constant region | NVNHKPSNTKVDKKVEPKSCDKTHTCPPCPAPELAGAPSV |
| | | FLFPPKPKDTLMISRTPEVTCVVVDVSHEDPEVKFNWYVD |
| | | GVEVHNAKTKPREEQYNSTYRVVSVLTVLHQDWLNGKEY |
| | | KCKVSNKALPAPIEKTISKAKGQPREPQVYTLPPSRDELTKN |
| | | QVSLTCLVKGFYPSDIAVEWESNGQPENNYKTTPPVLDSD |
| | | GSFFLYSKLTVDKSRWQQGNVFSCSVMHEALHNHYTQKS |
| | | LSLSPG |
| 21 | IgG2 HC constant | ASTKGPSVFPLAPCSRSTSESTAALGCLVKDYFPEPVTVSW |
| | region | NSGALTSGVHTFPAVLQSSGLYSLSSVVTVPSSNFGTQTYT |
| | | CNVDHKPSNTKVDKTVERKCCVECPPCPAPPVAGPSVFLF |
| | | PPKPKDTLMISRTPEVTCVVVDVSHEDPEVQFNWYVDGV |
| | | EVHNAKTKPREEQFNSTFRVVSVLTVVHQDWLNGKEYKC |
| | | KVSNKGLPAPIEKTISKTKGQPREPQVYTLPPSREEMTKNQ |
| | | VSLTCLVKGFYPSDIAVEWESNGQPENNYKTTPPMLDSDG |
| | | SFFLYSKLTVDKSRWQQGNVFSCSVMHEALHNHYTQKSLS |
| | | LSPGK |
| 22 | IgG2 dK HC constant | ASTKGPSVFPLAPCSRSTSESTAALGCLVKDYFPEPVTVSW |
| | region | NSGALTSGVHTFPAVLQSSGLYSLSSVVTVPSSNFGTQTYT |
| | | CNVDHKPSNTKVDKTVERKCCVECPPCPAPPVAGPSVFLF |
| | | PPKPKDTLMISRTPEVTCVVVDVSHEDPEVQFNWYVDGV |
| | | EVHNAKTKPREEQFNSTFRVVSVLTVVHQDWLNGKEYKC |
| | | KVSNKGLPAPIEKTISKTKGQPREPQVYTLPPSREEMTKNQ |
| | | VSLTCLVKGFYPSDIAVEWESNGQPENNYKTTPPMLDSDG |
| | | SFFLYSKLTVDKSRWQQGNVFSCSVMHEALHNHYTQKSLS |
| | | LSPG |
| 23 | IgG2 A330S/P331S | ASTKGPSVFPLAPCSRSTSESTAALGCLVKDYFPEPVTVSW |
| | HC constant region | NSGALTSGVHTFPAVLQSSGLYSLSSVVTVPSSNFGTQTYT |
| | | CNVDHKPSNTKVDKTVERKCCVECPPCPAPPVAGPSVFLF |
| | | PPKPKDTLMISRTPEVTCVVVDVSHEDPEVQFNWYVDGV |
| | | EVHNAKTKPREEQFNSTFRVVSVLTVVHQDWLNGKEYKC |
| | | KVSNKGLPSSIEKTISKTKGQPREPQVYTLPPSREEMTKNQ |
| | | VSLTCLVKGFYPSDIAVEWESNGQPENNYKTTPPMLDSDG |
| | | SFFLYSKLTVDKSRWQQGNVFSCSVMHEALHNHYTQKSLS |
| | | LSPGK |
| 24 | IgG2 dK + | ASTKGPSVFPLAPCSRSTSESTAALGCLVKDYFPEPVTVSW |
| | A330S/P331S HC | NSGALTSGVHTFPAVLQSSGLYSLSSVVTVPSSNFGTQTYT |
| | constant region | CNVDHKPSNTKVDKTVERKCCVECPPCPAPPVAGPSVFLF |
| | | PPKPKDTLMISRTPEVTCVVVDVSHEDPEVQFNWYVDGV |
| | | EVHNAKTKPREEQFNSTFRVVSVLTVVHQDWLNGKEYKC |
| | | KVSNKGLPSSIEKTISKTKGQPREPQVYTLPPSREEMTKNQ |
| | | VSLTCLVKGFYPSDIAVEWESNGQPENNYKTTPPMLDSDG |
| | | SFFLYSKLTVDKSRWQQGNVFSCSVMHEALHNHYTQKSLS |
| | | LSPG |
| 25 | IgG4 HC constant | ASTKGPSVFPLAPCSRSTSESTAALGCLVKDYFPEPVTVSW |
| | region | NSGALTSGVHTFPAVLQSSGLYSLSSVVTVPSSSLGTKTYTC |
| | | NVDHKPSNTKVDKRVESKYGPPCPPCPAPEFLGGPSVFLFP |
| | | PKPKDTLMISRTPEVTCVVVDVSQEDPEVQFNWYVDGVE |
| | | VHNAKTKPREEQFNSTYRVVSVLTVLHQDWLNGKEYKCK |
| | | VSNKGLPSSIEKTISKAKGQPREPQVYTLPPSQEEMTKNQV |
| | | SLTCLVKGFYPSDIAVEWESNGQPENNYKTTPPVLDSDGSF |
| | | FLYSRLTVDKSRWQEGNVFSCSVMHEALHNHYTQKSLSLS |
| | | LGK |
| 26 | IgG4 dK + S228P HC | ASTKGPSVFPLAPCSRSTSESTAALGCLVKDYFPEPVTVSW |
| | constant region | NSGALTSGVHTFPAVLQSSGLYSLSSVVTVPSSSLGTKTYTC |
| | | NVDHKPSNTKVDKRVESKYGPPCPPCPAPEFLGGPSVFLFP |
| | | PKPKDTLMISRTPEVTCVVVDVSQEDPEVQFNWYVDGVE |
| | | VHNAKTKPREEQFNSTYRVVSVLTVLHQDWLNGKEYKCK |
| | | VSNKGLPSSIEKTISKAKGQPREPQVYTLPPSQEEMTKNQV |
| | | SLTCLVKGFYPSDIAVEWESNGQPENNYKTTPPVLDSDGSF |
| | | FLYSRLTVDKSRWQEGNVFSCSVMHEALHNHYTQKSLSLS |
| | | LG |
| 27 | IgG4 S228P + | ASTKGPSVFPLAPCSRSTSESTAALGCLVKDYFPEPVTVSW |
| | 252Y/254T/256E HC | NSGALTSGVHTFPAVLQSSGLYSLSSVVTVPSSSLGTKTYTC |
| | constant region | NVDHKPSNTKVDKRVESKYGPPCPPCPAPEFLGGPSVFLFP |
| | | PKPKDTLYITREPEVTCVVVDVSQEDPEVQFNWYVDGVEV |
| | | HNAKTKPREEQFNSTYRVVSVLTVLHQDWLNGKEYKCKVS |
| | | NKGLPSSIEKTISKAKGQPREPQVYTLPPSQEEMTKNQVSL |
| | | TCLVKGFYPSDIAVEWESNGQPENNYKTTPPVLDSDGSFFL |
| | | YSRLTVDKSRWQEGNVFSCSVMHEALHNHYTQKSLSLSLG |
| | | K |
| 28 | IgG4 dK + S228P + | ASTKGPSVFPLAPCSRSTSESTAALGCLVKDYFPEPVTVSW |
| | 252Y/254T/256E HC | NSGALTSGVHTFPAVLQSSGLYSLSSVVTVPSSSLGTKTYTC |
| | constant region | NVDHKPSNTKVDKRVESKYGPPCPPCPAPEFLGGPSVFLFP |
| | | PKPKDTLYITREPEVTCVVVDVSQEDPEVQFNWYVDGVEV |
| | | HNAKTKPREEQFNSTYRVVSVLTVLHQDWLNGKEYKCKVS |
| | | NKGLPSSIEKTISKAKGQPREPQVYTLPPSQEEMTKNQVSL |
| | | TCLVKGFYPSDIAVEWESNGQPENNYKTTPPVLDSDGSFFL |
| | | YSRLTVDKSRWQEGNVFSCSVMHEALHNHYTQKSLSLSLG |
| 29 | Lambda LC constant | QPKAAPSVTLFPPSSEELQANKATLVCLISDFYPGAVTVAW |
| | region | KADSSPVKAGVETTTPSKQSNNKYAASSYLSLTPEQWKSH |
| | | RSYSCQVTHEGSTVEKTVAPTECS |
| 30 | 320-587 Heavy | caggtgcagctggtgcagtccggcgccgaggtgaagaaacccggcgcct |
| | Chain Variable | ccgtgaaggtgtcctgcaaggccagcggctacaccttcacctcctacgac |
| | Region | atcaactgggtgaggcaggcccccggccagggcctggagtggatgggct |
| | | ggctgaaccccaactccggctacaccggctacgcccagaagttccagggc |
| | | agggtgaccatgaccgccgacaggtccacctccaccgcctacatggagct |
| | | gtccagcctgaggtccgaggacaccgccgtgtactattgcgccagggagg |
| | | tgcccgagaccgctgccttcgagtactggggccagggcaccctggtgacc |
| | | gtgtccagc |
| 31 | 320-587 Heavy | caggtgcagctggtgcagtccggcgccgaggtgaagaaacccggcgcct |
| | Chain | ccgtgaaggtgtcctgcaaggccagcggctacaccttcacctcctacgac |
| | | atcaactgggtgaggcaggcccccggccagggcctggagtggatgggct |
| | | ggctgaaccccaactccggctacaccggctacgcccagaagttccagggc |
| | | agggtgaccatgaccgccgacaggtccacctccaccgcctacatggagct |
| | | gtccagcctgaggtccgaggacaccgccgtgtactattgcgccagggagg |
| | | tgcccgagaccgctgccttcgagtactggggccagggcaccctggtgacc |
| | | gtgtccagcgcctccaccaagggccccagcgtgttccccctggcccccag |
| | | ctccaagtccaccagcggcggaaccgccgctctgggctgcctggtgaagg |
| | | actacttccccgagcccgtgaccgtgagctggaactccggcgccctgacct |
| | | ccggcgtgcacaccttccccgccgtgctgcagtccagcggcctgtactccc |
| | | tgagctccgtggtcaccgtgccctcctctagcctgggcacccagacctaca |
| | | tctgcaacgtgaaccacaagccctccaacaccaaggtggacaaaaaggt |
| | | ggagcccaagtcctgcgacaagactcacacctgtcctccctgccccgccc |
| | | ccgagctgctcggcggaccctccgtgttcctgttcccacccaagcccaagg |
| | | acaccctgatgatctccaggacccccgaggtgacctgcgtggtcgtggac |
| | | gtgtcccacgaggaccctgaggtgaagttcaactggtacgtggacggcgt |
| | | ggaggtgcacaacgccaagaccaagcccagggaggaacagtacaactc |
| | | cacctaccgggtcgtgtccgtgctgaccgtcctgcaccaggactggctgaa |
| | | cggcaaggagtacaagtgcaaggtgtccaacaaggccctgcccgccccc |
| | | atcgagaagaccatctccaaggccaagggccagcctcgggagccccagg |
| | | tgtacacactgcccccttccagggacgagctgaccaagaaccaggtgtcc |
| | | ctgacctgtctggtgaagggcttctacccctccgacatcgccgtggagtgg |
| | | gagtccaacggccagcccgagaacaattacaagaccacacctcccgtcct |
| | | ggactccgacggctccttctttctgtactccaagctgaccgtggacaagtc |
| | | caggtggcagcaaggcaacgtgttctcctgctccgtgatgcacgaggccc |
| | | tgcacaaccactacacccagaagtccctgagcctgtcccccggc |
| 32 | 320-587 Light Chain | cagagcgtgctgacacagcctccatccgtgtctggcgcccctggccagag |
| | Variable Region | agtgaccatcagctgcaccagcagcagcagcgacatcggagccggcctg |
| | | ggcgtgcactggtatcagcagctgcctggcaccgcccccaagctgctgat |
| | | cgagggctactacaaccggcccagcggcgtgcccgaccggtttagcggc |
| | | agcaagagcggcaccagcgccagcctgacaatcaccggcctgctgcccg |
| | | aggacgagggcgactactactgccagagctgggacggcaccctgagcgc |
| | | cctgttcggcggaggcaccaagctgaccgtcctaggt |
| 33 | 320-587 Light Chain | cagagcgtgctgacacagcctccatccgtgtctggcgcccctggccagag |
| | | agtgaccatcagctgcaccagcagcagcagcgacatcggagccggcctg |
| | | ggcgtgcactggtatcagcagctgcctggcaccgcccccaagctgctgat |
| | | cgagggctactacaaccggcccagcggcgtgcccgaccggtttagcggc |
| | | agcaagagcggcaccagcgccagcctgacaatcaccggcctgctgcccg |
| | | aggacgagggcgactactactgccagagctgggacggcaccctgagcgc |
| | | cctgttcggcggaggcaccaagctgaccgtcctaggtcagcccaaggccg |
| | | ctcccagcgtgaccctgttccccccaagcagcgaggaactgcaggccaac |
| | | aaggccaccctggtgtgcctgatcagcgacttctaccctggggccgtgacc |
| | | gtggcctggaaggccgatagcagccctgtgaaggccggcgtggaaacca |
| | | ccaccccctccaagcagagcaacaacaaatacgccgccagcagctacct |
| | | gtccctgacccccgagcagtggaagtcccaccggtcctacagctgccagg |
| | | tgacacacgagggcagcaccgtggaaaagaccgtggcccccaccgagtg |
| | | cagc |
| 34 | human TL1A | LKGQEFAPSHQQVYAPLRADGDKPRAHLTVVRQTPTQHF |
| | | KNQFPALHWEHELGLAFTKNRMNYTNKFLLIPESGDYFIYS |
| | | QVTFRGMTSECSEIRQAGRPNKPDSITVVITKVTDSYPEPT |
| | | QLLMGTKSVCEVGSNWFQPIYLGAMFSLQEGDKLMVNV |
| | | SDISLVDYTKEDKTFFGAFLL |
| 35 | human TL1A | LKGQEFAPSHQQVYAPLRADGDKPRAHLTVVRQTPTQHF |
| | | KNQFPALHWEHELGLAFTKNRMNYTNKFLLIPESGDYFIYS |
| | | QVTFAGMTSECSEIRQAGRPNKPDSITVVITKVTDSYPEPT |
| | | QLLMGTKSVCEVGSNWFQPIYLGAMFSLQEGDKLMVNV |
| | | SDISLVDYTKEDKTFFGAFLL |
| 36 | human TL1A | DYKDDDDKGSHHHHHHHHGSGSLVPRGSGSLKGQEFAPS |
| | | HQQVYAPLRADGDKPRAHLTVVRQTPTQHFKNQFPALH |
| | | WEHELGLAFTKNRMNYTNKFLLIPESGDYFIYSQVTFRGM |
| | | TSECSEIRQAGRPNKPDSITVVITKVTDSYPEPTQLLMGTKS |
| | | VCEVGSNWFQPIYLGAMFSLQEGDKLMVNVSDISLVDYTK |
| | | EDKTFFGAFLL |
| 37 | cynomolgus monkey | LKGQEFAPSHQQVYAPLRADGDKPRAHLTVVRQTPTQHL |
| | TL1A | KNQFPALHWEHELGLAFTKNRMNYTNKFLLIPESGDYFVY |
| | | SQVTFRGMTSECSEIRQAGRPNKPDSITVVITKVTDSYPEPT |
| | | QLLMGTKSVCEVGSNWFQPIYLGAMFSLQEGDKLMVNV |
| | | SDISLVDYTKEDKTFFGAFLL |
| 38 | mouse TL1A | LRAITEERSEPSPQQVYSPPRGKPRAHLTIKKQTPAPHLKN |
| | | QLSALHWEHDLGMAFTKNGMKYINKSLVIPESGDYFIYSQI |
| | | TFRGTTSVCGDISRGRRPNKPDSITMVITKVADSYPEPARLL |
| | | TGSKSVCEISNNWFQSLYLGATFSLEEGDRLMVNVSDISLV |
| | | DYTKEDKTFFGAFLL |
| 39 | rat TL1A | FPTVTEERSAPSAQPVYTPSRDKPKAHLTIMRQTPVPHLKN |
| | | ELAALHWENNLGMAFTKNRMNYTNKFLVIPESGDYFIYSQ |
| | | ITFRGTTSECGDISRVRRPKKPDSITVVITKVADSYPEPAHLL |
| | | TGTKSVCEISSNWFQPIYLGAMFSLEEGDRLMVNVSDISLV |
| | | DYTKEDKTFFGAFLI |
| 40 | guinea pig TL1A | INEQRFGPSYQRVYTPLRDDRDKPRAHLTVVRQTPTQHLK |
| | | NQFPALHWEHELGLAFTKNRMNYTNKFLVIPETGDYFVYS |
| | | QITFRGTTSECGISPGRQQNKPDSIFVVITKVTDSYPEPSQLL |
| | | TGTKSVCEISSNWFQPLYLGAMFSLQEGDKLMVNVSDISL |
| | | VDYTKEDKTFFGAFLL |
| 41 | cat TL1A | PKGREFGPSHQRAYTSPGAGGDKPRAHLTVVRQTPTQPLK |
| | | NQFPALHWEHELGLAFIKNRMNYTNKFLVIPESGDYFVYS |
| | | QVTFRGTTSECGEIRQGSRLNKPDSIIVVITKVTDSYPEPTQL |
| | | LMGTKSVCEVGSNWFQPIYLGAMFSLQEGDKLMVNVSDI |
| | | SLVDYTKEDKTFFGAFLL |
| 42 | pig TL1A | PKGQELGPSHQRVYAPPGAGRDKPRAHLTVVRQTSTEPLK |
| | | NQFPALHWEHELGLAFTKNRMNYTNKFLVIPESGDYFIYS |
| | | QVTFRGTTSECGEISQERRLNKPDSIIVVITKVTDSYPEPTQL |
| | | LMGTKSVCEIGSNWFQPIYLGAMFSLHEGDKLMVNVSDIS |
| | | LVDYTKEDKTFFGAFLL |
| 43 | rabbit TL1A | LKGREFGPSQQRAYMPLRADGNKPRAHLTAVKQTPTQPL |
| | | RNHFPALHWEHELGLAFTKNRMNYTNKFLVIPESGDYFVY |
| | | SQVTFRGTTSECGVINQRRRQTKPDSIVVVITKVTDNYPEP |
| | | AQLLTGTKSVCEMGNWFQPIYLGAMFSLEEGDKLMVNVS |
| | | DVSLVDYTKEDKTFFGAFLL |
| 44 | dog TL1A | PKGQEFGHSHQRAYASPRAGGDKPRAHLTVVRQSPTQPL |
| | | ESLFPALHWEHELGLAFTKNRMNYTNKFLVIPESGDYFVYS |
| | | QVTFRGTTSECGEARQGSRLNKPDSIIVVITKVTDSYPEPTQ |
| | | LLMGTKSVCEVGSNWFQPIYLGAMFSLQEGDKLMVNVSD |
| | | ISLVDYTKEDKTFFGAFLL |

1. A pharmaceutical formulation, comprising:
(a) about 100 mg/mL to about 250 mg/mL of an antibody or antigen-binding fragment thereof that specifically binds to TNF-like ligand 1A (TL1A); wherein the antibody or antigen-binding fragment thereof comprises:
a heavy chain variable region CDR1 comprising the amino acid sequence of SEQ ID NO: 1, a heavy chain variable region CDR2 comprising the amino acid sequence of SEQ ID NO: 2, a heavy chain variable region CDR3 comprising the amino acid sequence of SEQ ID NO: 3, a light chain variable region CDR1 comprising the amino acid sequence of SEQ ID NO: 4, a light chain variable region CDR2 comprising the amino acid sequence of SEQ ID NO: 5, and a light chain variable region CDR3 comprising the amino acid sequence of SEQ ID NO: 6;
(b) about 5 mM to about 15 mM Histidine;
(c) about 50 mM to about 150 mM Arginine-Hydrochloride (Arg-HCl); and
(d) about 2.5% (w/v) to about 7.5% (w/v) Sucrose.

2. The pharmaceutical formulation of claim 1, wherein the antibody or antigen-binding fragment thereof comprises a heavy chain variable region comprising the amino acid sequence of SEQ ID NO: 7 and a light chain variable region comprising the amino acid sequence of SEQ ID NO: 8.

3. The pharmaceutical formulation of claim 1, wherein the antibody or antigen-binding fragment comprises an IgG1 constant region.

4. The pharmaceutical formulation of claim 1, wherein the antibody or antigen-binding fragment thereof comprises a heavy chain comprising the amino acid sequence of SEQ ID NO: 9 and a light chain comprising the amino acid sequence of SEQ ID NO: 10.

5. The pharmaceutical formulation of claim 1 comprising about 100 mg/mL, about 150 mg/mL, about 200 mg/mL, about 225 mg/mL, or about 250 mg/mL of the antibody or antigen-binding fragment thereof.

6. (canceled)

7. The pharmaceutical formulation of claim 1, comprising about 200 mg/mL of the antibody or antigen-binding fragment thereof.

8. (canceled)

9. (canceled)

10. The pharmaceutical formulation of claim 1, comprising about 5 mM, about 10 mM, or about 15 mM Histidine.

11. The pharmaceutical formulation of claim 1, comprising about 10 mM Histidine.

12. (canceled)

13. The pharmaceutical formulation of claim 1, comprising about 50 mM, about 100 mM, or about 150 mM Arginine-Hydrochloride (Arg-HCl).

14. The pharmaceutical formulation of claim 1, comprising about 100 mM Arginine-Hydrochloride (Arg-HCl).

15. (canceled)

16. The pharmaceutical formulation of claim 1, comprising about 2.5% (w/v), about 5% (w/v), or about 7.5% (w/v) Sucrose.

17. The pharmaceutical formulation of claim 1, comprising about 2.5% (w/v) Sucrose.

18. (canceled)

19. (canceled)

20. (canceled)

21. (canceled)

22. The pharmaceutical formulation of claim 1, comprising about 200 mg/mL of the antibody or antigen binding fragment thereof, about 10 mM Histidine, about 100 mM Arginine-Hydrochloride (Arg-HCl), and about 2.5% (w/v) Sucrose.

23. (canceled)

24. (canceled)

25. The pharmaceutical formulation of claim 1, wherein the pharmaceutical formulation is lyophilized or is liquid.

26. (canceled)

27. The pharmaceutical formulation of claim 1, having:
(a) a pH of 6.0±0.5 after storage at room temperature for 24 hours or at 2-8° C. for 24 hours, 72 hours, or 10 days,
(b) an osmolality of from 200 mOsm/kg to 500 mOsm/kg after storage at room temperature for 24 hours or at 2-8° C. for 24 hours, 72 hours, or 10 days;
(c) at least 99% antibody monomer content after storage at 2-8° C. for 24 hours, 72 hours, or 10 days;
(d) no significant change in charge heterogeneity profile after storage at 2-8° C. for 24 hours, 72 hours, or 10 days;
(e) no significant change in purity after storage at room temperature for 24 hours or at 2-8° C. for 24 hours, 72 hours, or 10 days;
(f) at least 90% purity after storage at room temperature for 24 hours or at 2-8° C. for 24 hours, 72 hours, or 10 days;
(g) no significant change in particle concentration after storage at room temperature for 24 hours or at 2-8° C. for 24 hours, 72 hours, or 10 days;
(h) no significant difference in visual appearance after storage at 2-8° C. for up to 36 months;
(i) no significant difference in protein concentration, osmolality, or viscosity after storage at 2-8° C., 25° C. or 40° C. for up to 36 months;
(j) ≥95% monomer content, ≤5.0% dimer content, or no significant difference in low molecular weight species content after storage at 2-8° C. for up to 36 months;
(k) ≥90% purity after storage at 2-8° C. for up to 36 months;
(l) from 50% to 90% main species content, from 10% to 40% acidic species content, and/or from 0% to 20% basic species content after storage at 2-8° C. for up to 36 months;
(m) no significant difference in sub-visible particle content after storage at 2-8° C., 25° C., or 40° C. for up to 36 months;
(n) from 70% to 135% relative potency measured by enzyme-linked immunosorbent assay (ELISA) after storage at 2-8° C. for up to 36 months;
(o) no significant difference in thermal stability after storage at 2-8° C., 25° C., or 40° C. for up to 6 months;
(p) no significant difference in thermal stability after storage at 2-8° C. for up to 36 months;
(q) no significant difference in secondary and/or tertiary protein structure after storage at 2-8° C., 25° C., or 40° C. for up to 3 months; and/or
(r) no significant difference in secondary protein structure after storage at 2-8° C. for up to 36 months.

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. The pharmaceutical formulation of claim 4, having no significant difference in oxidation of methionine 81 and/or methionine 254 of SEQ ID NO: 9, and/or deamidation of asparagine 317 of SEQ ID NO: 9 after storage at 2-8° C., 25° C., or 40° C. for up to 36 months.

41. (canceled)

42. (canceled)

43. (canceled)

44. (canceled)

45. (canceled)

46. (canceled)

47. The pharmaceutical formulation of claim 1, wherein the antibody or antigen-binding fragment thereof was produced in a Chinese hamster ovary cell.

48. A container comprising the pharmaceutical formulation of claim 1.

49. The container of claim 48, wherein the container is a glass vial or a syringe.

50. (canceled)

51. (canceled)

52. A method of treating a disease in a subject in need thereof, the method comprising administering to the subject the pharmaceutical formulation of claim 1, optionally wherein the disease is a respiratory tract disease, a gastrointestinal disease, a skin disease, or an arthritis.

53. (canceled)

54. (canceled)

55. (canceled)

56. (canceled)

57. (canceled)

58. (canceled)

59. (canceled)


SEQUENCE LISTING (44 sequences)

SEQ ID NO: 1 - protein, 10 aa, synthetic construct
  REGION 1..10: note = 320-587 Heavy Chain CDR1
GYTFTSYDIN

SEQ ID NO: 2 - protein, 10 aa, synthetic construct
  REGION 1..10: note = 320-587 Heavy Chain CDR2
WLNPNSGYTG

SEQ ID NO: 3 - protein, 10 aa, synthetic construct
  REGION 1..10: note = 320-587 Heavy Chain CDR3
EVPETAAFEY

SEQ ID NO: 4 - protein, 14 aa, synthetic construct
  REGION 1..14: note = 320-587 Light Chain CDR1
TSSSSDIGAGLGVH

SEQ ID NO: 5 - protein, 7 aa, synthetic construct
  REGION 1..7: note = 320-587 Light Chain CDR2
GYYNRPS

SEQ ID NO: 6 - protein, 10 aa, synthetic construct
  REGION 1..10: note = 320-587 Light Chain CDR3
QSWDGTLSAL

SEQ ID NO: 7 - protein, 119 aa, synthetic construct
  REGION 1..119: note = 320-587 Heavy Chain Variable Region
QVQLVQSGAEVKKPGASVKVSCKASGYTFTSYDINWVRQAPGQGLEWMGWLNPNSGYTGYAQKFQGRVTMTADRSTSTAYMELSSLRSEDTAVYYCAREVPETAAFEYWGQGTLVTVSS

SEQ ID NO: 8 - protein, 111 aa, synthetic construct
  REGION 1..111: note = 320-587 Light Chain Variable Region
QSVLTQPPSVSGAPGQRVTISCTSSSSDIGAGLGVHWYQQLPGTAPKLLIEGYYNRPSGVPDRFSGSKSGTSASLTITGLLPEDEGDYYCQSWDGTLSALFGGGTKLTVLG

SEQ ID NO: 9 - protein, 448 aa, synthetic construct
  REGION 1..448: note = 320-587 Heavy Chain
QVQLVQSGAEVKKPGASVKVSCKASGYTFTSYDINWVRQAPGQGLEWMGWLNPNSGYTGYAQKFQGRVTMTADRSTSTAYMELSSLRSEDTAVYYCAREVPETAAFEYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 10 - protein, 216 aa, synthetic construct
  REGION 1..216: note = 320-587 Light Chain
QSVLTQPPSVSGAPGQRVTISCTSSSSDIGAGLGVHWYQQLPGTAPKLLIEGYYNRPSGVPDRFSGSKSGTSASLTITGLLPEDEGDYYCQSWDGTLSALFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 11 - protein, 119 aa, synthetic construct
  REGION 1..119: note = 320-179 Heavy Chain Variable Region
QVQLVQSGAEVKKPGASVKVSCKASGYTFTSYDINWVRQAPGQGLEWMGWLNPNSGNTGYAQKFQGRVTMTADRSTSTAYMELSSLRSEDTAVYYCAREVPETAAFEYWGQGTLVTVSS

SEQ ID NO: 12 - protein, 111 aa, synthetic construct
  REGION 1..111: note = 320-179 Light Chain Variable Region
QSVLTQPPSVSGAPGQRVTISCTSSSSDIGAGLGVHWYQQLPGTAPKLLIEGYYNRPSGVPDRFSGSKSGTSASLTITGLLPEDEGDYYCQSYDGTLSALFGGGTKLTVLG

SEQ ID NO: 13 - protein, 330 aa, synthetic construct [sequence omitted]

SEQ ID NO: 14 - protein, 329 aa, synthetic construct
  REGION 1..329: note = IgG1 dK HC constant region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 15 - protein, 330 aa, synthetic construct
  REGION 1..330: note = IgG1 252Y/254T/256E HC constant region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 329 aa, synthetic construct
  REGION 1..329: note = IgG1 dK + 252Y/254T/256E HC constant region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 17 - protein, 330 aa, synthetic construct
  REGION 1..330: note = IgG1 L234A, L235A, G237A HC constant region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - protein, 329 aa, synthetic construct
  REGION 1..329: note = IgG1 dK + L234A, L235A, G237A HC constant region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 19 - protein, 330 aa, synthetic construct
  REGION 1..330: note = IgG1 L235A/G237A HC constant region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - protein, 329 aa, synthetic construct
  REGION 1..329: note = IgG1 dK + L235A/G237A HC constant region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 21 - protein, 326 aa, synthetic construct
  REGION 1..326: note = IgG2 HC constant region
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 22 - protein, 325 aa, synthetic construct
  REGION 1..325: note = IgG2 dK HC constant region
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 23 - protein, 326 aa, synthetic construct
  REGION 1..326: note = IgG2 A330S/P331S HC constant region
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPSSIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 24 - protein, 325 aa, synthetic construct
  REGION 1..325: note = IgG2 dK + A330S/P331S HC constant region
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPSSIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 25 - protein, 327 aa, synthetic construct
  REGION 1..327: note = IgG4 HC constant region
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 26 - protein, 326 aa, synthetic construct
  REGION 1..326: note = IgG4 dK + S228P HC constant region
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 27 - protein, 327 aa, synthetic construct
  REGION 1..327: note = IgG4 S228P + 252Y/254T/256E HC constant region
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 28 - protein, 326 aa, synthetic construct
  REGION 1..326: note = IgG4 dK + S228P + 252Y/254T/256E HC constant region
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 29 - protein, 105 aa, synthetic construct
  REGION 1..105: note = Lambda LC constant region
QPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 30 - DNA, 357 bp, synthetic construct
  source 1..357: mol_type = other DNA
  misc_feature 1..357: note = 320-587 Heavy Chain Variable Region
caggtgcagctggtgcagtccggcgccgaggtgaagaaacccggcgcctccgtgaaggtgtcctgcaaggccagcggctacaccttcacctcctacgacatcaactgggtgaggcaggcccccggccagggcctggagtggatgggctggctgaaccccaactccggctacaccggctacgcccagaagttccagggcagggtgaccatgaccgccgacaggtccacctccaccgcctacatggagctgtccagcctgaggtccgaggacaccgccgtgtactattgcgccagggaggtgcccgagaccgctgccttcgagtactggggccagggcaccctggtgaccgtgtccagc

SEQ ID NO: 31 - DNA, 1344 bp, synthetic construct
  source 1..1344: mol_type = other DNA
  misc_feature 1..1344: note = 320-587 Heavy Chain
caggtgcagctggtgcagtccggcgccgaggtgaagaaacccggcgcctccgtgaaggtgtcctgcaaggccagcggctacaccttcacctcctacgacatcaactgggtgaggcaggcccccggccagggcctggagtggatgggctggctgaaccccaactccggctacaccggctacgcccagaagttccagggcagggtgaccatgaccgccgacaggtccacctccaccgcctacatggagctgtccagcctgaggtccgaggacaccgccgtgtactattgcgccagggaggtgcccgagaccgctgccttcgagtactggggccagggcaccctggtgaccgtgtccagcgcctccaccaagggccccagcgtgttccccctggcccccagctccaagtccaccagcggcggaaccgccgctctgggctgcctggtgaaggactacttccccgagcccgtgaccgtgagctggaactccggcgccctgacctccggcgtgcacaccttccccgccgtgctgcagtccagcggcctgtactccctgagctccgtggtcaccgtgccctcctctagcctgggcacccagacctacatctgcaacgtgaaccacaagccctccaacaccaaggtggacaaaaaggtggagcccaagtcctgcgacaagactcacacctgtcctccctgccccgcccccgagctgctcggcggaccctccgtgttcctgttcccacccaagcccaaggacaccctgatgatctccaggacccccgaggtgacctgcgtggtcgtggacgtgtcccacgaggaccctgaggtgaagttcaactggtacgtggacggcgtggaggtgcacaacgccaagaccaagcccagggaggaacagtacaactccacctaccgggtcgtgtccgtgctgaccgtcctgcaccaggactggctgaacggcaaggagtacaagtgcaaggtgtccaacaaggccctgcccgcccccatcgagaagaccatctccaaggccaagggccagcctcgggagccccaggtgtacacactgcccccttccagggacgagctgaccaagaaccaggtgtccctgacctgtctggtgaagggcttctacccctccgacatcgccgtggagtgggagtccaacggccagcccgagaacaattacaagaccacacctcccgtcctggactccgacggctccttctttctgtactccaagctgaccgtggacaagtccaggtggcagcaaggcaacgtgttctcctgctccgtgatgcacgaggccctgcacaaccactacacccagaagtccctgagcctgtcccccggc

SEQ ID NO: 32 - DNA, 333 bp, synthetic construct
  source 1..333: mol_type = other DNA
  misc_feature 1..333: note = 320-587 Light Chain Variable Region
cagagcgtgctgacacagcctccatccgtgtctggcgcccctggccagagagtgaccatcagctgcaccagcagcagcagcgacatcggagccggcctgggcgtgcactggtatcagcagctgcctggcaccgcccccaagctgctgatcgagggctactacaaccggcccagcggcgtgcccgaccggtttagcggcagcaagagcggcaccagcgccagcctgacaatcaccggcctgctgcccgaggacgagggcgactactactgccagagctgggacggcaccctgagcgccctgttcggcggaggcaccaagctgaccgtcctaggt

SEQ ID NO: 33 - DNA, 648 bp, synthetic construct
  source 1..648: mol_type = other DNA
  misc_feature 1..648: note = 320-587 Light Chain
cagagcgtgctgacacagcctccatccgtgtctggcgcccctggccagagagtgaccatcagctgcaccagcagcagcagcgacatcggagccggcctgggcgtgcactggtatcagcagctgcctggcaccgcccccaagctgctgatcgagggctactacaaccggcccagcggcgtgcccgaccggtttagcggcagcaagagcggcaccagcgccagcctgacaatcaccggcctgctgcccgaggacgagggcgactactactgccagagctgggacggcaccctgagcgccctgttcggcggaggcaccaagctgaccgtcctaggtcagcccaaggccgctcccagcgtgaccctgttccccccaagcagcgaggaactgcaggccaacaaggccaccctggtgtgcctgatcagcgacttctaccctggggccgtgaccgtggcctggaaggccgatagcagccctgtgaaggccggcgtggaaaccaccaccccctccaagcagagcaacaacaaatacgccgccagcagctacctgtccctgacccccgagcagtggaagtcccaccggtcctacagctgccaggtgacacacgagggcagcaccgtggaaaagaccgtggcccccaccgagtgcagc

SEQ ID NO: 34 - protein, 180 aa, synthetic construct
  REGION 1..180: note = human TL1A
LKGQEFAPSHQQVYAPLRADGDKPRAHLTVVRQTPTQHFKNQFPALHWEHELGLAFTKNRMNYTNKFLLIPESGDYFIYSQVTFRGMTSECSEIRQAGRPNKPDSITVVITKVTDSYPEPTQLLMGTKSVCEVGSNWFQPIYLGAMFSLQEGDKLMVNVSDISLVDYTKEDKTFFGAFLL

SEQ ID NO: 35 - protein, 180 aa, synthetic construct
  REGION 1..180: note = human TL1A
LKGQEFAPSHQQVYAPLRADGDKPRAHLTVVRQTPTQHFKNQFPALHWEHELGLAFTKNRMNYTNKFLLIPESGDYFIYSQVTFAGMTSECSEIRQAGRPNKPDSITVVITKVTDSYPEPTQLLMGTKSVCEVGSNWFQPIYLGAMFSLQEGDKLMVNVSDISLVDYTKEDKTFFGAFLL

SEQ ID NO: 36 - protein, 210 aa, synthetic construct
  REGION 1..210: note = human TL1A
DYKDDDDKGSHHHHHHHHGSGSLVPRGSGSLKGQEFAPSHQQVYAPLRADGDKPRAHLTVVRQTPTQHFKNQFPALHWEHELGLAFTKNRMNYTNKFLLIPESGDYFIYSQVTFRGMTSECSEIRQAGRPNKPDSITVVITKVTDSYPEPTQLLMGTKSVCEVGSNWFQPIYLGAMFSLQEGDKLMVNVSDISLVDYTKEDKTFFGAFLL

SEQ ID NO: 37 - protein, 180 aa, synthetic construct
  REGION 1..180: note = cynomolgus monkey TL1A
LKGQEFAPSHQQVYAPLRADGDKPRAHLTVVRQTPTQHLKNQFPALHWEHELGLAFTKNRMNYTNKFLLIPESGDYFVYSQVTFRGMTSECSEIRQAGRPNKPDSITVVITKVTDSYPEPTQLLMGTKSVCEVGSNWFQPIYLGAMFSLQEGDKLMVNVSDISLVDYTKEDKTFFGAFLL

SEQ ID NO: 38 - protein, 180 aa, synthetic construct
  REGION 1..180: note = mouse TL1A
LRAITEERSEPSPQQVYSPPRGKPRAHLTIKKQTPAPHLKNQLSALHWEHDLGMAFTKNGMKYINKSLVIPESGDYFIYSQITFRGTTSVCGDISRGRRPNKPDSITMVITKVADSYPEPARLLTGSKSVCEISNNWFQSLYLGATFSLEEGDRLMVNVSDISLVDYTKEDKTFFGAFLL

SEQ ID NO: 39 - protein, 180 aa, synthetic construct
  REGION 1..180: note = rat TL1A
FPTVTEERSAPSAQPVYTPSRDKPKAHLTIMRQTPVPHLKNELAALHWENNLGMAFTKNRMNYTNKFLVIPESGDYFIYSQITFRGTTSECGDISRVRRPKKPDSITVVITKVADSYPEPAHLLTGTKSVCEISSNWFQPIYLGAMFSLEEGDRLMVNVSDISLVDYTKEDKTFFGAFLI

SEQ ID NO: 40 - protein, 179 aa, synthetic construct
  REGION 1..179: note = guinea pig TL1A
INEQRFGPSYQRVYTPLRDDRDKPRAHLTVVRQTPTQHLKNQFPALHWEHELGLAFTKNRMNYTNKFLVIPETGDYFVYSQITFRGTTSECGISPGRQQNKPDSIFVVITKVTDSYPEPSQLLTGTKSVCEISSNWFQPLYLGAMFSLQEGDKLMVNVSDISLVDYTKEDKTFFGAFLL

SEQ ID NO: 41 - protein, 180 aa, synthetic construct
  REGION 1..180: note = cat TL1A
PKGREFGPSHQRAYTSPGAGGDKPRAHLTVVRQTPTQPLKNQFPALHWEHELGLAFIKNRMNYTNKFLVIPESGDYFVYSQVTFRGTTSECGEIRQGSRLNKPDSIIVVITKVTDSYPEPTQLLMGTKSVCEVGSNWFQPIYLGAMFSLQEGDKLMVNVSDISLVDYTKEDKTFFGAFLL

SEQ ID NO: 42 - protein, 180 aa, synthetic construct
  REGION 1..180: note = pig TL1A
PKGQELGPSHQRVYAPPGAGRDKPRAHLTVVRQTSTEPLKNQFPALHWEHELGLAFTKNRMNYTNKFLVIPESGDYFIYSQVTFRGTTSECGEISQERRLNKPDSIIVVITKVTDSYPEPTQLLMGTKSVCEIGSNWFQPIYLGAMFSLHEGDKLMVNVSDISLVDYTKEDKTFFGAFLL

SEQ ID NO: 43 - protein, 179 aa, synthetic construct
  REGION 1..179: note = rabbit TL1A
LKGREFGPSQQRAYMPLRADGNKPRAHLTAVKQTPTQPLRNHFPALHWEHELGLAFTKNRMNYTNKFLVIPESGDYFVYSQVTFRGTTSECGVINQRRRQTKPDSIVVVITKVTDNYPEPAQLLTGTKSVCEMGNWFQPIYLGAMFSLEEGDKLMVNVSDVSLVDYTKEDKTFFGAFLL

SEQ ID NO: 44 - protein, 180 aa, synthetic construct
  REGION 1..180: note = dog TL1A
PKGQEFGHSHQRAYASPRAGGDKPRAHLTVVRQSPTQPLESLFPALHWEHELGLAFTKNRMNYTNKFLVIPESGDYFVYSQVTFRGTTSECGEARQGSRLNKPDSIIVVITKVTDSYPEPTQLLMGTKSVCEVGSNWFQPIYLGAMFSLQEGDKLMVNVSDISLVDYTKEDKTFFGAFLL